(19) United States
(12) Patent Application Publication
(10) Pub. No.: US 2017/0283860 A1
(43) Pub. Date: Oct. 5, 2017

(54) METHODS AND COMPOSITIONS FOR THE REMOVAL OF ALDEHYDE ADDUCTS AND CROSSLINKS FROM BIOMOLECULES

(71) Applicant: The Board of Trustees of the Leland Stanford Junio University, Stanford, CA (US)

(72) Inventors: Eric Todd Kool, Stanford, CA (US); Emily Harcourt, Hinesburg, VT (US); Saswata Karmaker, Somerville, MA (US)

(21) Appl. No.: 15/511,407

(22) PCT Filed: Sep. 15, 2015
(86) PCT No.: PCT/US15/50254

§ 371 Date: Mar. 15, 2017

Related U.S. Application Data
(60) Provisional application No. 62/051,211, filed on Sep. 16, 2014.
(60) Provisional application No. 62/168,625, filed on May 29, 2015.

(51) Int. Cl. C12Q 1/68

(52) U.S. Cl. CPC C12Q 1/6806

(57) ABSTRACT

Methods are provided for reducing the number of aldehyde adducts and/or crosslinks from fixed biomolecules. In some cases, subject methods include contacting a sample having aldehyde fixed biomolecules (e.g., a biological sample such as a formalin fixed paraffin embedded (FFPE) tissue sample) with an adduct reversal agent in an amount and for a period of time sufficient to reduce the number of aldehyde fixation related adducts and/or crosslinks in the sample. In some cases, the adduct reversal agent is a compound that includes an aromatic ring and at least one of: an amine group and a proton-donating group. In some cases, the adduct reversal agent is a compound selected from the compounds of Table 1. Compositions and kits for practicing the subject methods are also provided.

### CROSS-REFERENCE

This application claims the benefit of U.S. Provisional Patent Application Nos. 62/051,211 filed Sep. 16, 2014, and 62/168,625, filed May 29, 2015, each of which applications is incorporated herein by reference in its entirety.

### GOVERNMENT RIGHTS

This invention was made with Government support under contract GM068122 awarded by the National Institutes of Health. The Government has certain rights in the invention

### BACKGROUND

There is a major trend in medicine toward molecular characterization of disease, using this information to guide treatment. A large number of clinical tissue specimens (biopsies, surgical specimens) are prepared prior to analysis by fixation with formalin (formaldehyde), in formalin fixed paraffin embedded (FFPE) tissue block format. Fixation results in extensive addition of adducts to, and molecular crosslinks between, biomolecules in the sample, which greatly reduces the amount of signal that can be obtained on later molecular analysis. This limits the length of PCR amplicons that can be analyzed, hinders or destroys the quantitation of RNA transcripts, and greatly lowers antigen signals in immunohistochemistry. Current methods for treating formalin-fixed tissue prior to analysis involve heating in buffers (usually Tris), which may not remove all adducts, and conditions are harsh enough (60-70° C., several hours' incubation) that the RNA/DNA/protein is permanently damaged in the process. This problem is universally recognized by pathologists and physicians who use these samples.

There is a need for methods to remove adducts and/or crosslinks (e.g., formaldehyde adducts and/or crosslinks) rapidly and under mild conditions. There are (to our knowledge) no previous examples in the literature of using catalyst molecules to reverse adducts and/or crosslinks (with the exception of simple buffers). In addition, there are no previous examples of using molecules with a combination of aryl amine groups and acids to catalyze such adduct and/or crosslink removal. Such methods would constitute a major breakthrough in pathology in general, and more specifically in diagnosis, prognosis, and treatment of diseases such as cancer.

### SUMMARY

Methods are provided for using catalysts (adduct reversal agents) to reduce the number of adducts and/or crosslinks on aldehyde fixed biomolecules (biomolecules previously contacted with an aldehyde fixation reagent). Aspects of the methods include contacting a sample having aldehyde fixed biomolecules (e.g., formaldehyde fixed biomolecules) with an adduct reversal agent in an amount and for a period of time sufficient for reducing the number of aldehyde fixation related adducts and/or crosslinks in the sample. In some cases, the aldehyde is formaldehyde. As such, in some cases, subject methods include contacting a sample having formaldehyde fixed biomolecules with an adduct reversal agent in an amount and for a period of time sufficient to reduce the number of formaldehyde fixation related adducts and/or crosslinks in the sample.

In some cases the aldehyde fixed biomolecules are part of a biological sample. For example, in some cases, the aldehyde fixed biomolecules can be present in a cellular sample (e.g., a tissue sample) that was contacted with an aldehyde crosslinking (fixation) reagent (e.g., formaldehyde, glutaraldehyde, etc.). In some cases, subject methods include contacting a fixed biological sample (e.g., a tissue sample, e.g., a biopsy, a blood sample, etc.) with a subject adduct reversal agent. In some cases, a fixed biological sample is a formalin fixed paraffin embedded (FFPE) biological sample. As such, in some cases, subject methods include contacting a formaldehyde fixed biological sample (e.g., an FFPE biological sample) with an adduct reversal agent in an amount and for a period of time sufficient to reduce the number of formaldehyde fixation-related adducts and/or crosslinks in the sample.

In some cases, the adduct reversal agent is a compound that includes an amine group and a proton-donating group. For example, the amine group and the proton-donating group can be substituted on a cyclic group, such as an aromatic ring. In some cases, the adduct reversal agent is a compound that includes an aromatic ring and at least one of: an amine group and a proton-donating group. In some cases, the adduct reversal agent is a compound that includes an aromatic ring, an amine group, and a proton-donating group. In some cases, the adduct reversal agent is a compound selected from the compounds of Table 1. In some cases, the adduct reversal agent is a compound selected from Compounds 1 to 4. In some cases, the adduct reversal agent is Compound 4.

In some cases, the sample having the fixed biomolecules is contacted with an adduct reversal agent at a temperature in a range of from 15° C. to 85° C. (e.g, 15° C. to 80° C., 15° C. to 70° C., 15° C. to 60° C., 15° C. to 50° C., 15° C. to 40° C., 20° C. to 40° C., room temperature, etc.), or is contacted with an adduct reversal agent at room temperature. In some cases, the sample having the fixed biomolecules is contacted with an adduct reversal agent for a period of time in a range of from 20 minutes to 24 hours. In some cases, the sample having the fixed biomolecules is contacted with an adduct reversal agent for a period of time in a range of from 20 minutes to 12 hours. In some cases, the sample having the fixed biomolecules is contacted with an adduct reversal agent for a period of time in a range of from 20 minutes to 6 hours. In some cases, the sample having the fixed biomolecules is contacted with an adduct reversal agent in a solution buffered to a pH in a range of from 6 to 8 (e.g., 6.5 to 7.5). In some cases, the sample having the fixed biomolecules is contacted with an adduct reversal agent in a solution buffered to a pH that is at or near the pKa of the adduct reversal agent.

In some cases, the methods include a step of contacting the sample having aldehyde fixed biomolecules with a protease (i.e., a proteolytic enzyme) (e.g., trypsin, chymotrypsin, proteinase K, papain, pepsin, pronase, endoproteinase Lys-C, endoproteinase glu-C, and the like). For example, in some cases, the methods include a step of contacting an aldehyde fixed biological sample, (e.g., a formaldehyde fixed biological sample, an FFPE biological sample, etc.) with a protease (e.g., trypsin, chymotrypsin, proteinase K, papain, pepsin, pronase, endoproteinase Lys-C, endoproteinase glu-C, and the like). For example, in some cases, the protease is proteinase K, pepsin, or pronase. In some cases, the step of contacting the sample having aldehyde fixed biomolecules with a protease is conducted in the presence of a chaotropic agent. For example, the chaotropic agent is a salt such as guanidinium chloride or guanidinium thiocyanate. In some cases, the methods include a step of removing an embedding medium (e.g., paraffin) from an aldehyde fixed biological sample prior to, simultaneously with, or after contacting the sample with a subject adduct reversal agent. In some cases, the methods include, after contacting a sample having aldehyde fixed biomolecules with an adduct reversal agent, a step of detecting a biomolecule (e.g., RNA, DNA, protein) in the contacted biological sample. In some cases, said detecting includes PCR, nucleic acid sequencing, in situ hybridization, and/or or an antibody-based protein detection method. In some cases, said detecting includes quantification of the biomolecule, for example measurement of the amount or the concentration of the biomolecule (e.g. RNA, DNA, or protein).

Compositions and kits for practicing the subject methods are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1C. Formaldehyde adducts and catalysts in this study. (FIG. 1A) Adducts on N6 of adenine (R=ribose or deoxyribose); formaldehyde residues are shown in red. Similar adducts are formed on exocyclic amines of cytosine and guanine. (FIG. 1B) Transimination catalyst structures studied. (FIG. 1C) Structures of formaldehyde adducts (hemiaminal) of two additional RNA/DNA bases (G Guanine shown at left; and Adenine shown at right, R can be a DNA or RNA ribose).

FIG. 2A-2B. Relative rates of formaldehyde adduct reversal. (FIG. 2A) Relative rates of reversal of the hemiaminal adduct of dAMP in Tris buffer alone (pH 7) or with 10 mM added catalysts shown. (FIG. 2B) Rates of reversal of the aminal crosslink of AMP in phosphate buffer alone (pH 4.5) or with 10 mM added catalysts. Data are from three replicates each (error bars are standard deviations).

FIG. 3A-3B. Relative yields of adduct reversal from nucleotides after 1 h with Compound 4and with analogs having functional groups omitted (structures shown). (FIG. 3A) Reversal of the hemiaminal adduct of dAMP (pH 7, 16 mM catalyst, 37° C.) (left to right, compounds 11, 1, and 4); (FIG. 3B) Reversal of the aminal dimer of AMP (pH 4.5, 16 mM catalyst, 37° C.) (left to right, compounds 11, 1, and 4).

FIG. 4A-4B. Assessing formaldehyde adducts on an RNA strand by mass spectrometry. (FIG. 4A) Sequence of the self-complementary 16 mer RNA, which was designed to promote adducts and crosslinks on unpaired bases. (FIG. 4B) MALDI mass spectrum of formaldehyde-treated RNA, showing extensive adducts after 24 h treatment (up to 14 per strand, see inset) and little or no unmodified RNA (5181 Da) remaining. Unmodified DNA (4294 Da) is spiked in for reference.

FIG. 5A-5C. Improvement in reversal of RNA formaldehyde adducts after low-temperature incubation in the presence of Compound 4 (“Compound 4”). RNA pretreated with 10% formaldehyde (see FIG. 4A-4B) was used as starting material. (FIG. 5A) MALDI mass spectrum of 16 mer RNA oligonucleotide after 18 h treatment with Compound 4 (8 mM, pH 7, 37° C.), showing major recovered RNA peak. (FIG. 5B) Time course of RNA recovery, comparing 60° C. heating in Tris buffer (*) to 37° C. incubations with buffer alone (*) and with 8 mM Compound 4 alone (*). Error bars show standard deviation from 5 experiments. (FIG. 5C) Time course of crosslink reversal in dimerized RNA oligonucleotide, following uncrosslinking to monomer RNA by denaturing PAGE. Shown is data for incubation at pH 4.5, 37° C. in 16 mM citrate buffer (*) in comparison to treatment at pH 4.5, 37° C. with 16 mM Compound 4 M. Error bars show standard deviation from 3 or 4 experiments.

FIG. 6. Enhancement in recovery of RNAs from formalin-fixed, paraffin-embedded cell specimens using Compound 4 (20 mM) as compared with different incubation and isolation conditions. Amplifiable RNA yield is plotted for eight amplicons, and quantity is determined with a standard curve. Commercial kit (Qiagen AllPrep® DNA/RNA FFPE kit) results are shown in lane 1 (“no cat”) which uses a spin column for isolation and an 80° C., 0.25 h incubation step. A common literature procedure is shown in lane 5 (“PCI”)²⁸. The means of three independent experiments are shown, error bars indicating the standard deviation of variation in the qRT-PCR yield. SC: spin column isolation. PCI: Masuda protocol of phenol-chloroform-isoamyl alcohol extraction followed by heating in buffer. A.U.: arbitrary units. Significance for pairwise comparisons shown was tested using a 1-tailed paired samples t-test. *: P<0.05; **: P<0.01. Fold enhancements are given in red.

FIG. 7. ESI-MS data for hemiaminal formaldehyde adduct of dAMP (first) and dimer aminal adduct of AMP (second). Note that the hemiaminal is unstable and decomposed partially to dAMP while awaiting mass spectrometry. See peak assignments, which reflect a mixture of dAMP and dAMP-formaldehyde hemiaminal (peaks marked with red arrows).

FIG. 8. ¹H-NMR spectrum of aminal formaldehyde dimer of AMP. Peaks at 3.2 and 1.3 ppm are for residual TEA-acetate buffer after HPLC purification.

FIG. 9. Representative HPLC traces showing reversal of monoadduct of dAMP over a period of 4 h in the presence of buffer or catalysts. Conditions: 5 mM catalyst added to pH 7.0 Tris (30 mM).

FIG. 10. Representative HPLC traces showing reversal of crosslinked dimer aminal of AMP over a period of hours. Conditions: 5 mM catalyst added to pH 7.0 Tris buffer (30 mM).

FIG. 11A-11B. (FIG. 11A) Screen of potential catalysts in reversal of formaldehyde monoadduct. Conditions: 23° C., Tris buffer pH 7 (30 mM), 5 mM potential catalyst added. Note that some of the highly acidic catalysts overwhelm the buffering power of Tris at this concentration. Refer to Table 1 for Compound numbers. (FIG. 11B) Screen of potential catalysts in reversal of formaldehyde crosslinked dimer. Conditions: 23° C., Tris buffer pH 7 (30 mM), 5 mM potential catalyst. Note that some of the highly acidic catalysts (compounds 11-14) overwhelm the buffering power of Tris at this concentration. At right is zoomed-in plot showing the remaining compounds that are not highly acidic. Refer to Table 1 for Compound numbers.

FIG. 12. Slow reversal of dAMP formaldehyde monoadduct with varied buffers, showing that pH and buffer concentration have little effect. Conditions: phosphate buffer, 30 or 120 mM, pH 4.8 or 7.0, 23° C.

FIG. 13. Slow reversal of dimer aminal in the presence of varied buffers, showing pH and buffer concentration effects. Conditions: Tris, phosphate, or citrate buffer, concentration shown, pH varied as indicated, 23° C.

FIG. 14. Effect of buffer vs. catalysts on reversal of hemiaminal adduct of dAMP at pH 7. Note increasing rate of reaction with increasing catalyst concentrations, consistent with presence of catalyst at transition state.

FIG. 15. Effect of buffer vs. catalysts on reversal of crosslinked aminal adduct of AMP. Note increasing rate of reaction with increasing Compound 4 concentration, but no similar effect with Compound 3.

FIG. 16. Extended time course showing reversal of formaldehyde monoadduct of dAMP in the presence of varied catalysts. Conditions with catalysts: catalyst [16 mM] in 30 mM Tris buffer, titrated to pH 7.0, 37° C.

FIG. 17. Extended time course showing reversal of formaldehyde dimer (aminal) of AMP in the presence of varied catalysts. Conditions: catalysts [16 mM] titrated to pH 4.5, 37° C.

FIG. 18. Testing turnover of Compound 4 in enhancing uncrosslinking of ATP aminal dimer. Dimer at high concentration (20 mM) was treated with Compound 4 (4 mM) or acetate buffer (20 mM) at 37° C., and loss of dimer followed by HPLC. The conversion seen at 72 h represents approximately three turnovers.

FIG. 19A-19B. (FIG. 19A) Proposed mechanism of action of Compound 4 in reversal of aminal crosslink, followed by reversal of hemiaminal adduct. Acid group acts as general acid to aid in breakdown of tetrahedral aminal and hemiaminal structures, while nucleophilic amino group aids in transimination. (FIG. 19B) Proposed transition state, in which negatively charged phosphonate binds and stabilizes formation of iminium, and proton transfer occurs. FIG. 20. PAGE gel showing retarded mobility of formaldehyde-crosslinked and adducted RNA, and effect of catalysts on reducing the crosslinks and retarded mobility. Formaldehyde-treated RNA was incubated at 37° C. for the time shown. U, untreated RNA; F, formaldehyde treated RNA; 0, 30 mM tris buffer pH 7, 1 mM EDTA; 1-3, 8 mM Compound 1, Compound 3, or Compound 4 in 30 mM tris buffer pH 7, 1 mM EDTA.

FIG. 21A-21C. Effect of heating on degradation of formaldehyde-treated RNA. (FIG. 21A) Representative MALDI mass spectrum of formaldehyde-treated RNA after 12 h at 60° C. in Tris buffer, showing numerous fragments from RNA degradation. (FIG. 21B) MALDI mass spectrum of formaldehyde-treated RNA after 12 h at 37° C. in Tris buffer, showing the effect of lower temperature on RNA stability. (FIG. 21C) Time course of RNA loss due to degradation, comparing heating at 60° C. in Tris buffer (*) to 37° C. incubations with buffer alone (*) and with 8 mM Compound 4 alone (*).

FIG. 22. Catalyst-enhanced removal of formaldehyde adducts from duplex DNA. A self-complementary DNA, (dGTTCTGCAGAAC)₂, isolated after 24-hour formaldehyde treatment was subjected to heating at 37° C. in 8 mM Tris buffer, pH 7.0 (*), or 8 mM Compound 4, pH 7.0 (*). The recovery of intact DNA was measured by mass spectrometry after 6 h and 18 h as a ratio relative to an internal untreated reference DNA peak. Error bars are standard deviations from two measurements of the same sample.

FIG. 23A-23B. Optimization of incubation temperature and time in catalyst-assisted RNA recovery from FFPE cell specimen as quantified by qRT-PCR. Shown is the threshold cycle (CO for detection of a 145-bp amplicon of the GAPDH mRNA transcript. 0.25 h, 80° C. are the recommended incubation conditions for the Qiagen AllPrep® DNA/RNA FFPE kit. Each incubation/extraction (20 mM catalyst—Compound 4) was performed once; mean and standard deviation of three replicates of qPCR quantification are shown. (FIG. 23A) Effect of varying incubation temperature. (FIG. 23B) Effect of varying incubation time at 55° C.

FIG. 24. Enhancement in recovery of RNA from formalin-fixed, paraffin-embedded cell specimens using Compound 4 (20 mM), relative to a commercial kit. Fold change in amplifiable RNA yield is plotted for eight amplicons (see FIG. 6 for primary data). Quantity (determined with a standard curve) is relative to a commercially-available kit (Qiagen AllPrep® DNA/RNA FFPE kit) which uses a spin column for isolation, and an 80° C., 0.25 h incubation step. The means of three independent experiments are shown, error bars indicating the standard deviation of variation in the qRT-PCR yield for each method relative to a fixed mean value for the kit. SC: spin column isolation. PCI: protocol of Masuda³ involving phenol-chloroform-isoamyl alcohol extraction followed by heating in buffer.

### DETAILED DESCRIPTION OF THE EMBODIMENTS

Methods are provided for reducing the number of adducts and/or crosslinks from biomolecules. In some cases, the biomolecules are aldehyde fixed biomolecules (biomolecules previously contacted with an aldehyde fixation reagent). Aspects of the methods include contacting a sample having fixed biomolecules (e.g., aldehyde fixed biomolecules) with an adduct reversal agent in an amount and for a period of time sufficient for reducing the number of fixation related adducts and/or crosslinks in the sample. For example, in some cases, subject methods include contacting a sample having aldehyde fixed biomolecules with an adduct reversal agent in an amount and for a period of time sufficient to reduce the number of aldehyde fixation related adducts and/or crosslinks in the sample. In some cases, the aldehyde is formaldehyde. As such, in some cases, subject methods include contacting a sample having formaldehyde fixed biomolecules with an adduct reversal agent in an amount and for a period of time sufficient to reduce the number of formaldehyde fixation related adducts and/or crosslinks in the sample.

In some cases the aldehyde fixed biomolecules are part of a biological sample. For example, in some cases, the aldehyde fixed biomolecules can be present in a cellular sample (e.g., a tissue sample) that was contacted with an aldehyde crosslinking (fixation) reagent (e.g., formaldehyde, glutaraldehyde, etc.). In some cases, subject methods include contacting a fixed biological sample (e.g., a tissue sample, e.g., a biopsy, a blood sample, etc.) with a subject adduct reversal agent. In some cases, a fixed biological sample is a formalin fixed paraffin embedded (FFPE) biological sample. As such, in some cases, subject methods include contacting a formaldehyde fixed biological sample (e.g., an FFPE biological sample) with an adduct reversal agent in an amount and for a period of time sufficient to reduce the number of formaldehyde fixation-related adducts and/or crosslinks in the sample.

Compositions and kits for practicing the methods of the disclosure are also provided. In some cases, the adduct reversal agent is a compound that includes an aromatic ring and at least one of: an amine group and a proton-donating group. In some cases, the adduct reversal agent is a compound that includes an aromatic ring, an amine group, and a proton-donating group. In some cases, the adduct reversal agent is a compound selected from the compounds of Table 1. In some cases, the adduct reversal agent is a compound selected from Compounds 1 to 4. In some cases, the adduct reversal agent is Compound 4.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

“Biomolecules” or “biological molecules” include proteins (e.g., soluble proteins, membrane-associated proteins, etc.), glycoproteins, ribonucleic acid (RNA), deoxyribonucleic acid (DNA), antibodies, lipids, carbohydrates, and molecules originating from pathogens.

“Buffering compounds” are used to produce a “buffer” (i.e., a buffering solution). Any convenient buffering compound can be used to produce a desired buffer (i.e., a buffer of any convenient pH). Examples of suitable buffering compounds include, but are not limited to: acetate, phosphate, PBS (phosphate-buffered-saline), citrate, borate, carbonate, bicarbonate, Tris [Tris(hydroxymethyl)amino methane], Tris-acetate-EDTA (ethylenediaminetetraacetic acid), Tris-borate, Tris-glycine, N,N-bis(2-hydroxyethyl)glycine(Bicine), N-tris(hydroxymethyl)methylglycine (Tricine), HEPES [4-(2-hydroxyethyl)piperazine-1-ethanesulfonic acid], MES [2-(N-morpholino)ethansulfonic acid], TAPS [N-tris(hydroxymethyl)-3-aminopropane sulfonic acid], 2-{[tris(hydroxymethyl)methyl]amino}ethanesulfonic acid (TES), MOPS [3-(N-morpholino) propanesulfonic acid], and any combination thereof. An “antigen retrieval (AR) buffer” is a solution that includes a buffer and which is used in retrieval of an antigen from a substrate, such as a tissue specimen. Antigen retrieval buffers are reviewed in Kim et al., Journal of Molecular Histology 35:409-416, 2004.

“Chelators” can include EDTA (ethylenediaminetetraacetic acid), potassium oxalate, calcein, DDAO [N,N-dimethyldecylamino-N-oxide], and DTPA [diethylenetriaminepentaacetic acid].

The term “chaotropic agent” refers to an agent, which, in aqueous solution and at a certain concentration, is capable of denaturing proteins. Chaotropic agents include, but are not limited to, sodium iodide, sodium perchlorate, sodium thiocyanate, potassium thiocyanate, guanidinium chloride, guanidinium thiocyanate, sodium trichloroacetate, and sodium trifluoroacetate.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “plurality” as used herein means greater than one. For example, a plurality can be 2 or more, 5 or more, 10 or more, 25 or more, 50 or more, 100 or more, 500 or more, 1,000 or more, 2,000 or more, 5,000 or more, 10⁴ or more, 10⁵ or more, 10⁶ or more, 10⁷ or more, etc.

### Methods and Compositions

Aspects of the disclosure include methods and compositions for removing adducts and/or crosslinks from biomolecules (e.g., fixed biomolecules). In some embodiments, subject methods include contacting fixed biomolecules (e.g., aldehyde fixed biomolecules such as formaldehyde fixed biomolecules) with an adduct reversal agent. In some cases, the biomolecules are part of a biological sample (e.g., an aldehyde fixed biological sample such as a formaldehyde fixed biological sample).

While the term “removal” when referring to the removal of adducts and/or crosslinks can include, but need not include, the complete removal of all adducts and/or crosslinks from a contacted sample (e.g., a sample that includes biomolecules). Thus, the term refers to the removal of at least some of the adducts and/or crosslinks from a contacted sample, thereby resulting in a sample of biomolecules having a reduced number of adducts and/or crosslinks compared to the number of adducts and/or crosslinks prior to contact with a subject adduct reversal agent. Thus, a subject method reduces the number of aldehyde fixation related adducts and/or crosslinks in a sample of biomolecules (e.g., a biological sample). The term “effective amount” is used herein to refer to an amount effective for removing adducts and/or crosslinks (e.g., reducing the number of adducts and/or crosslinks on biomolecules of a sample).

The term “fixing” or “fixation” as used herein is the process of preserving biological material (e.g., tissues, cells, organelles, molecules, etc.) from decay and/or degradation, by crosslinking the biomolecules. Fixation can include contacting a biomolecules (e.g., biomolecules of a biological sample, e.g., a cellular sample, a tissue sample, etc.; isolated biomolecules, etc.) with a fixation reagent (i.e., a reagent that contains at least one fixative). Such contact results in the addition of fixation-related adducts and/or crosslinks. Samples that include biomolecules (e.g, biological samples) can be contacted by a fixation reagent for a wide range of times, which can depend on the temperature, the nature of the sample, and on the fixative(s). For example, a biological sample can be contacted by a fixation reagent for 72 or less hours (e.g, 48 or less hours, 24 or less hours, 18 or less hours, 12 or less hours, 8 or less hours, 6 or less hours, 4 or less hours, 2 or less hours, 60 or less minutes, 45 or less minutes, 30 or less minutes, 25 or less minutes, 20 or less minutes, 15 or less minutes, 10 or less minutes, 5 or less minutes, or 2 or less minutes).

The biological molecules may be contained in, or recovered from, fixed samples of whole organs, organ substructures, surgical tissue biopsies, punch biopsies, fine-needle aspirate biopsies, bone, biological fluids, archival tissues, frozen tissue, tissue sections mounted on a substrate, such as a glass slide, etc.

The term “biological sample” encompasses blood and other liquid samples of biological origin, solid tissue samples such as a tissue sample (i.e., tissue specimen), a biopsy (i.e., a biopsy specimen), or tissue cultures or cells derived therefrom and the progeny thereof. The term also includes samples that have been manipulated in any way after their procurement, such as by treatment with reagents (e.g., fixation reagents, thereby generating a fixed biological sample); samples such as tissues that are embedded in medium (e.g., paraffin); sectioned tissue sample (e.g., sectioned samples that are mounted on a solid substrate such as a glass slide); washed; or enrichment for certain cell populations, such as cancer cells, neurons, stem cells, etc. The term also encompasses samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples (i.e., tissue specimens), organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample having cells (e.g., cancer cells) from a patient. A biological sample, by definition, includes biomolecules.

Biomolecules (e.g., biomolecules present in a biological sample), that have been contacted with a fixation reagent (e.g., a solution that includes a fixation reagent) are referred to herein as “fixed biomolecules.” For example, an aldehyde fixed biomolecule (e.g., a formaldehyde fixed biomolecule) has been contacted with an aldehyde fixation reagent (e.g., formaldehyde, glutaraldehyde). Thus, the term “fixed biomolecules” refers to a collection of biomolecules (i.e., a plurality of biomolecules, e.g., such as exists in a biological sample such as a blood sample, a tissue sample, etc.) where one or more of the biomolecules has one or more adducts from a fixation agent (e.g., an aldehyde fixation agent). In some cases, the aldehyde fixation agent is formaldehyde. Aldehyde fixed biomolecules (e.g., formaldehyde fixed biomolecules) in a sample include biomolecules (e.g., RNA molecules, DNA molecules, and/or protein molecules) having fixation-related adducts. In some cases, the aldehyde fixed biomolecules (e.g., formaldehyde fixed biomolecules) include biomolecules (e.g., RNA molecules, DNA molecules, and/or protein molecules) that are crosslinked (i.e., biomolecules having fixation-related crosslinks). In some cases, the aldehyde fixed biomolecules include a combination of (i) RNA molecules having adducts, DNA molecules having adducts, and/or protein molecules having adducts; and (ii) crosslinked RNA molecules, crosslinked DNA molecules, and/or crosslinked proteins. In some cases, the aldehyde fixed biomolecules include: biomolecules that were fixed with formaldehyde, biomolecules that were fixed with glutaraldehyde, or biomolecules that were fixed with a combination of formaldehyde and glutaraldehyde. For example, suitable aldehyde fixed biomolecules include biomolecules having: formaldehyde crosslinks and/or adducts, glutaraldehyde crosslinks and/or adducts, or a combination thereof.

The term “fixed biological sample” is used herein to refer to a biological sample (e.g., a tissue sample, a blood sample, a biopsy, etc.) that has been contacted with a fixation reagent. For example, an aldehyde fixed biological sample (e.g., a formaldehyde fixed biological sample) has been contacted with an aldehyde fixation reagent (e.g., formaldehyde, glutaraldehyde). Thus, the term “fixed biological sample” refers to a biological (e.g., a blood sample, a tissue sample, a tissue section, a biopsy, an aspirate, etc.) where one or more of the biomolecules of the sample has one or more adducts and/or crosslinks from a fixation agent (e.g., an aldehyde fixation agent). The adducts and crosslinks can be referred to as “fixation-related adducts” and “fixation related crosslinks.” In some cases, the aldehyde fixation agent is formaldehyde. Aldehyde fixed biological samples (e.g., formaldehyde fixed biological samples) include biomolecules (e.g., RNA molecules, DNA molecules, and/or protein molecules) having fixation-related adducts and/or crosslinks.

Common fixation reagents include crosslinking fixatives, precipitating fixatives, oxidizing fixatives, mercurials, and the like. Crosslinking fixatives chemically join two or more molecules by a covalent bond and a wide range of cross-linking reagents can be used. Examples of crosslinking fixatives include but are not limited to aldehyde fixatives (e.g., formaldehyde, also commonly referred to as “paraformaldehyde” and “formalin”; glutaraldehyde; etc.), imidoesters, NHS (N-Hydroxysuccinimide) esters, and the like.

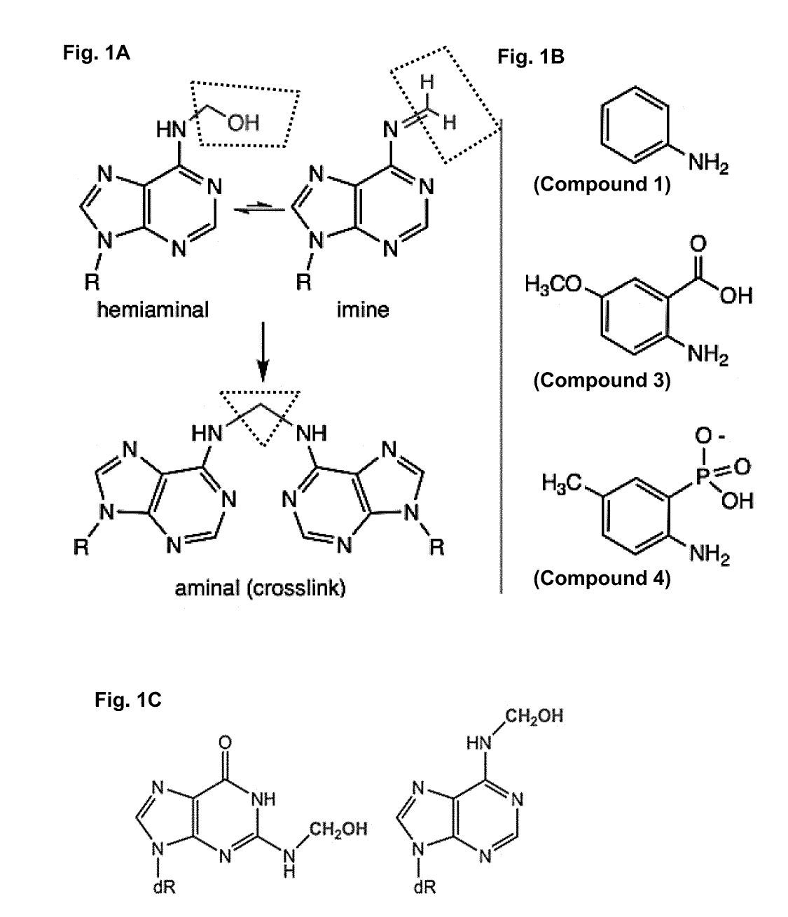

Contact between an aldehyde fixation agent and biomolecules results in the addition of adducts and/or crosslinks to the biomolecules. For example, when a biomolecule such as an RNA or DNA is contacted with formaldehyde, The RNA/DNA bases of the molecule can acquire an adduct (FIG. 1C). For example, as depicted in FIG. 1A-1C, addiction of an adduct can result in a hemiaminal (also termed methylol), which can convert to an imine via dehydration. The imine can later react with another DNA or RNA base to form a crosslink (an aminal) in which the two bases are crosslinked via a methylene group from the formaldehyde (FIG. 1A-1C). The presence of imine and hemiaminal adducts, and/or aminal crosslinks, reduces the ability to detect (e.g., bind to, amplify, sequence, hybridize to) the fixed biomolecules (e.g., proteins, RNA, DNA). Attempts to remove these adducts and/or crosslinks by standard methods can cause further damage (loss of bases, chain hydrolysis, cleavage, denaturation, etc.) due to heating and/or buffer conditions. Consequences of tissue fixation and the benefits of removing adducts and/or crosslinks are described in U.S. Pat. No. 8,288,122, which is hereby incorporated by reference in its entirety.

For example, formaldehyde fixes proteins in tissue by cross-linking basic amino acids, such as lysine and glutamine, and through the formation of methylol adducts with these basic amino acids. Both intra-molecular and inter-molecular cross-links are formed. These cross-links preserve protein secondary structure while destroying enzyme activity by forming active-site adducts, which prevent enzyme conformational changes and inhibit diffusion of both enzyme and substrate through the cellular matrix. Formaldehyde reacts with amino groups (such as the amino group of Lys) to form reactive methylol compounds. Under suitable steric conditions, the reactive methylol compounds condense with amine, amide, phenol, indole, and imidazole side chains to form methylene bridges that cross-link polypeptide chains. These reactions have discouraged investigators from using archival tissue specimens (e.g., clinical biological samples including FFPE archival tissues) for protein analysis. For example, two-dimensional (2-D) gel electrophoresis requires that protein molecules are solubilized to be loaded on the gel and separated by molecular weight during electrophoresis. Furthermore, 2-D separation requires protein ionization for successful isoelectric focusing. The formation of either methylol adducts or methylene cross-links neutralizes basic amines, which significantly perturbs the isoelectric focusing step.

Another unwanted effect of formaldehyde fixation is a reduction of immunohistochemical reactivity in tissue sections. This loss of reactivity is believed to arise from chemical epitope modification and the inability of antibodies to diffuse into the cross-linked tissue. These effects may be partially reversed by exposure of fixed tissue sections to high temperatures for short periods of time in the presence of aqueous salt or protein denaturant solutions. Shi et al. (J. Histochem. Cytochem. 39:741-748, 1991) have demonstrated that heat treatment can improve retrieval of antigens (i.e., “antigen retrieval”, “unmasking”) masked by formalin fixation and that optimal results are correlated with the product of the heating temperature and the time of heat treatment (CAP Today 9:116-123, 1995).

In some embodiments, the fixative (fixation reagent) is formaldehyde. It would be readily understood by one of ordinary skill in the art that the term formaldehyde (when referring to formaldehyde as a fixative) is also referred to in the art as “paraformaldehyde” and “formalin”, both of which are terms with specific meanings related to the formaldehyde composition (e.g., formalin is a mixture of formaldehyde and methanol). As such, formaldehyde fixed biomolecules (e.g., a fixed tissue) would have the same type of adducts and/or crosslinks as formalin fixed biomolecules.

Fixation of biomolecules using formaldehyde is a well known and common practice. An example of a suitable final concentration of formaldehyde in a fixation reagent is 0.1 to 10%, 1-8%, 1-4%, 1-2%, 3-5%, or 3.5-4.5%. In some cases, biomolecules are fixed in a final concentration of 4% formaldehyde (as diluted from a more concentrated stock solution, e.g., 38%, 37%, 36%, 20%, 18%, 16%, 14%, 10%, 8%, 6%, etc.). In some cases, biomolecules are fixed in a final concentration of 10% formaldehyde. In some cases biomolecules are fixed in a final concentration of 1% formaldehyde. In some cases, an aldehyde fixative is glutaraldehyde. A suitable concentration of glutaraldehyde in a fixation reagent is 0.1 to 1%. A fixation reagent can contain more than one fixative in any combination. For example, in some cases biomolecules (e.g., biological samples such as tissue specimens) are contacted with a fixation reagent containing both formaldehyde and glutaraldehyde, and thus the contacted biomolecules can include fixation related adducts and/or crosslinks that include formaldehyde fixation related adducts and/or crosslinks as well as glutaraldehyde fixation related adducts and/or crosslinks.

In some cases aldehyde fixed biomolecules are present in a biological sample that is embedded. For example, in some cases, the biological sample is a tissue sample that has been embedded in a convenient media. Examples of embedding mediums include but are not limited to: paraffin, paraffin-containing compounds, araldite, celloidin, DURCUPAN™ embedding medium, epoxy, glycol methacrylate, hydroxypropyl methacrylate, JB-4™ embedding medium, Spurr, or LR WHITE™ embedding medium. The fixed specimens have been fixed with a variety of cross-linking agents, such as formaldehyde, paraformaldehyde, glutaraldehyde, or 1-ethyl-3-(3-dimethylaminopropyl).

In some cases when the aldehyde fixed biomolecules are present in a biological sample that is embedded (e.g., embedded in paraffin, e.g., an FFPE biological sample), the embedding medium can be removed from the biologic sample and./or the biological sample can be rehydrated prior to contacting the sample with a subject adduct reversal agent. For example, if the biological sample is embedded in paraffin, the sample can be deparaffinized and/or rehydrated prior to contacting the sample with a subject adduct reversal agent. Any convenient method can be used for deparaffinization and/or rehydration. In some cases, the embedding medium is not removed prior to contact with a subject adduct reversal agent. For example, in some cases, the biological sample is not deparaffinized prior to contact with a subject adduct reversal agent. In some cases, a subject method includes a step of removing an embedding medium (e.g., paraffin) from a biological sample. In some cases, a subject method includes a step of removing an embedding medium (e.g., paraffin) from an aldehyde fixed biological sample (e.g., a formaldehyde fixed biological sample, and FFPE biological sample) prior to contacting the sample with a subject adduct reversal agent. In some cases, a subject method includes a step of removing an embedding medium (e.g., paraffin) from a biological sample simultaneous with contacting the sample with a subject adduct reversal agent. In some cases, a subject method includes a step of removing an embedding medium (e.g., paraffin) from a biological sample after contacting the sample with a subject adduct reversal agent.

In some cases, the methods include a step of contacting the sample having aldehyde fixed biomolecules (e.g., a sample having formaldehyde fixed biomolecules, an aldehyde fixed biological sample, a formaldehyde fixed biological sample, and FFPE biological sample, etc.) with a proteolytic enzyme (e.g., trypsin, chymotrypsin, proteinase K, papain, pepsin, pronase, endoproteinase Lys-C, endoproteinase glu-C, and the like). The terms “proteolytic enzyme” and “protease” are used interchangeably herein. For example, in some cases, the methods include a step of contacting an aldehyde fixed biological sample, (e.g., a formaldehyde fixed biological sample, an FFPE biological sample, etc.) with a proteolytic enzyme (i.e., a protease) (e.g., trypsin, chymotrypsin, proteinase K, papain, pepsin, pronase, endoproteinase Lys-C, endoproteinase glu-C, and the like). Any convenient protocol can be used for proteolytic digestion (e.g., contacting the biological sample with a proteolytic enzyme), and such methods would be readily available to one of ordinary skill in the art.

Contact with a proteolytic enzyme can be prior to, simultaneously with, or after contacting the sample with a subject adduct reversal agent. Thus, in some cases, a subject method includes a step of contacting an aldehyde fixed biological sample (e.g., a formaldehyde fixed biological sample, and FFPE biological sample) with a proteolytic enzyme (e.g., trypsin, chymotrypsin, proteinase K, papain, pepsin, pronase, endoproteinase Lys-C, endoproteinase glu-C, and the like) prior to contacting the sample with a subject adduct reversal agent. In some cases, a subject method includes a step of contacting a biological sample with a proteolytic enzyme (e.g., trypsin, chymotrypsin, proteinase K, papain, pepsin, pronase, endoproteinase Lys-C, endoproteinase glu-C, and the like) simultaneous with contacting the sample with a subject adduct reversal agent. In some cases, a subject method includes a step of contacting a biological sample with a proteolytic enzyme (e.g., trypsin, chymotrypsin, proteinase K, papain, pepsin, pronase, endoproteinase Lys-C, endoproteinase glu-C, and the like) after contacting the sample with a subject adduct reversal agent.

In some cases, a sample having aldehyde fixed biomolecules (e.g., a sample having formaldehyde fixed biomolecules, an aldehyde fixed biological sample, a formaldehyde fixed biological ample, an FFPE biological sample, and the like) is contacted with an adduct reversal agent in a solution buffered to a pH in a range of from 4 to 8.5 (e.g., in a range of from 4 to 8, in a range of from 4 to 7.5, in a range of from 4 to 7, in a range of from 4 to 6.5, in a range of from 4 to 6, in a range of from 4.5 to 8, in a range of from 4.5 to 7.5, in a range of from 4.5 to 7, in a range of from 4.5 to 6.5, in a range of from 4.5 to 6, in a range of from 5 to 8, in a range of from 5 to 7.5, in a range of from 5 to 7, in a range of from 5 to 6.5, in a range of from 5 to 6, in a range of from 6 to 8, in a range of from 6.5 to 7.5, at a pH of 4, at a pH of 4.2, at a pH of 4.5, at a pH of 4.8, at a pH of 5, at a pH of 5.5, at a pH of 6, at a pH of 6.5, at a pH of 6.8, at a pH of 7, at a pH of 7.2, etc.). In some cases, the sample having the aldehyde fixed biomolecules (e.g., a sample having formaldehyde fixed biomolecules, an aldehyde fixed biological sample, a formaldehyde fixed biological ample, an FFPE biological sample, and the like) is contacted with an adduct reversal agent in a solution buffered to a pH that is at or near the pKa of the adduct reversal agent. In some cases (e.g., in a case where a subject adduct reversal agent has an acidic pKa, e.g., Compounds 9 to 12 of Table 1), the sample having the aldehyde fixed biomolecules (e.g., a sample having formaldehyde fixed biomolecules, an aldehyde fixed biological sample, a formaldehyde fixed biological ample, an FFPE biological sample, and the like) is contacted with an adduct reversal agent in a solution buffered to a pH in a range of from 4 to 6.5 (e.g, in a range of from 4.5 to 6, in a range of from 4.5 to 5.5, in a range of from 5 to 6.5, in a range of from 4 to 6, in a range of from 4 to 5.5, in a range of from 4 to 5, in a range of from 4 to 4.5, in a range of from 4.2 to 4.8, at a pH of 4, at a pH of 4.2, at a pH of 4.5, at a pH of 4.8, at a pH of 5, at a pH of 5.5, etc.).

In some cases, a sample having aldehyde fixed biomolecules (e.g., a sample having formaldehyde fixed biomolecules, an aldehyde fixed biological sample, a formaldehyde fixed biological ample, an FFPE biological sample, and the like) is contacted with a subject adduct reversal agent for a period of time in a range of from 20 minutes to 48 hours (e.g., from 20 minutes to 48 hours, from 20 minutes to 36 hours, from 20 minutes to 24 hours, from 20 minutes to 18 hours, from 20 minutes to 12 hours, from 20 minutes to 10 hours, from 20 minutes to 6 hours, from 20 minutes to 4 hours, from 20 minutes to 3 hours, from 20 minutes to 2 hours, from 20 minutes to 1 hour, from 30 minutes to 48 hours, from 30 minutes to 36 hours, from 30 minutes to 24 hours, from 30 minutes to 18 hours, from 30 minutes to 12 hours, from 30 minutes to 10 hours, from 30 minutes to 6 hours, from 30 minutes to 4 hours, from 30 minutes to 3 hours, from 30 minutes to 2 hours, from 30 minutes to 1 hour, from 45 minutes to 48 hours, from 45 minutes to 36 hours, from 45 minutes to 24 hours, from 45 minutes to 18 hours, from 45 minutes to 12 hours, from 45 minutes to 10 hours, from 45 minutes to 6 hours, from 45 minutes to 4 hours, from 45 minutes to 3 hours, from 45 minutes to 2 hours, from 45 minutes to 1 hour, from 1 hour to 48 hours, from 1 hour to 36 hours, from 1 hour to 24 hours, from 1 hour to 18 hours, from 1 hour to 12 hours, from 1 hour to 10 hours, from 1 hour to 6 hours, from 1 hour to 4 hours, from 1 hour to 3 hours, from 1 hour to 2 hours, from 2 hours to 48 hours, from 2 hours to 36 hours, from 2 hours to 24 hours, from 2 hours to 18 hours, from 2 hours to 12 hours, from 2 hours to 10 hours, from 2 hours to 6 hours, from 2 hours to 4 hours, or from 2 hours to 3 hours). In some cases, a sample having aldehyde fixed biomolecules (e.g., a sample having formaldehyde fixed biomolecules, an aldehyde fixed biological sample, a formaldehyde fixed biological ample, an FFPE biological sample, and the like) is contacted with a subject adduct reversal agent for at least 20 minutes (e.g., at least 30 minutes, at least 40 minutes, at least 1 hour, at least 1.5 hours, at least 2 hours, at least 3 hours, at least 4 hours, etc.).

In some cases, a sample having the aldehyde fixed biomolecules (e.g., a sample having formaldehyde fixed biomolecules, an aldehyde fixed biological sample, a formaldehyde fixed biological ample, an FFPE biological sample, and the like) is contacted with an adduct reversal agent at a temperature in a range of from 15° C. to 80° C. (e.g., from 15° C. to 75° C., 15° C. to 70° C., 15° C. to 65° C., 15° C. to 60° C., 15° C. to 55° C., 15° C. to 50° C., 15° C. to 45° C., from 15° C. to 40° C., from 15° C. to 37° C., from 15° C. to 35° C., from 15° C. to 30° C., from 20° C. to 45° C., from 20° C. to 40° C., from 20° C. to 37° C., from 20° C. to 35° C., from 20° C. to 30° C., from 22° C. to 28° C., at a temperature of 25° C.), or is contacted with an adduct reversal agent at room temperature.

A subject adduct reversal agent can catalyze the removal of adducts and/or crosslinks under relatively mild conditions (conditions that are not as damaging to biological samples, such as tissue samples, as the conditions used by other methods of crosslink removal). Thus, the subject methods result in less damage to biomolecules than other available methods for removing adducts and/or crosslinks. For example, the subject methods can result in much less damage to biomolecules than other available methods for removing adducts and/or crosslinks. Other methods use high temperatures and are carried out at pHs that are damaging to biomolecules such as nucleic acids and proteins. Thus, biomolecules can be “recovered” intact from aldehyde fixed samples using the subject methods. Because the subject methods can be carried out in relatively mild conditions (pH near neutral, temperatures near room temperature, short time frame of treatment, etc.), and inflict less damage on biomolecules present in aldehyde fixed samples, downstream methods can be performed to detect biomolecules present in the sample after the number of adducts and/or crosslinks have been reduced. For example, because the subject methods can be carried out in relatively mild conditions (pH near neutral, temperatures near room temperature, short time frame of treatment, etc.), and can inflict much less damage on biomolecules present in aldehyde fixed samples, downstream methods can be performed to detect biomolecules present in the sample after the number of adducts and/or crosslinks have been reduced. Thus, in some embodiments, the methods include, after contacting a sample having aldehyde fixed biomolecules (e.g., a sample having formaldehyde fixed biomolecules, an aldehyde fixed biological sample, a formaldehyde fixed biological ample, an FFPE biological sample, and the like) with an adduct reversal agent, a step of detecting a biomolecule (e.g., RNA, DNA, and/or protein) in the biological sample. Any convenient detecting method can be used. In some cases, said detecting includes PCR, nucleic acid sequencing, in situ hybridization, and/or a protein detection method (e.g., an enzymatic detection assay, an antibody-based protein detection method, western blot, enzyme-linked immunosorbent assay (ELISA), immunohistology, an immunoenzymatic assay, immunofluorescence detection, fluorescent dye-based quantification (e.g. Qubit), spectroscopic quantification (e.g. Nanodrop) etc.)

### Adduct Reversal Agent.

A subject adduct reversal agent is an agent that catalyzes the removal of adducts and/or crosslinks from fixed biomolecules (e.g, aldehyde fixed biomolecules, formaldehyde fixed biomolecules), e.g., upon contact with a sample having aldehyde fixed biomolecules (e.g., a sample having formaldehyde fixed biomolecules, an aldehyde fixed biological sample, a formaldehyde fixed biological ample, an FFPE biological sample, and the like). Because a crosslink is a form of adduct, an adduct reversal agent can be said to remove crosslinks as well as other adducts (e.g., aldehyde fixation related adducts; formaldehyde fixation related adducts, e.g., a methylol, an imine; and the like). Thus, an adduct reversal agent can be said to remove adducts and/or crosslinks. In some cases, the pKa of the adduct reversal agent is in a range of from 4 to 8.5 (e.g., in a range of from 4 to 8, in a range of from 4 to 7.5, in a range of from 4 to 7, in a range of from 4 to 6.5, in a range of from 4 to 6, in a range of from 4.5 to 8, in a range of from 4.5 to 7.5, in a range of from 4.5 to 7, in a range of from 4.5 to 6.5, in a range of from 4.5 to 6, in a range of from 5 to 8, in a range of from 5 to 7.5, in a range of from 5 to 7, in a range of from 5 to 6.5, in a range of from 5 to 6, in a range of from 6 to 8, in a range of from 6.5 to 7.5, 4, 4.2, 4.5, 4.8, 5, 5.5, 6, 6.5, 6.8, 7, 7.2, 7.4, etc.).

In some embodiments, the adduct reversal agent is a compound that includes an amine group and/or a proton-donating group. In some cases, the adduct reversal agent is a compound having an aromatic ring and an amine group (e.g., suitable amine groups include but are not limited to: primary amines, secondary amines, aromatic amines, etc). From Table 1, examples of such compounds are Compounds 1-5, 7-8, and 12-20. As such, in some cases, the adduct reversal agent is selected from Compounds 1-5, 7-8, and 12-20 of Table 1. In some cases, a subject method includes contacting a sample having aldehyde fixed biomolecules (e.g., formaldehyde fixed biomolecules) with at least one compound (adduct reversal agent) selected from: Compound 1 [Aniline], Compound 2 [2-aminobenzoic acid], Compound 3 [2-amino-5-methoxybenzoic acid], Compound 4 [(2-amino-5-methylphenyl) phosphonic acid], Compound 5 [4-aminobenzoic acid], Compound 7 [3,5-diaminobenzoic acid], Compound 8 [benzene-1,3-diamine], Compound 12 [2-aminobenzene-1-sulfonic acid], Compound 13 [8-amino-4-nitronaphthalen-1-ol], Compound 14 [3-amino-7-nitro-1H-indol-4-ol], Compound 15 [(2-hydrazinylphenyl) phosphonic acid], Compound 16 [2-amino-4-nitrophenol], Compound 17 [1H-1,3-benzodiazol-2-ylmethanamine], Compound 18 [6-amino-2H-1,3-benzodioxole-5-carboxylic acid], Compound 19 [(6-amino-2H-1,3-benzodioxol-5-yl) phosphonic acid], and Compound 20 [2-(1H-imidazol-4-yl)-4-methoxyaniline].

In some embodiments, the adduct reversal agent is a compound having an aromatic ring (e.g., a primary amine, a secondary amine, an aromatic amine, etc.) and a proton-donating group. Suitable proton-donating groups include, but are not limited to: a carboxylic acid group, a phosphoric acid group, a phosphonic acid group, a sulfuric acid group (sulfonic acid), a nitric acid group (nitronic acid, a nitro group, etc.), a phosphonamidic acid group, a phenol group, a tetrazole group, a benzimidazolidinium group, a hydroxy-benzotriazole group, a hydroxamic acid group, a boronic acid group, etc. In some cases, the pKa of the proton-donating group is in a range of from 4 to 8.5 (e.g., in a range of from 4 to 8, in a range of from 4 to 7.5, in a range of from 4 to 7, in a range of from 4 to 6.5, in a range of from 4 to 6, in a range of from 4.5 to 8, in a range of from 4.5 to 7.5, in a range of from 4.5 to 7, in a range of from 4.5 to 6.5, in a range of from 4.5 to 6, in a range of from 5 to 8, in a range of from 5 to 7.5, in a range of from 5 to 7, in a range of from 5 to 6.5, in a range of from 5 to 6, in a range of from 6 to 8, in a range of from 6.5 to 7.5, 4, 4.2, 4.5, 4.8, 5, 5.5, 6, 6.5, 6.8, 7, 7.2, 7.4, etc.). From Table 1, examples of compounds that have an aromatic ring and a proton-donating group are Compounds 2-7, and 9-19. As such, in some cases, the adduct reversal agent is selected from Compounds 2-7, and 9-19 of Table 1. In some cases, a subject method includes contacting a sample having aldehyde fixed biomolecules (e.g., formaldehyde fixed biomolecules) with at least one compound (adduct reversal agent) selected from: Compound 2 [2-aminobenzoic acid], Compound 3 [2-amino-5-methoxybenzoic acid], Compound 4 [(2-amino-5-methylphenyl) phosphonic acid], Compound 5 [4-aminobenzoic acid], Compound 6 [benzoic acid], Compound 7 [3,5-diaminobenzoic acid], Compound 9 [benzenesulfonic acid], Compound 10 [phenylphosphonic acid], Compound 11 [(3-methylphenyl)phosphonic acid], Compound 12 [2-aminobenzene-1-sulfonic acid], Compound 13 [8-amino-4-nitronaphthalen-1-ol], Compound 14 [3-amino-7-nitro-1H-indol-4-01], Compound 15 [(2-hydrazinylphenyl) phosphonic acid], Compound 16 [2-amino-4-nitrophenol], Compound 17 [1H-1,3-benzodiazol-2-ylmethanamine], Compound 18 [6-amino-2H-1,3-benzodioxole-5-carboxylic acid], and Compound 19 [(6-amino-2H-1,3-benzodioxol-5-yl) phosphonic acid].

In some embodiments, the adduct reversal agent is a compound having an aromatic ring, an amine group (e.g., a primary amine, a secondary amine, an aromatic amine, etc.), and a proton-donating group (see above for examples of suitable proton-donating groups). From Table 1, examples of such a compound are Compounds 2-5, 7, 12-16, and 18-19. As such, in some cases, the adduct reversal agent is selected from Compounds 2-5, 7, 12-16, and 18-19 of Table 1. In some cases, a subject method includes contacting a sample having aldehyde fixed biomolecules (e.g., formaldehyde fixed biomolecules) with at least one compound (adduct reversal agent) selected from: Compound 2 [2-aminobenzoic acid], Compound 3 [2-amino-5-methoxybenzoic acid], Compound 4 [(2-amino-5-methylphenyl) phosphonic acid], Compound 5 [4-aminobenzoic acid], Compound 7 [3,5-diaminobenzoic acid], Compound 12 [2-aminobenzene-1-sulfonic acid], Compound 13 [8-amino-4-nitronaphthalen-1-ol], Compound 14 [3-amino-7-nitro-1H-indol-4-ol], Compound 15 [(2-hydrazinylphenyl) phosphonic acid], Compound 16 [2-amino-4-nitrophenol], Compound 18 [6-amino-2H-1,3-benzodioxole-5-carboxylic acid], and Compound 19 [(6-amino-2H-1,3-benzodioxol-5-yl) phosphonic acid].

In some embodiments, the adduct reversal agent is an aminobenzene having an ortho phosphonate group. From Table 1, examples of such a compound include Compounds 4, 15, and 19. As such, in some cases, the adduct reversal agent is selected from Compounds 4, 15, and 19 of Table 1. In some cases, a subject method includes contacting a sample having aldehyde fixed biomolecules (e.g., formaldehyde fixed biomolecules) with at least one compound (adduct reversal agent) selected from: Compound 4 [(2-amino-5-methylphenyl) phosphonic acid], Compound 15 [(2-hydrazinylphenyl) phosphonic acid], and Compound 19 [(6-amino-2H-1,3-benzodioxol-5-yl) phosphonic acid].

In some cases, a subject method includes contacting a sample having aldehyde fixed biomolecules (e.g., formaldehyde fixed biomolecules) with at least one compound selected from Table 1. As such, in some cases, the adduct reversal agent is selected from Table 1. In some cases, a subject method includes contacting a sample having aldehyde fixed biomolecules (e.g., formaldehyde fixed biomolecules) with at least one compound (adduct reversal agent) selected from: Compound 1 [Aniline], Compound 2 [2-aminobenzoic acid], Compound 3 [2-amino-5-methoxybenzoic acid], Compound 4 [(2-amino-5-methylphenyl) phosphonic acid], Compound 5 [4-aminobenzoic acid], Compound 6 [benzoic acid], Compound 7 [3,5-diaminobenzoic acid], Compound 8 [benzene-1,3-diamine], Compound 9 [benzenesulfonic acid], Compound 10 [phenylphosphonic acid], Compound 11 [(3-methylphenyl)phosphonic acid], Compound 12 [2-aminobenzene-1-sulfonic acid], Compound 13 [8-amino-4-nitronaphthalen-1-ol], Compound 14 [3-amino-7-nitro-1H-indol-4-ol], Compound 15 [(2-hydrazinylphenyl) phosphonic acid], Compound 16 [2-amino-4-nitrophenol], Compound 17 [1H-1,3-benzodiazol-2-ylmethanamine], Compound 18 [6-amino-2H-1,3-benzodioxole-5-carboxylic acid], Compound 19 [(6-amino-2H-1,3-benzodioxol-5-yl) phosphonic acid], and Compound 20 [2-(1H-imidazol-4-yl)-4-methoxyaniline].

In some cases, a subject method includes contacting a sample having aldehyde fixed biomolecules (e.g., formaldehyde fixed biomolecules) with at least one compound selected from Compounds 1-12 of Table 1. As such, in some cases, the adduct reversal agent is selected from Compounds 1-12 of Table 1. In some cases, a subject method includes contacting a sample having aldehyde fixed biomolecules (e.g., formaldehyde fixed biomolecules) with at least one compound (adduct reversal agent) selected from: Compound 1 [Aniline], Compound 2 [2-aminobenzoic acid], Compound 3 [2-amino-5-methoxybenzoic acid], Compound 4 [(2-amino-5-methylphenyl) phosphonic acid], Compound 5 [4-aminobenzoic acid], Compound 6 [benzoic acid], Compound 7 [3,5-diaminobenzoic acid], Compound 8 [benzene-1,3-diamine], Compound 9 [benzenesulfonic acid], Compound 10 [phenylphosphonic acid], Compound 11 [(3-methylphenyl)phosphonic acid], and Compound 12 [2-aminobenzene-1-sulfonic acid].

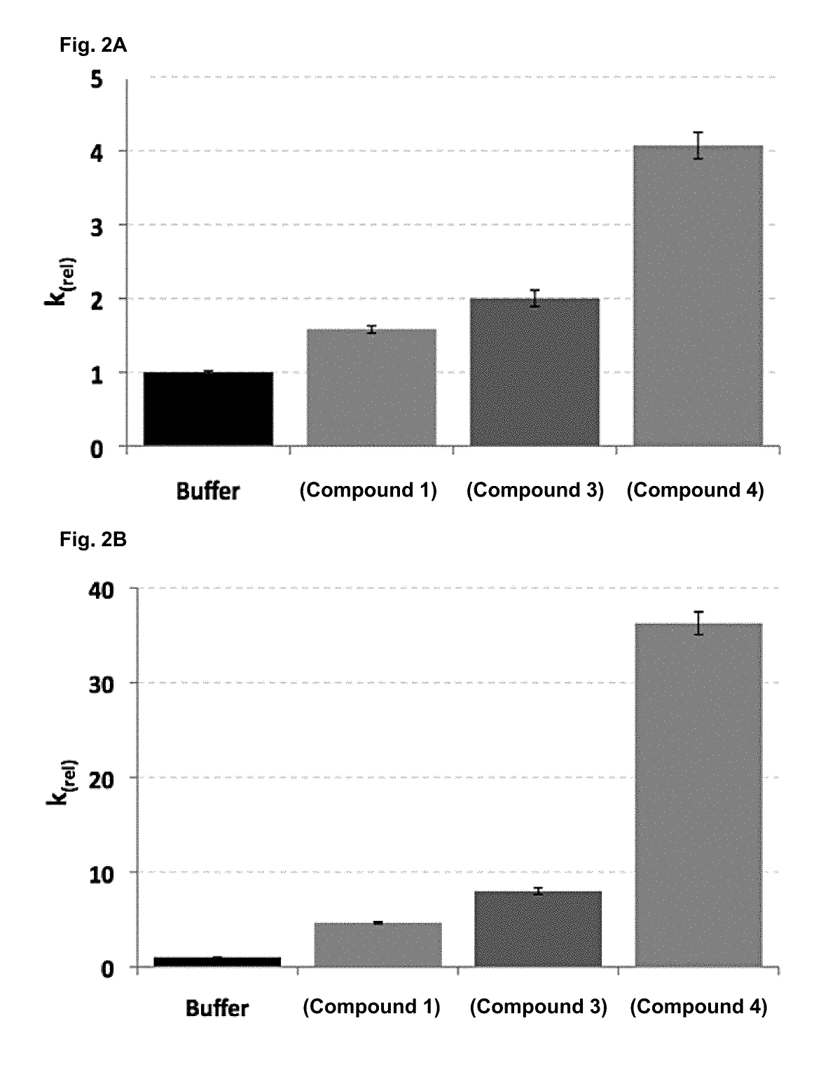

In order to determine whether a compound (or a composition comprising one or more compounds) of interest functions as an adduct reversal agent (e.g., a compound that can be used in the subject methods), the compound can be tested, at a temperature and pH suitable for use as a subject adduct reversal agent, for its ability to remove adducts and/or crosslinks from biomolecules, e.g., compared to buffer alone, compared to a compound known to be a suitable adduct reversal agent, compared to a compound known not to be a suitable adduct reversal agent, and/or compared to a known standard reference value. Any convenient assay can be used to test for the presence of adducts and/or crosslinks (e.g., hemiaminals, imines, and/or aminals). For example, the examples section below provides assays that can be used to determine whether adducts and/or crosslinks have been removed. Such assays can be carried over time, at a variety of temperatures and pH values to determine whether a candidate agent is suitable to reduce the amount of adducts and/or crosslinks in a sample having biomolecules. For example, a candidate adduct reversal agent can be tested for function in assays such as those shown in FIGS. 2-24 (e.g., FIG. 2A-FIG. 24).

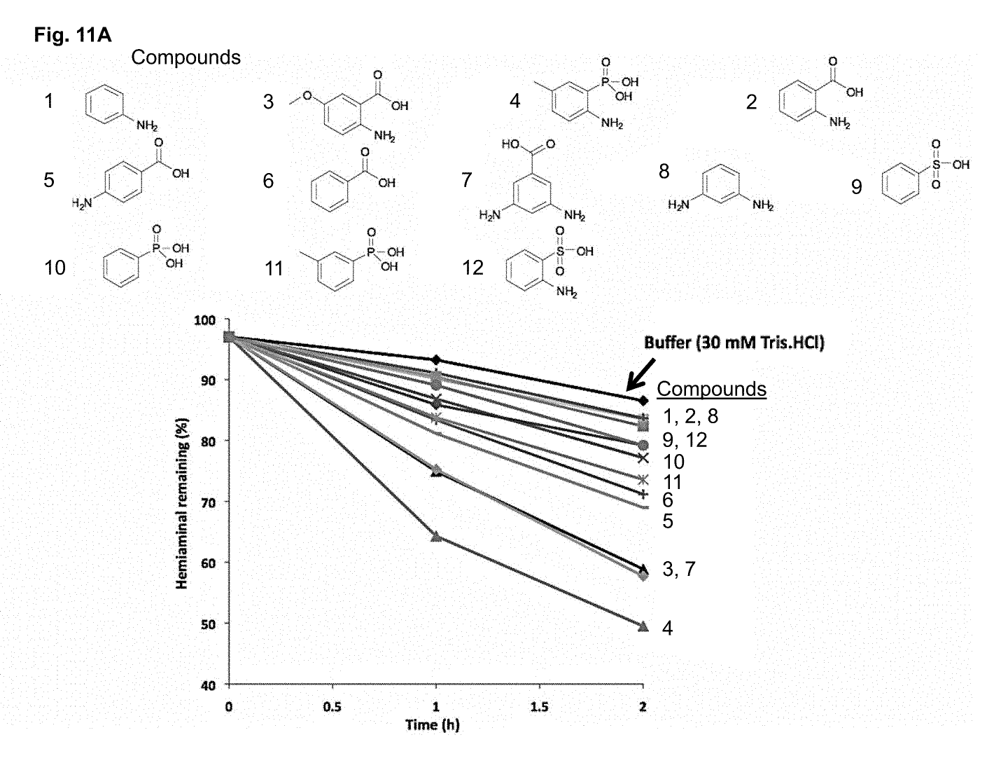

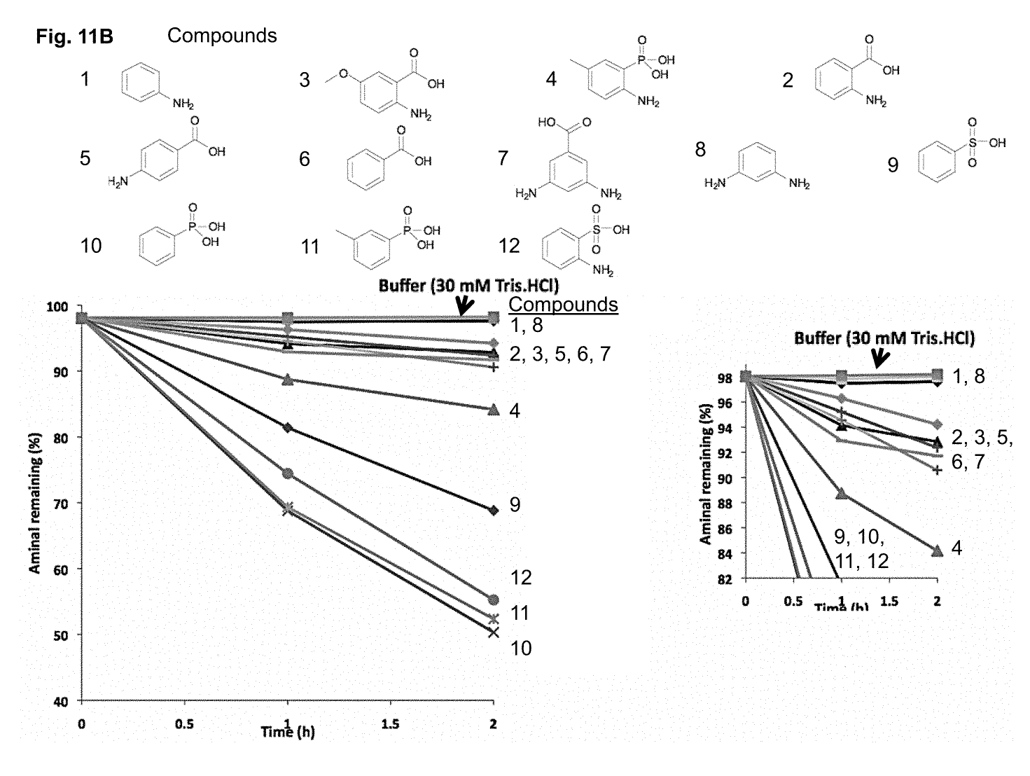

In some cases, such an assay can include measuring the percent hemiaminal remaining in a sample after a particular time of incubation (e.g., 2 hours, FIG. 11A). In some cases, such an assay can include measuring the percent aminal remaining in a sample after a particular time of incubation (e.g., 2 hours, FIG. 11B).

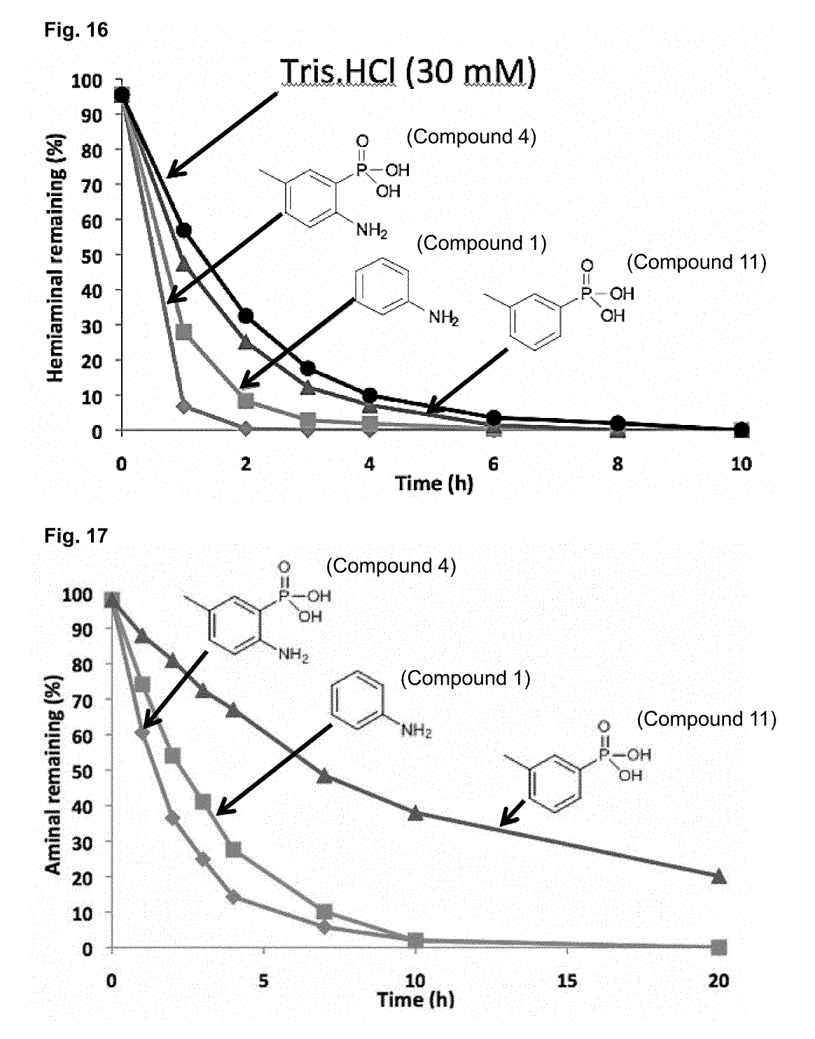

In some cases, a subject adduct reversal agent is one in which, upon contacting a sample having formaldehyde fixed biomolecules (with the adduct reversal agent at a concentration in a range of from 15 mM to 20 mM), for 2 hours at 37° C., at pH 7, 30% or less (e.g., 25% or less, 20% or less, 15% or less, 10% or less, 5% or less, or 2% or less) of hemiaminals remain compared to the amount of hemiaminals prior to contact (see FIG. 16). In some cases, a subject adduct reversal agent is one in which, upon contacting a sample having formaldehyde fixed biomolecules (with the adduct reversal agent at a concentration in a range of from 15 mM to 20 mM), for 4 hours at 37° C., at pH 4.5, 80% or less (e.g., 70% or less, 60% or less, 50% or less, 40% or less, or 20% or less) of aminals remain compared to the amount of aminals prior to contact (see FIG. 17). In some cases, a subject adduct reversal agent is one in which, upon contacting a sample having formaldehyde fixed biomolecules (with the adduct reversal agent at a concentration in a range of from 15 mM to 20 mM), for 10 hours at 37° C., at pH 4.5, 50% or less (e.g., 40% or less, 30% or less, 25% or less, 20% or less, 15% or less, 10% or less, or 5% or less) of aminals remain compared to the amount of aminals prior to contact (see FIG. 17).

In some cases, a subject adduct reversal agent is one in which, upon contacting a sample having formaldehyde fixed biomolecules (with the adduct reversal agent at a concentration in a range of from 3 mM to 7 mM), for 1 hour at 23° C., at pH 7, 90% or less (e.g., 85% or less, 80% or less, 75% or less, or 70% or less) of hemiaminals remain compared to the amount of hemiaminals prior to contact (see FIG. 11A). In some cases, a subject adduct reversal agent is one in which, upon contacting a sample having formaldehyde fixed biomolecules (with the adduct reversal agent at a concentration in a range of from 3 mM to 7 mM) , for 2 hours at 23° C., at pH 7, 90% or less (e.g., 85% or less, 80% or less, 75% or less, 70% or less, 65% or less, or 60% or less) of hemiaminals remain compared to the amount of hemiaminals prior to contact (see FIG. 11A).

In some cases, a subject adduct reversal agent is one in which, upon contacting a sample having formaldehyde fixed biomolecules (with the adduct reversal agent at a concentration in a range of from 3 mM to 7 mM) , for 1 hour at 23° C., at pH 7, 98% or less (e.g., 96% or less, 94% or less, 92% or less, or 90% or less) of aminals remain compared to the amount of aminals prior to contact (see FIG. 11B). In some cases, a subject adduct reversal agent is one in which, upon contacting a sample having formaldehyde fixed biomolecules (with the adduct reversal agent at a concentration in a range of from 3 mM to 7 mM) , for 2 hours at 23° C., at pH 7, 98% or less (e.g., 96% or less, 94% or less, 92% or less, 90% or less, 88% or less, 86% or less, etc.) of aminals remain compared to the amount of aminals prior to contact (see FIG. 11 B).

A subject adduct reversal agent can be used at any convenient concentration. For example, in some cases, adduct reversal agent is used at a concentration (e.g., a final concentration) in a range of from 0.2 mM to 500 mM (e.g., 0.5 mM to 400 mM, 0.5 mM to 300 mM, 0.5 mM to 200 mM, 0.5 mM to 100 mM, 0.5 mM to 50 mM, 0.5 mM to 40 mM, 0.5 mM to 30 mM, 0.5 mM to 25 mM, or 0.5 mM to 20 mM, e.g., at 1 mM, at 5 mM, at 10 mM, at 15 mM, at 20 mM, etc.). In some cases, a subject adduct reversal agent is used at a concentration (e.g., final concentration) in a range of from 0.01 mM to 1000 mM (e.g., from 0.01 mM to 750 mM, from 0.01 mM to 500 mM, from 0.01 mM to 300 mM, from 0.01 mM to 200 mM, from 0.01 mM to 100 mM, from 0.01 mM to 50 mM, from 0.1 mM to 1000 mM, from 0.1 mM to 750 mM, from 0.1 mM to 500 mM, from 0.1 mM to 300 mM, from 0.1 mM to 200 mM, from 0.1 mM to 100 mM, from 0.1 mM to 50 mM, from 0.5 mM to 1000 mM, from 0.5 mM to 750 mM, from 0.5 mM to 500 mM, from 0.5 mM to 300 mM, from 0.5 mM to 200 mM, from 0.5 mM to 100 mM, from 0.5 mM to 50 mM, from 1 mM to 1000 mM, from 1 mM to 750 mM, from 1 mM to 500 mM, from 1 mM to 300 mM, from 1 mM to 200 mM, from 1 mM to 100 mM, or from 1 mM to 50 mM), As will be appreciated by one of ordinary skill in the art, “used at a concentration” (or “final concentration”) refers to the concentration of the adduct reversal agent in the reaction mixture (sometimes referred to as the final reaction mixture).

TABLE 1
Examples of subject adduct reversal agents. The methods of synthesis of all
of the compounds of Table 1 are known to one of ordinary skill in the art. For example, for
synthesis of Compound 4, see Crisalli et. al., Org Lett. 2013 Apr. 5; 15(7): 1646-9, which is
hereby incorporated by reference in its entirety).

| Compound # | | |
|---|---|---|
| (other name) | Structure | IUPAC |
| Compound 1 | 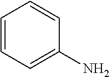 | Aniline |
| Compound 2 | 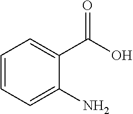 | 2-aminobenzoic acid |
| Compound 3 | 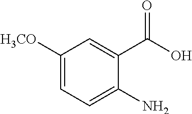 [SMILES: COc1ccc(N)c(C(=O)O)c1] | 2-amino-5-methoxybenzoic acid |
| Compound 4 | 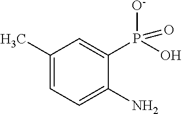 | (2-amino-5-methylphenyl)phosphonic acid |
| Compound 5 | 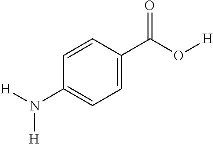 | 4-aminobenzoic acid |
| Compound 6 | 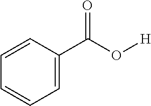 | benzoic acid |
| Compound 7 | 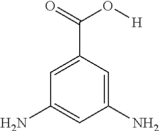 [SMILES: Nc1cc(N)cc(C(=O)O)c1] | 3,5-diaminobenzoic acid |
| Compound 8 | 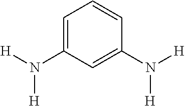 | benzene-1,3-diamine |
| Compound 9 | 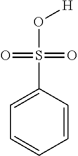 | benzenesulfonic acid |
| Compound 10 | 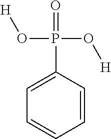 | phenylphosphonic acid |
| Compound 11 | 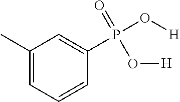 | (3-methylphenyl)phosphonic acid |
| Compound 12 | 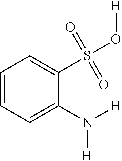 [SMILES: Nc1ccccc1S(=O)(=O)O] | 2-aminobenzene-1-sulfonic acid |
| Compound 13 | 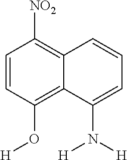 [SMILES: Nc1cccc2c([N+](=O)[O-])ccc(O)c12] | 8-amino-4-nitronaphthalen-1-ol |
| Compound 14 | 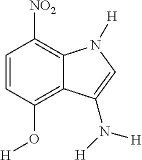 [SMILES: Nc1c[nH]c2c([N+](=O)[O-])ccc(O)c12] | 3-amino-7-nitro-1H-indol-4-ol |
| Compound 15 | 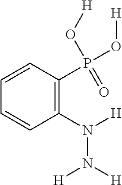 [SMILES: NNc1ccccc1P(=O)(O)O] | (2-hydrazinylphenyl)phosphonic acid |
| Compound 16 | 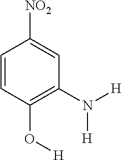 [SMILES: Nc1cc([N+](=O)[O-])ccc1O] | 2-amino-4-nitrophenol |
| Compound 17 | 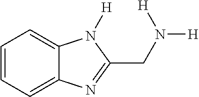 [SMILES: NCc1nc2ccccc2[nH]1] | 1H-1,3-benzodiazol-2-ylmethanamine |
| Compound 18 | 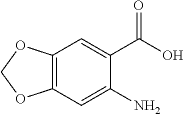 [SMILES: Nc1cc2c(cc1C(=O)O)OCO2] | 6-amino-2H-1,3-benzodioxole-5-carboxylic acid |
| Compound 19 | 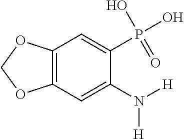 [SMILES: Nc1cc2c(cc1P(=O)(O)O)OCO2] | (6-amino-2H-1,3-benzodioxol-5-yl) phosphonic acid |
| Compound 20 | 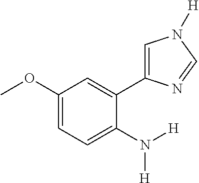 | 2-(1H-imidazol-4-yl)-4-methoxyaniline |

### Compositions and Kits

Also provided are compositions and kits for use in the subject methods. Examples of a subject composition includes: a composition having two or more of Compounds 1-20 of Table 1; and a composition having two or more of Compounds 1-12 of Table 1. In some cases, a subject composition includes: (a) a subject adduct reversal agent (e.g., Compound 4), and (b) a protease (e.g., trypsin, chymotrypsin, proteinase K, papain, pepsin, pronase, endoproteinase Lys-C, endoproteinase glu-C, and the like) or protease buffer (a buffer suitable for protease activity, i.e., a protease-compatible buffer). In some cases, a subject composition includes: (a) a subject adduct reversal agent (e.g., Compound 4), and (b) a protease (e.g., trypsin, chymotrypsin, proteinase K, papain, pepsin, pronase, endoproteinase Lys-C, endoproteinase glu-C, and the like). In some cases, a subject composition includes: (a) a subject adduct reversal agent (e.g., Compound 4), and (b) a protease buffer (a buffer suitable for protease activity, i.e., a protease-compatible buffer). In some cases, the buffer is a proteinase K compatible buffer. In some cases, a subject composition includes: (a) a subject adduct reversal agent (e.g., Compound 4), and (b) a chaotropic agent (e.g. guanidinium thiocyanate).

The components (e.g., an adduct reversal agent and a protease, an adduct reversal agent and a chaotropic agent, etc.) of a subject composition can be present as a mixture or can be separate entities. In some cases, the components (e.g., an adduct reversal agent and a protease, an adduct reversal agent and a chaotropic agent, etc.) are a lyophilized mixture. In some cases, the components (e.g., an adduct reversal agent and a protease) are a liquid mixture.

A subject kit can include any combination of components and compositions for performing the subject methods. In some embodiments, a kit can include one or more of the following: two or more of Compounds 1-20 of Table 1; two or more of Compounds 1-12 of Table 1; a composition having two or more of Compounds 1-20 of Table 1; and a composition having two or more of Compounds 1-12 of Table 1. In some cases, a subject kit includes: (a) a subject adduct reversal agent (e.g., Compound 4), and (b) a protease (e.g., trypsin, chymotrypsin, proteinase K, papain, pepsin, pronase, endoproteinase Lys-C, endoproteinase glu-C, and the like) or protease buffer (a buffer suitable for protease activity, i.e., a protease-compatible buffer). In some cases, a subject kit includes: (a) a subject adduct reversal agent (e.g., Compound 4), and (b) a protease (e.g., trypsin, chymotrypsin, proteinase K, papain, pepsin, pronase, endoproteinase Lys-C, endoproteinase glu-C, and the like). In some cases, a subject kit includes: (a) a subject adduct reversal agent (e.g., Compound 4), and (b) a protease buffer (a buffer suitable for protease activity, i.e., a protease-compatible buffer). In some cases, the buffer is a proteinase K compatible buffer. In some cases, a subject kit includes: (a) a subject adduct reversal agent (e.g., Compound 4), and (b) a chaotropic agent (e.g. guanidinium thiocyanate).The components of a kit can be in the same or separate containers, in any combination.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

In some cases, an adduct reversal agent (e.g, at least one adduct reversal agent) of a composition or kit is a compound having an aromatic ring and an amine group (e.g., a primary amine, a secondary amine, an aromatic amine). In some cases, an adduct reversal agent (e.g, at least one adduct reversal agent) of a composition or kit is selected from: Compounds 1-5, 7-8, and 12-20 of Table 1.

In some cases, an adduct reversal agent (e.g, at least one adduct reversal agent) of a composition or kit is a compound having an aromatic ring and a proton-donating group (e.g., a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, a boronic acid group, and the like). In some cases, an adduct reversal agent (e.g, at least one adduct reversal agent) of a composition or kit is selected from: Compounds 2-7, and 9-19 of Table 1.

In some cases, an adduct reversal agent (e.g, at least one adduct reversal agent) of a composition or kit is a compound having an aromatic ring, an amine group (e.g., a primary amine, a secondary amine, an aromatic amine), and a proton-donating group (e.g., a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, a boronic acid group, and the like). In some cases, an adduct reversal agent (e.g, at least one adduct reversal agent) of a composition or kit is selected from: Compounds 2-5, 7, 12-16, and 18-19 of Table 1.

In some cases, an adduct reversal agent (e.g, at least one adduct reversal agent) of a composition or kit is an aminobenzene having an ortho phosphonate group. In some cases, an adduct reversal agent (e.g, at least one adduct reversal agent) of a composition or kit is selected from: Compounds 4, 15, and 19 of Table 1.

In some cases, an adduct reversal agent (e.g, at least one adduct reversal agent) of a composition or kit is selected from: Compounds 1-12 of Table 1. In some cases, an adduct reversal agent (e.g, at least one adduct reversal agent) of a composition or kit is selected from the compounds of Table 1.

In some embodiments, a subject kit (e.g., a kit for reducing the number of adducts and/or crosslinks from biomolecules) includes: (a) an adduct reversal agent that includes an aromatic ring and at least one of: an amine and a proton-donating group; and (b) a buffer, where (a) and (b) are present in the same or separate containers. In some cases, at least one of (a) and (b) is lyophilized. In some cases, the buffer is an aqueous solution (e.g., an aqueous solution buffered to a pH in a range of from 4 to 8.5). As noted above, in some cases, the adduct reversal agent is a compound that includes an amine. In some cases, the amine is a primary amine or a secondary amine. In some cases, the amine is an aromatic amine. In some cases, the adduct reversal agent is a compound that includes a proton-donating group (e.g., a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, and/or a boronic acid group),In some cases, the pKa of the proton-donating group is in a range of from 4-8.5. In some cases, the adduct reversal agent is an aminobenzene having an ortho phosphonate group. In some cases, the adduct reversal agent is a compound selected from Table 1 (e.g., compound 4). In some cases, the kit includes a second adduct reversal agent that includes an aromatic ring and at least one of: an amine and a proton-donating group. In some cases, the kit includes a protease.

In some embodiments, a subject kit (e.g., a kit for reducing the number of adducts and/or crosslinks from biomolecules) includes (a) a first adduct reversal agent that includes an aromatic ring and at least one of: an amine and a proton-donating group; and (b) a second adduct reversal agent that includes an aromatic ring and at least one of: an amine and a proton-donating group, where (a) and (b) are present in the same or separate containers. In some cases, the first and second adduct reversal agents are each selected from the compounds of Table 1.

In some embodiments, a subject composition includes: (a) an adduct reversal agent that includes an aromatic ring and at least one of: an amine and a proton-donating group; and (b) a buffer. In some cases, the composition is lyophilized. In some cases, the composition is an aqueous solution. In some cases, the adduct reversal agent is a compound that includes an amine (e.g., a primary amine, a secondary amine, an aromatic amine). In some cases, the adduct reversal agent is a compound that includes a proton-donating group (e.g., a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, a boronic acid group). In some cases, the pKa of the proton-donating group is in a range of from 4-8.5. In some cases, the adduct reversal agent is an aminobenzene having an ortho phosphonate group. In some cases, the adduct reversal agent is a compound selected from Table 1 (e.g., compound 4). In some cases, the composition also includes a second adduct reversal agent that includes an aromatic ring and at least one of: an amine and a proton-donating group. In some cases, each of the adduct reversal agents are selected from the compounds listed in Table 1. In some cases, the composition further includes an aldehyde fixed biomolecule (e.g., one or more of: a nucleic acid, an amino acid, and a protein). In some cases, the composition includes a protease. In some cases, the composition includes a chaotropic agent. In some cases, the composition includes a protease and a chaotropic agent. In some cases, the composition is an aqueous solution buffered to a pH in a range of from 4 to 8.5.

In some embodiments, a subject composition includes: (a) a first adduct reversal agent that includes an aromatic ring and at least one of: an amine and a proton-donating group; and (b) a second adduct reversal agent that includes an aromatic ring and at least one of: an amine and a proton-donating group. In some cases, the first and second adduct reversal agents are each selected from the compounds listed in Table 1.

In some embodiments, a subject composition includes: (a) an adduct reversal agent dissolved in a buffered aqueous solution, wherein the adduct reversal agent includes an aromatic ring and at least one of: an amine and a proton-donating group; and (b) at least one aldehyde fixed biomolecule (e.g., one or more biomolecules selected from: a nucleic acid, an amino acid, and a protein).

### Utility

There are roughly 300 million tissue samples stored as formalin-fixed, paraffin-embedded (FFPE) specimens in the U.S. alone. They are indexed to disease, treatment, and outcomes, and if one could better mine the molecular data hidden in them, one could gain valuable molecular insights into diseases such as cancer and heart disease. It is currently often difficult to do this, as the formaldehyde used to crosslink tissue for stability also blocks the recovery of RNA and DNA sequence and protein antigens.

Aspects of the disclosure include compounds, methods, and kits for removing formaldehyde adducts from biomolecules (RNA, DNA, proteins) under mild conditions that preserve the structure and sequence of the biomolecules. This will have important applications in recovery of biomolecular data from preserved tissue (e.g. FFPE tissue specimens).

Methods are disclosed to reduce the number of chemical crosslinks and adducts induced by cross-linking fixatives, such as formaldehyde, so that biological molecules can be more reliably isolated from, identified in, or quantified in, biological samples (e.g., fixed tissue specimens). The biological molecules may be proteins or nucleic acids isolated for use in proteomic or genomic studies. The subject methods can also be used to identify antigens and enzymes in fixed tissue specimens that are subjected to immunohistochemical or enzyme histochemical studies. Reversal of fixation-induced adducts and/or crosslinking facilitates the determination of gene expression patterns (e.g., via in situ hybridization) and chromosomal alterations to be reliably determined in archived tissue samples (e.g., when performing proteomic and/or genomic studies related to development and progression of diseases such as cancer, neurological disorders, etc.).

The subject methods represent the first catalytic methods that greatly accelerate the removal of formaldehyde adducts to these biomolecules, and the methods can be carried out under very mild conditions that avoid damage to RNA (one of the least stable of biomolecules) and/or DNA. This will allow access to sequence and/or quantification where it was not possible before. For example, the subject methods will allow (i) RNA and DNA detection from samples, e.g., via PCR, via sequencing etc.; and (ii) improved “antigen retrieval” from samples, resulting in improved antibody-based antigen (e.g., protein) detection (e.g., enabling the acquisition of stronger signals by immunohistochemistry). The subject methods will also enhance signal where it was previously weak (e.g., due to reduced degradation of biomolecules in the sample compared to the degradation observed using current methods). Thus, the methods provide higher-yield retrieval of longer, less damaged RNAs, DNAs, and proteins from tissue specimens (resulting in stronger signals from PCR analysis; better, more complete DNA and RNA sequencing data from fewer numbers of reads; more reliable quantification in “dirty” samples comprising proteins and contaminants commonly encountered during nucleic acid purification (e.g. phenol, guanidine etc.) stronger signal detection when using in situ hybridization; stronger signal detection when detected proteins, e.g., via antibody-based methods, etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g.

amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nL); microliters (uL or μL); milliliters (mL); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); and the like.

Formaldehyde is often employed in fixation of tissue specimens, where it forms hemiaminal and aminal adducts with biomolecules, hindering the ability to retrieve molecular information from them. The common existing methods for removal of these adducts involve extended heating, which can cause extensive degradation and loss of nucleic acids, particularly RNA. The data presented here show that water-soluble bifunctional catalysts (anthranilates and phosphanilates) speed the reversal of formaldehyde adducts of mononucleotides by a substantial factor over standard buffers. The studies here with formaldehyde-treated RNA oligonucleotides show that the catalysts enhance the removal and uncrosslinking of adducts, restoring unmodified RNA at 37° C. even when extensively modified, and avoiding high temperatures that promote RNA degradation. The experiments here with formalin-fixed, paraffin-embedded cell samples show that the catalysis is compatible with common RNA extraction protocols and substantially enhances quantities of amplifiable RNA recovered. Such catalytic strategies can be used for reversing formaldehyde adducts in clinical specimens.

Experimental Methods

Materials and reagents. Adenosine monophosphate (AMP), deoxyadenosine monophosphate (dAMP), lithium perchlorate, and all organic catalysts (except Compound 4) were purchased from Sigma-Aldrich Co. Methanol-free 10% formaldehyde, EM grade was purchased from Polysciences, Inc. Solvents and reagents were purchased from Fisher Scientific, Aldrich or ACROS unless otherwise noted. 1.0 M Tris-HCl (pH 7.0) buffer was purchased from Invitrogen.

Synthesis of N⁶-hydroxymethyl-dAMP. Synthesis of the hemiaminal monoadduct was performed following a literature procedure (reference 21). The compound was purified by reverse phase HPLC using a TEAA buffer/acetonitrile gradient; see Supporting Information file for details. The product was characterized by high-resolution ESI-mass spectrometry as C₁₁H₁₇N₅O₇P (M+H).

Synthesis of Methylene-bis-adenosine-5′-monophosphate (dimer). 0.06 M solution of dAMP (in deionized water) and 0.3 M solution of 10% formaldehyde (methanol free) in 0.2 M sodium acetate buffer (pH 4.8) were mixed in equal volume. The mixture was stirred for 2-3 days at room temperature. The reaction mixture was then briefly stored by freezing at −20° C. After thawing, the crude product was precipitated from ice-cold 2% LiC10₄ in acetone and purified by reverse phase HPLC using a TEAA buffer/acetonitrile gradient. The product was characterized by HR-MALDI-MS as C₂₁H₂₉N₁₀O₁₄P₂ and further confirmed by ¹H NMR.

Compound 4. Phosphonate Compound 4 was synthesized via a new procedure in three steps from p-iodoaniline. All spectral properties matched those of the same compound synthesized by our previous route (reference 20).

Reverse crosslinking of monoadduct and dimer monitored by HPLC. Reactions were carried out on 1 mL scale. 100 μL of reaction mixture was collected at one-hour intervals and injected into the HPLC (same conditions as for the purification of monoadduct and dimer). The progress of the reaction (reverse cross linking) was monitored at 260 nm.

Oligoribonucleotide synthesis. An RNA oligomer containing a self-complementary region (5′-AAAAACGCGCGAAAAA-3′, 5181.31 Da) was designed and synthesized using standard 13-cyanoethyl phosphoramidite chemistry and 2′-O-TBDMS-protected ribonucleosides. Phosphoramidites were purchased from Glen Research. Deprotection and initial purification of the RNA were carried out using Glen-Pak RNA purification columns. The oligonucleotide was further purified using polyacrylamide gel electrophoresis. The RNA was analyzed by MALDI-MS.

RNA formaldehyde treatment. To 1 equivalent of RNA stock solution (650 μM or 325 μM) in a 200 μL microcentrifuge tube was added 2 equivalents of methanol-free 10% formaldehyde solution with 1 M sodium chloride; the preparation scale ranged from 3 to 60 μL. The RNA-formaldehyde mixture was incubated at room temperature for 24 hours, after which time the RNA was isolated by ethanol precipitation. The pellet was either redissolved immediately or stored at −80° C. until use (within 24 hours).

RNA analysis by MALDI-MS. After formaldehyde treatment and post-treatment as described above, 1 μL of 50 μM DNA standard (5′-TCGGATCGTGATAT-3′, 4293.86 Da) was added to each reaction. The samples were then desalted using C18 ZipTips (EMD Millipore) and eluted directly onto a 100-well plate, on which they were cospotted with 3-HPA matrix containing ammonium citrate. MALDI-TOF mass spectrometry analysis was carried out on an ABI Voyager-DE RP mass spectrometer in linear negative ion mode. Spectra were recorded from 500 to 12000 Da. All experiments were repeated five times, with 100-200 shots per spectrum and at least two spectra taken from each spot. In each case, the spectrum with the highest signal-to-background ratio was used for subsequent analysis.
The program Data Explorer was used to extract data. 5-point Gaussian smoothing was carried out, along with automatic baseline correction, calibration relative to the internal DNA standard and peak detection with a 1% intensity cutoff. Peak data was imported to Microsoft Excel for analysis. The amount of intact RNA was measured by taking the ratio of the RNA peak height at 5181.3 Da to the reference DNA peak height at 4293.86 Da. The recovery of RNA from adducts was calculated by comparing the peak height of the intact RNA (5181.3 Da) to the sum of the peak heights of intact RNA and adducts: peaks between 5181.3 Da and 5676 Da with at least 10% intensity. Finally, degradation was quantified by measuring ion counts for the total non-degraded RNA (intact RNA+adducts) relative to the reference DNA.

RNA recovery from FFPE specimens. RNA was extracted from a FFPE Raji cell pellet using either the spin-column-based AllPrep® DNA/RNA FFPE kit (Qiagen), according to the manufacter's protocol, or a phenol-chloroform-isoamyl alcohol (PCI) extraction procedure.²⁶ Compound 4 was incorporated into the AllPrep® protocol by addition of an aqueous solution of 3 at pH 7 to the RNA-containing lysate. The mixture was heated at the stated temperate and for the stated time, then isolation continued as described in the manufacturer's protocol. RNA was quantified using the Qubit® RNA HS Assay Kit (Life Technologies). 200 ng RNA was used to synthesize cDNA using the Invitrogen SuperScript® III First-Strand Synthesis System for RT-PCR (Life Technologies). qPCR was performed in 384-well plates using SYBR® Green dye. Full experimental details for preparation of the FFPE sample, RNA isolation, quantification, cDNA synthesis, and qPCR are provided below.

### Results and Discussion

### Preparation and Characterization of Model Formalin-Adducted Nucleotides

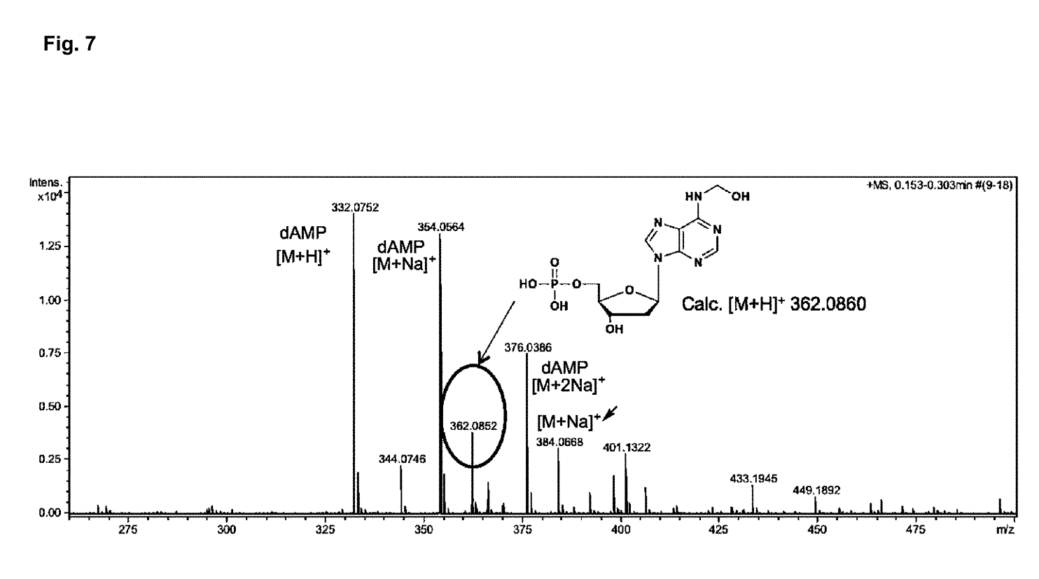

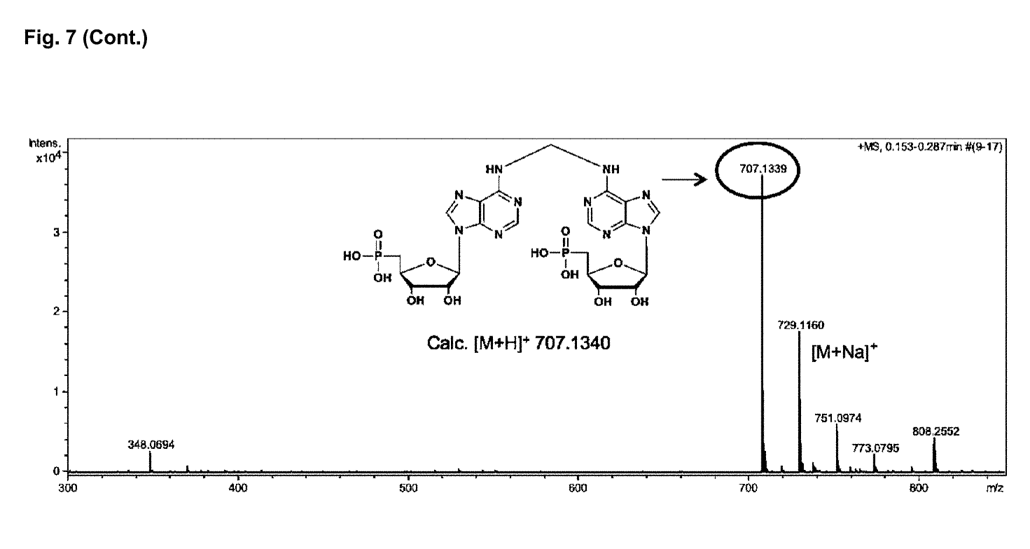

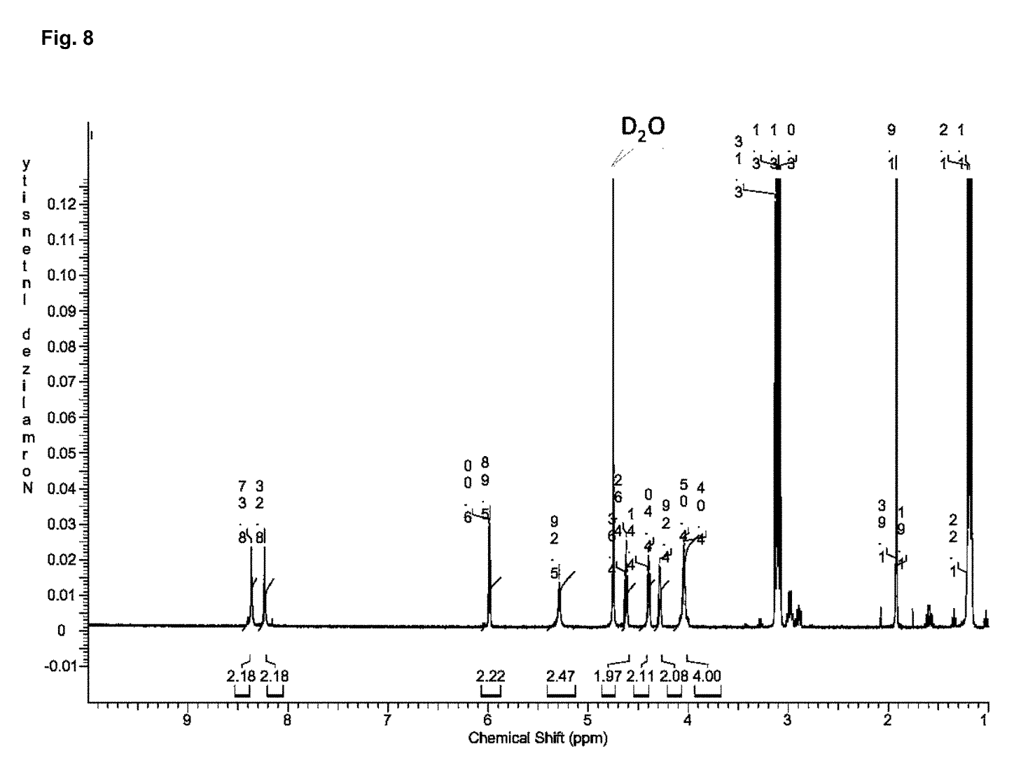

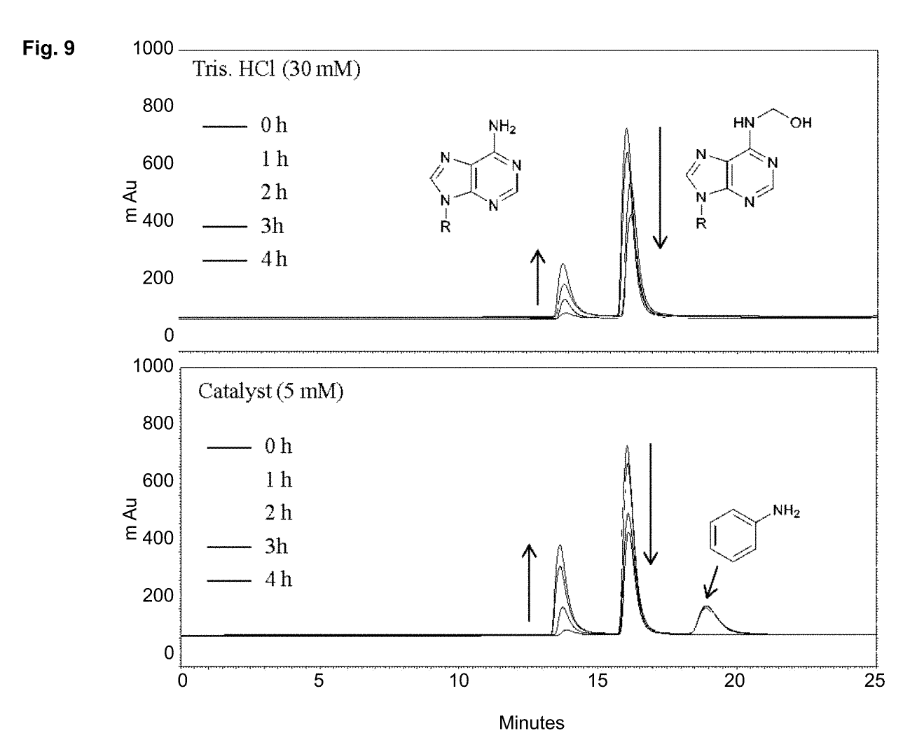

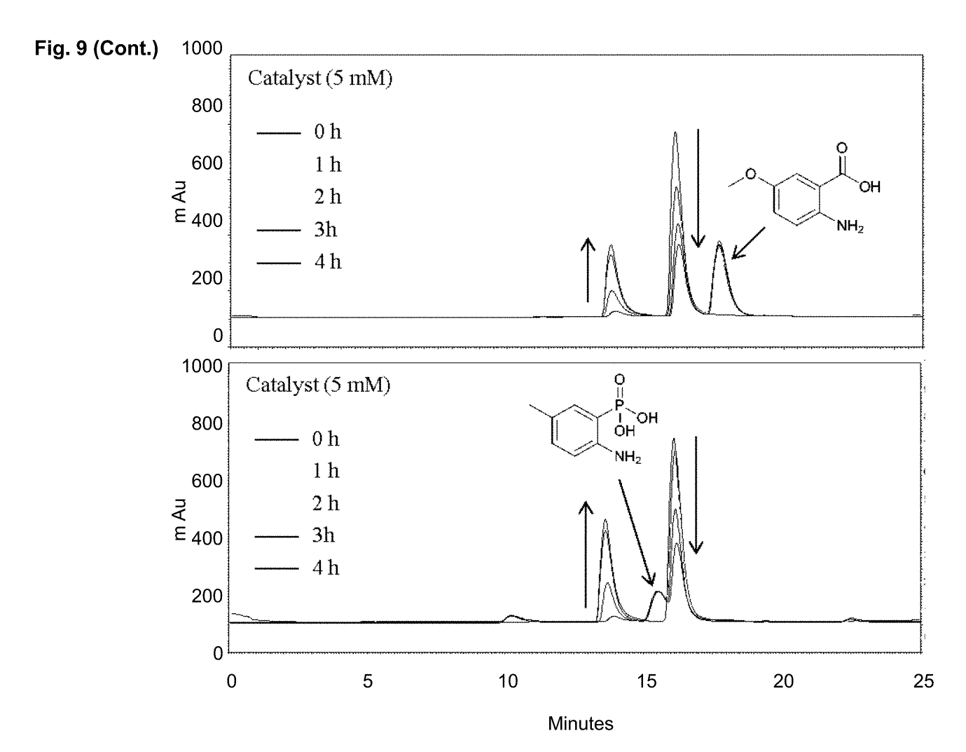

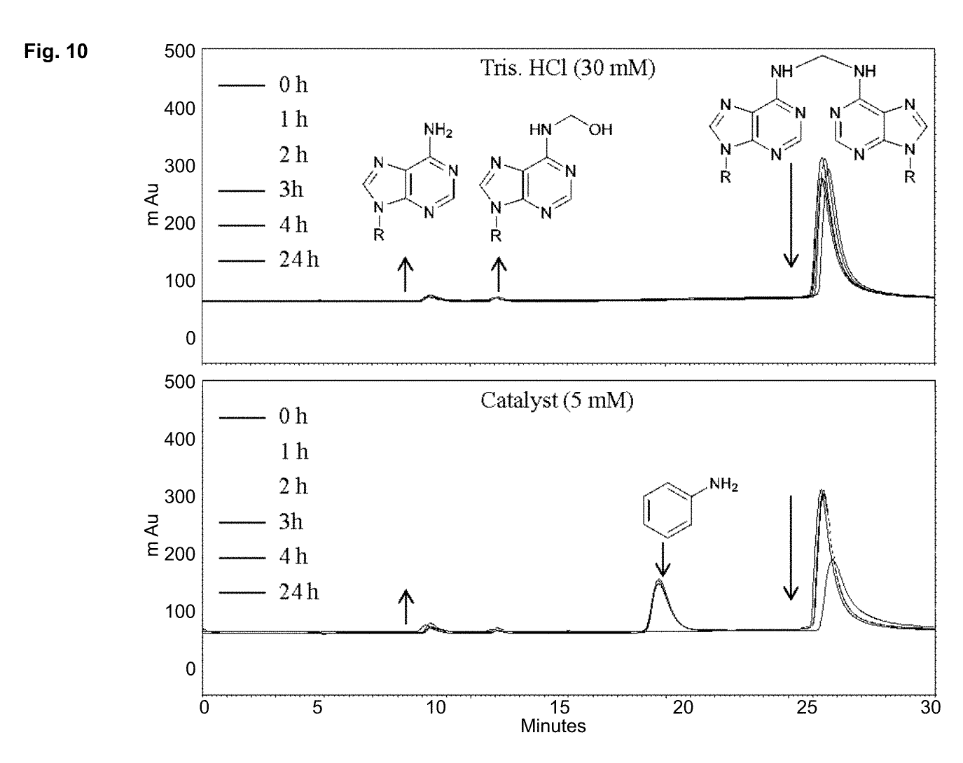

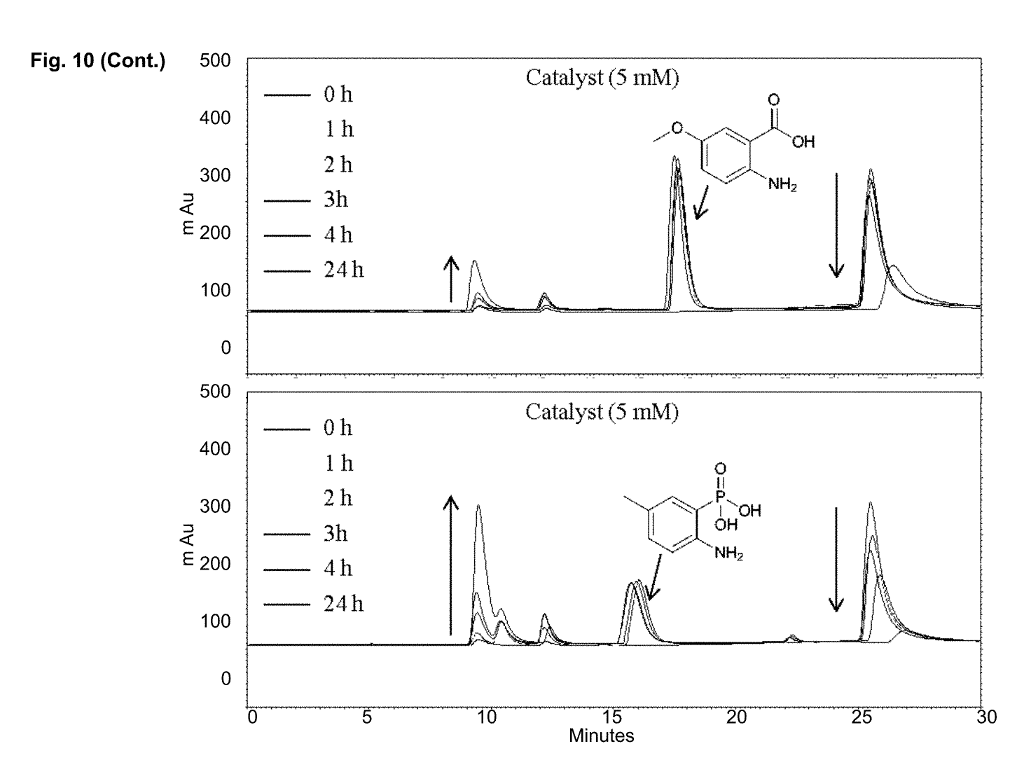

Water-soluble adducts of monomeric nucleotides AMP and dAMP were preprated that could serve as well-behaved, kinetically characterizable models of polymeric nucleic acids. The N6-hydroxymethyl monoadduct of dAMP²¹ was prepared in the presence of 10% formaldehyde (see details in Supporting Information). NMR and mass spectrometric analysis show that it exists primarily in the hemiaminal state (FIG. 1A-1C), presumably in equilibrium with the dehydrated imine form, which is not observed. Reverse-phase HPLC was used to resolve the monoadduct from unmodified dAMP (FIG. 7, FIG. 8, FIG. 9); in pH 7.0 buffer at room temperature, we find that this nucleotide formaldehyde adduct slowly reverts to unmodified dAMP, with a half-life of ca. 6 h (23° C.). For an aminal crosslink model, a previously unknown N6-dimer of AMP was prepared in buffer by extended incubation of AMP with 10% formaldehyde²². Experiments revealed that this aminal is much more stable than the hemiaminal model, showing little or no reversal to the uncrosslinked state in neutral buffer over at least a day at room temperature as monitored by HPLC (FIG. 10).

### Bifunctional Catalysts Promote Removal of Formaldehyde Adducts

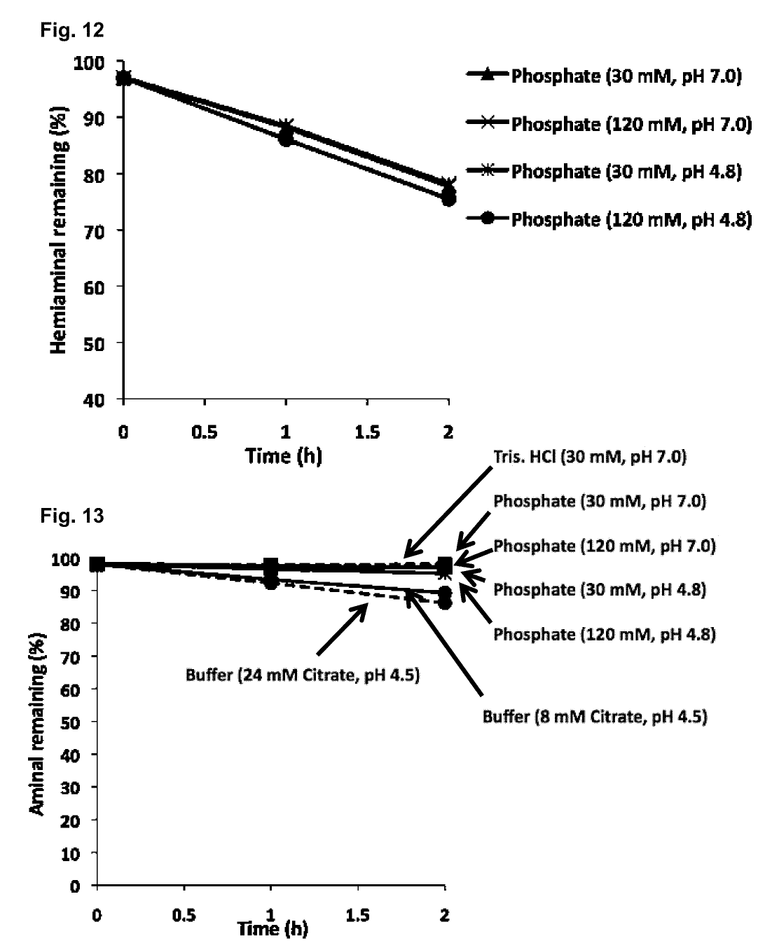

Having these model systems in hand, the effects of potential catalysts on the reversal of the adducts was next tested. A range of arylacids and amines was screened, including a number of bifunctional compounds containing both groups. Compounds were tested at 5 mM in 30 mM pH 7 Tris buffer, and yields of reversal were measured after 1 and 2 h at 23° C. (FIG. 11A-11B, FIG. 12). Although arylphosphonic acids and arylsulfonic acids were initially found to be active, they were removed from further consideration after it was found that their activity resulted from solution acidification after overwhelming the buffer. Of the remaining compounds, phosphanilate Compound 4 was found to promote substantial adduct reversal for both the hemiaminal and aminal models. Bifunctional anthranilate Compound 3 was also among the more active compounds. Notably, both compounds were previously identified as highly active catalysts for imine formation reactions¹⁹⁻²⁰. For formaldehyde adduct reversal, the effects of catalysts added at 5 mM were substantial; for example, the monoadduct was >50% reversed after 2 h with Compound 4, whereas in buffer alone only 11% reversal was seen. Although the aminal dimer was much more stable, showing ˜0.5% reversal in buffer at 2 h, the catalyst yielded ca. 14% reversal at this time point (23° C.).

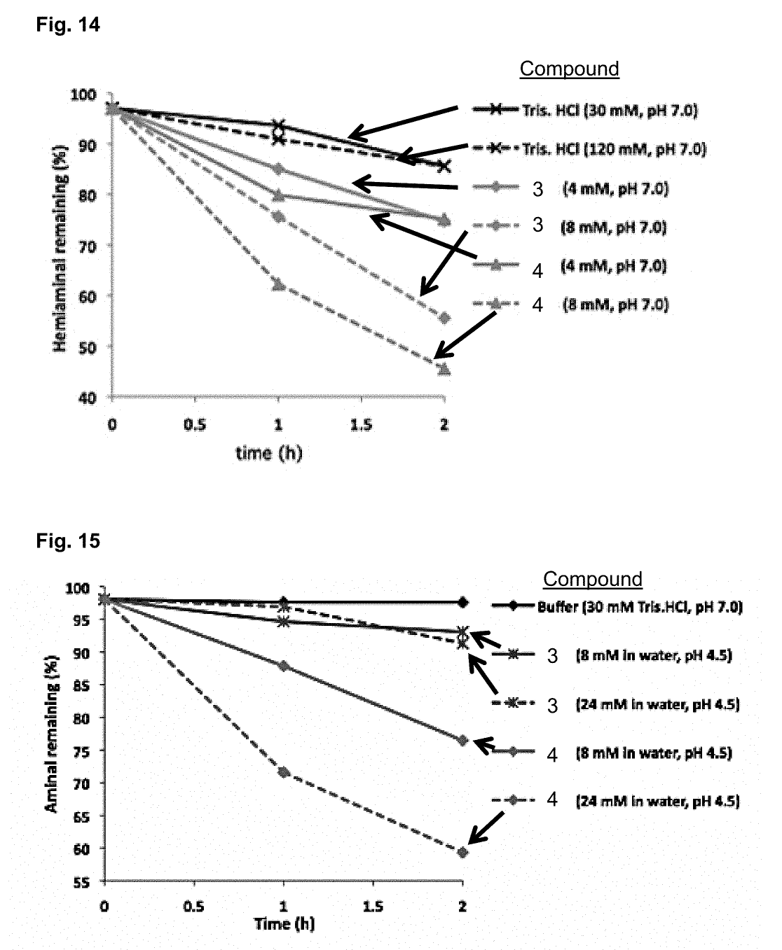

A more detailed analysis of the reactions catalyzed by bifunctional Compound 3 and Compound 4 was next carried out, comparing them to the classic transimination catalyst aniline (Compound 1) (reference 23). Testing both reactions at pH 4.5 and 7, Compound 4 was effective in reversal of the hemiaminal adduct at pH 7, while the catalyst was more effective at lower pH with the aminal dimer (most experiments were carried out at pH 4.5; FIG. 12, FIG. 13, FIG. 14, FIG. 15). Measurement of reaction yields at early time points allowed us to assess relative rates (FIG. 2A-2B). For the hemiaminal reversal reaction, we found that phosphanilate Compound 4 (10 mM) enhanced rate over buffer alone by 4-fold (pH 7.0, 23° C.), and by a factor of 3 over aniline. For the more stable aminal dimer, we found that Compound 4 accelerated the rate of dimer reversal by 37-fold over buffer alone and by 10-fold over aniline (pH 4.5, 23° C.) (FIG. 2A-2B). For both reactions, Compound 3 fell between Compound 4 and aniline in its ability to promote adduct reversal.

In order to gain more insight into the mechanism by which these catalysts were active, experiments were carried out that varied conditions and catalysts. First, by varying buffer pH the role of acid catalysis on the rate of adduct reversal was investigated. The hemiaminal reversal was relatively insensitive to pH, based on the observation that rates of reaction were little different in phosphate buffers at pH 7 or pH 4.5 (FIG. 12). In contrast, the aminal crosslinked dimer was affected by pH, with substantial rate accelerations occurring at lower pH in citrate buffer (FIG. 13). This suggests that aminal reversal is subject to specific acid catalysis, presumably by direct protonation of one of the nitrogens of the aminal²⁴. On the other hand, hemiaminal reversal does not appear to be subject to specific acid catalysis over this pH range, which is consistent with its lower basicity. Next catalyst concentrations were varied, using Compound 4 to test for evidence of general acid catalysis. The results showed that for both reactions, increasing concentrations of Compound 4 led to approximately proportionate rate enhancements (FIG. 14, FIG. 15). This establishes the presence of the catalyst at the transition state for these reactions, and is consistent with general acid catalysis as part of its mechanism of action. Notably, the buffers did not show concentration dependence in these two reactions, and thus are likely not interacting with the substrates at the transition states.

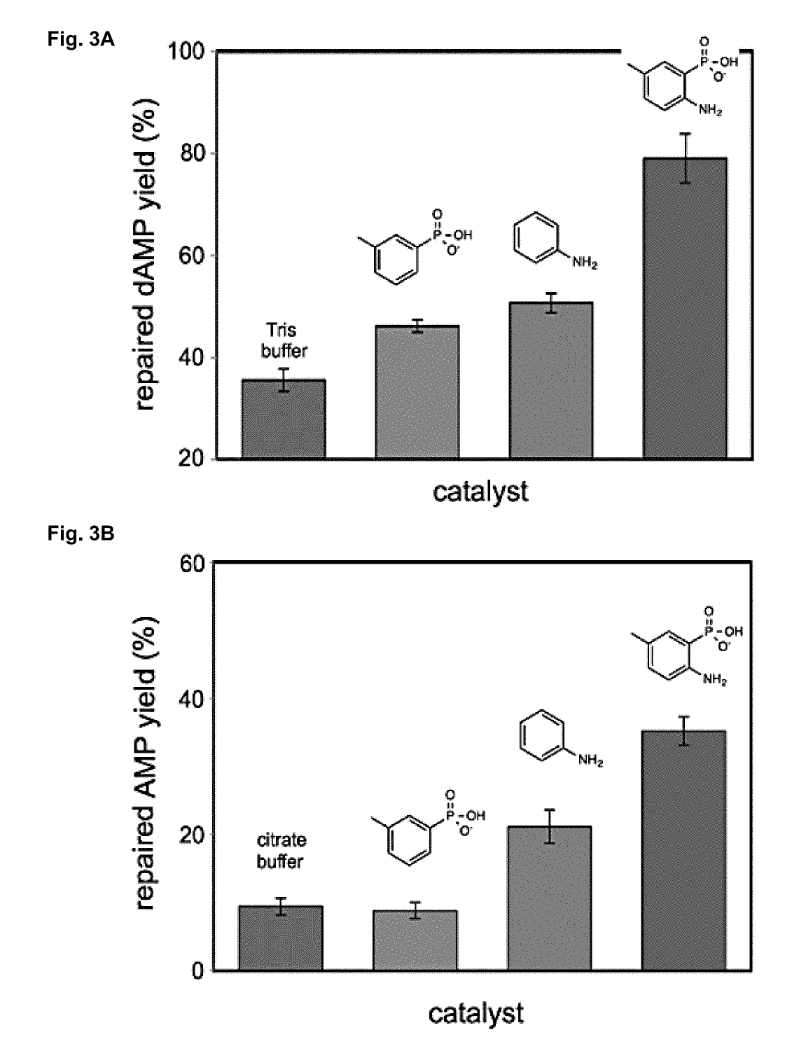

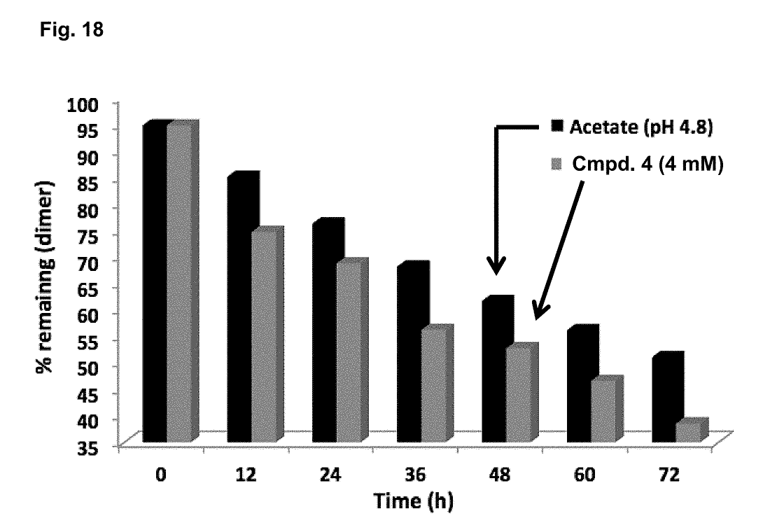

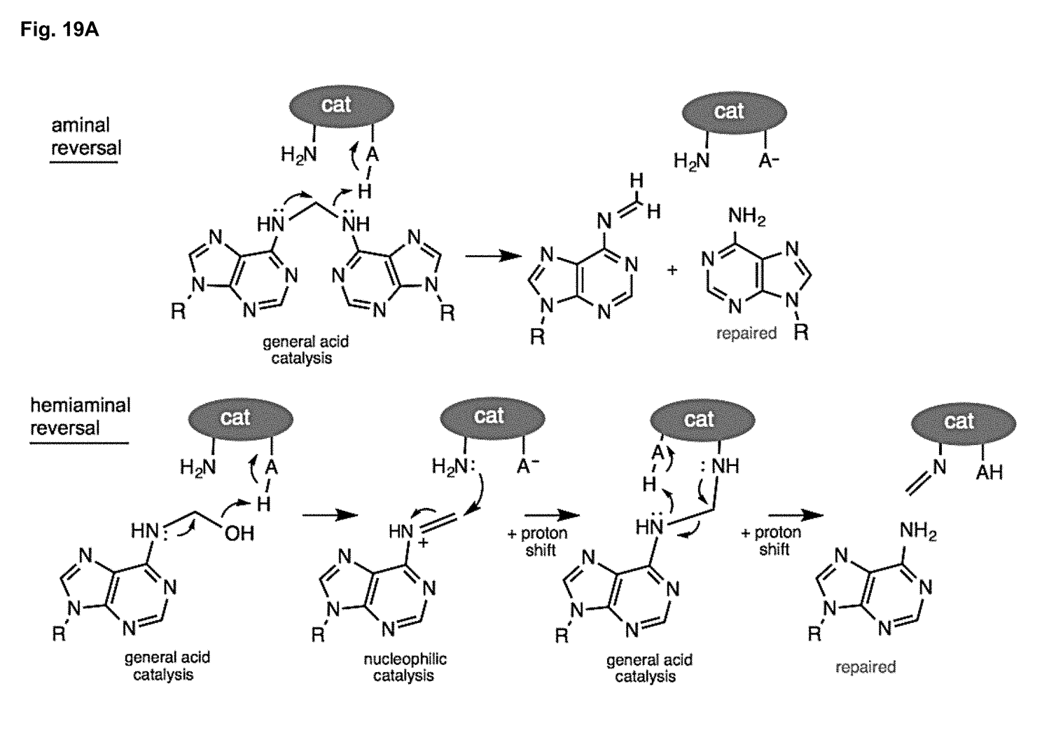

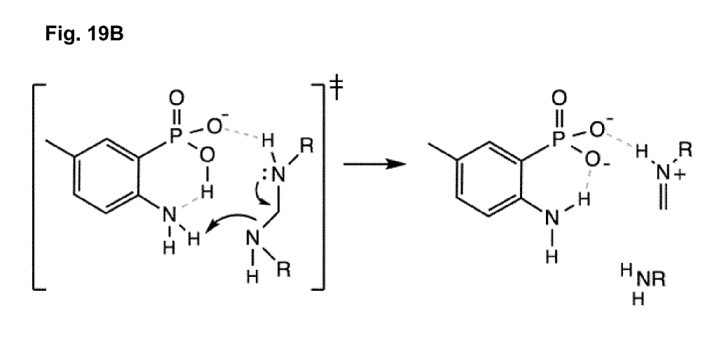

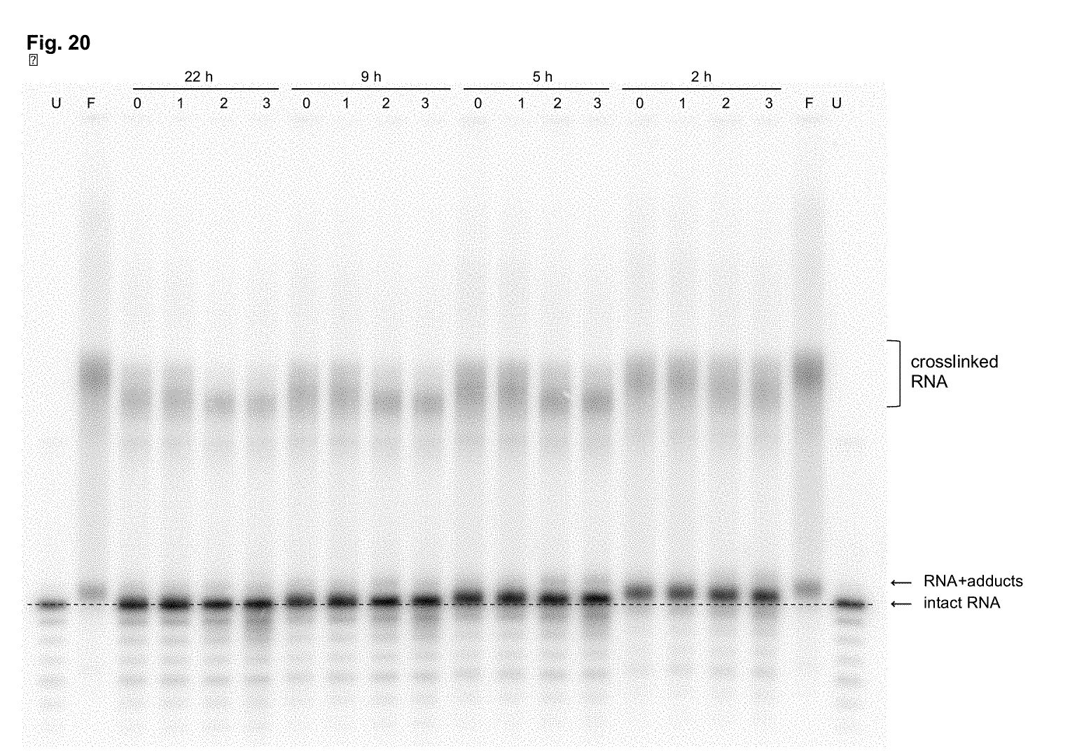

Since Compound 4 was the most efficient promoter of formaldehyde adduct reversal among compounds tested, the role of its structure was investigated by comparing it to analogs having amine or phosphonate functional groups omitted. The results showed (FIG. 3A-3B, FIG. 16, FIG. 17) that both of these functional groups are necessary to achieve most effective catalysis, as controls with amine or phosphonate groups alone yielded slower reaction. Whether bifunctional Compound 4 could foster true turnover catalysis was tested in an experiment using high concentration of aminal dimer, in molar excess over catalyst (FIG. 18); the data showed a catalyst advantage relative to buffer over at least three apparent turnovers. However, some product inhibition may occur at high formaldehyde concentrations, since an adduct between Compound 4 and formaldehyde was observable by NMR (data not shown). The issue of turnover is likely moot in application with formalin-fixed tissues, where catalyst is expected to be in large excess over substrate. Overall, the data show that bifunctional catalysts are most effective in these reactions, and that general acid catalysis in promoting bond breakage is likely to play a role in their mechanism. It is possible that nucleophilic catalysis may also be involved, since transimination likely proceeds via this mechanism (reference 23). A hypothetical mechanism that explains roles for both the acid and amine functional groups is proposed in FIG. 19A-19B, although further mechanistic studies are needed to confirm this hypothesis.

### An RNA Oligonucleotide is Extensively Modified by Formaldehyde

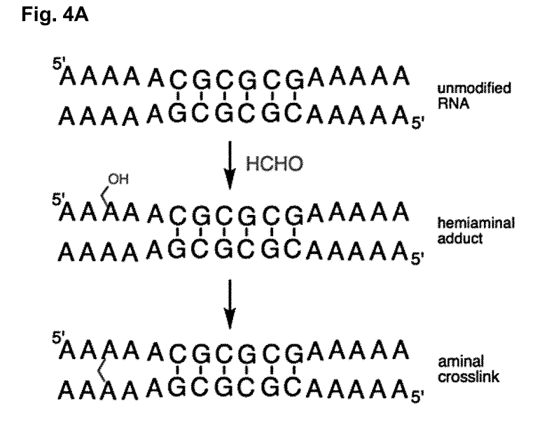

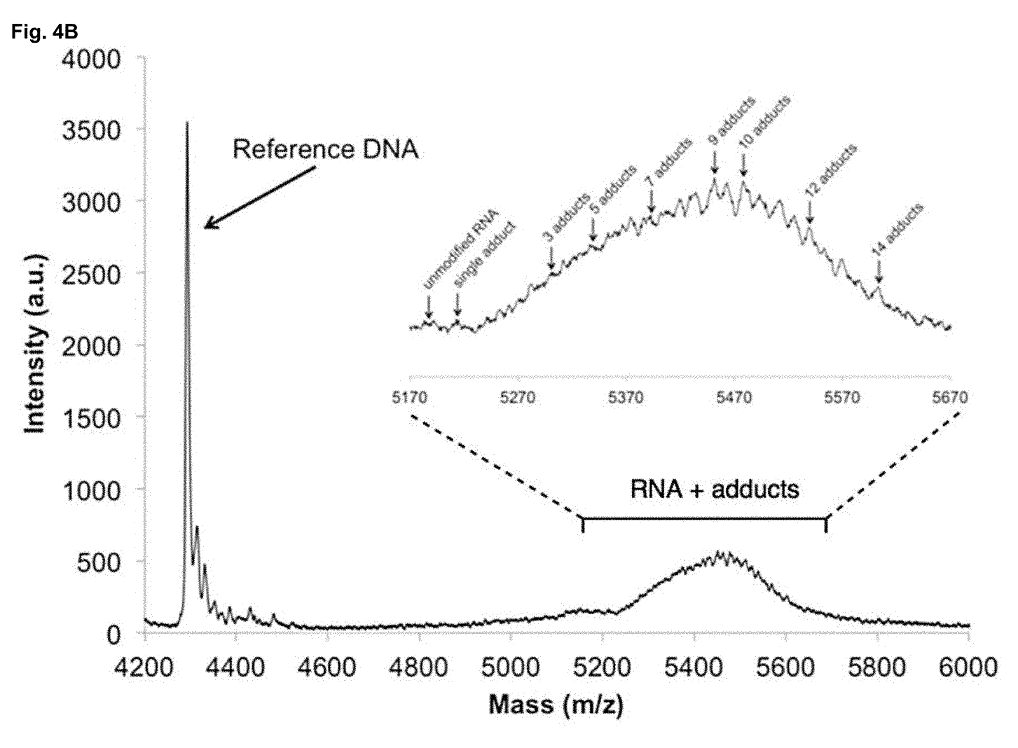

Next a full RNA strand model was developed to assess formaldehyde adducts in the biopolymer. This was done to investigate (a) to what extent formaldehyde adducts are formed on a native biopolymer as opposed to a mononucleotide; (b) to what degree standard heating-in-buffer protocols affect biopolymer stability and adduct removal, and (c) whether the observed catalysis of the reversal of formaldehyde adducts from a mononucleotide might extend to polymeric RNA. To this end, a 16 mer RNA strand having a central self-complementary sequence flanked by 5 mer (A)₅ ends was designed. This is expected to form a short duplex (FIG. 4A) with several overhanging adenosines to promote adduct and crosslink formation in the unpaired bases. To analyze adduct numbers and types qualitatively and quantitatively, we used denaturing polyacrylamide gel electrophoresis and MALDI-mass spectrometry. To generate adducts, the RNA was treated with 10% formaldehyde for 24 h at 23° C., precipitated, then redissolved in buffer for analysis of reversal.

Analytical data with this formaldehyde-treated RNA showed clear evidence of extensive adduct formation. PAGE analysis revealed evidence of a high degree of crosslinking to form dimers, and also showed shifting of the monomer RNA band, consistent with the base adducts causing slowing of mobility during electrophoresis (FIG. 20). These observations suggested that little unmodified RNA remains under these treatment conditions. Mass spectrometric analysis confirmed this, revealing no detectable unmodified RNA after formaldehyde treatment, while untreated RNA gave a clear parent ion peak at 5181 Da (FIG. 4A-4B). The observed masses after formaldehyde treatment corresponded to RNA containing ˜1-14 adducts, with a median of ca. 9 adducts. The monomer RNA contains 16 exocyclic amine groups on adenine, cytosine and guanine, and thus the results show that a large fraction of these groups on each RNA molecule carry an adduct. It is also possible that more than one adduct can form on a given exocyclic amine. Closer inspection of the mass spectrum of products around the monomeric RNA mass range (5000-6000 daltons) shows peaks consistent with increasing numbers of adducts primarily in hemiaminal form, adding 30 daltons of mass successively (see FIG. 4B).

Catalyst-Enhanced Removal of Formaldehyde from an RNA Oligonucleotide at Low Temperature

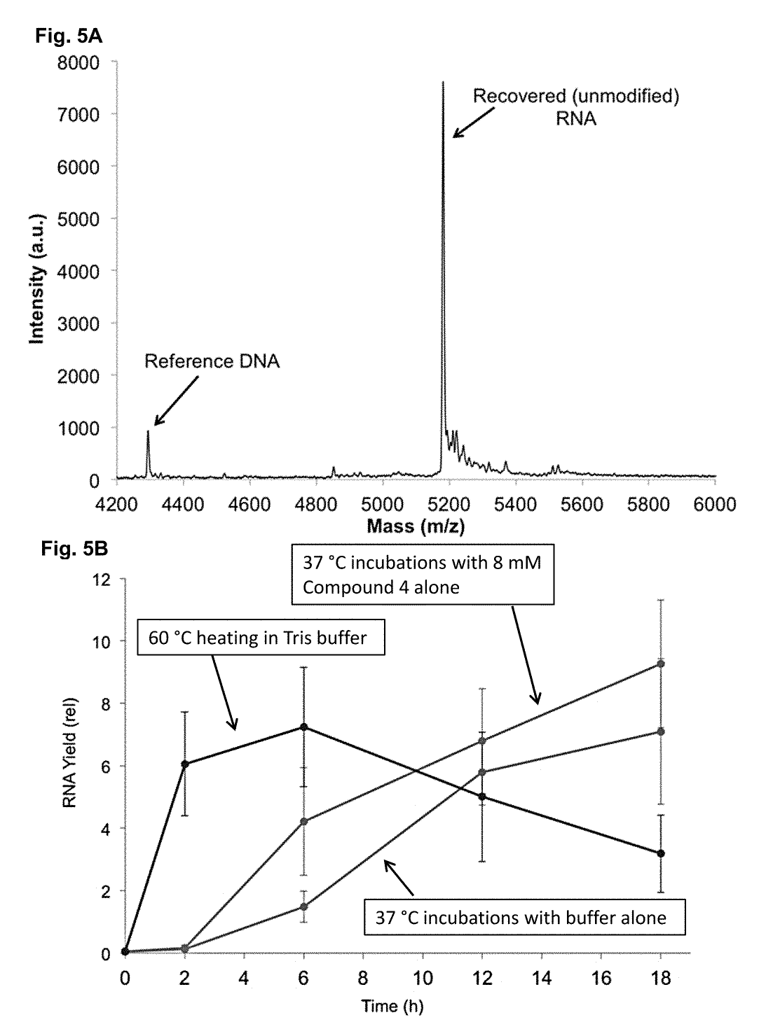

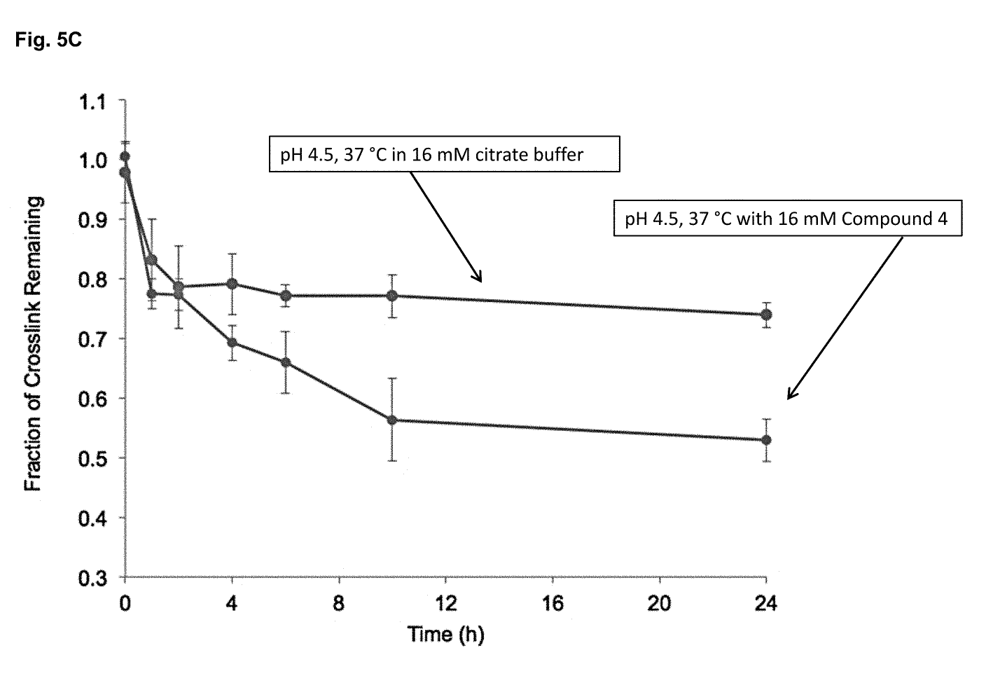

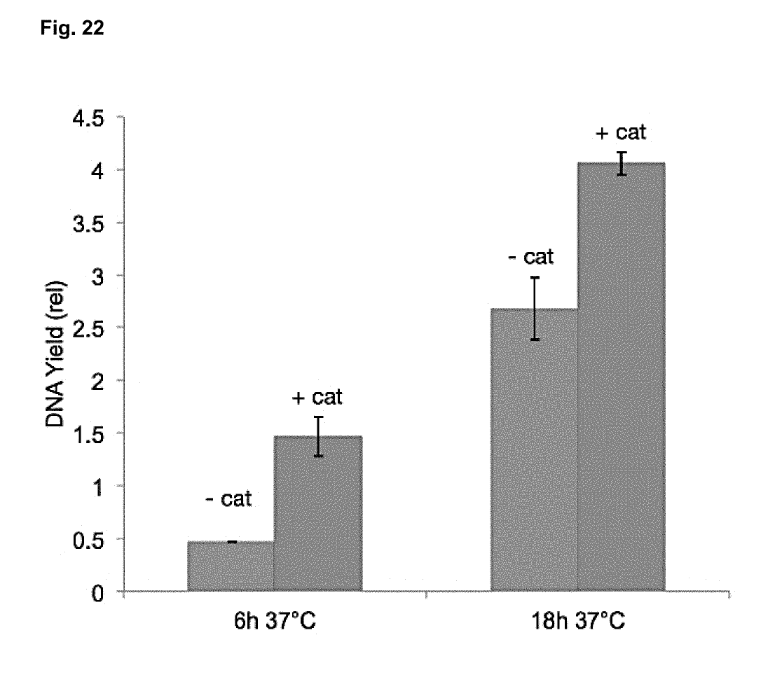

Using this extensively formaldehyde-modified RNA, we proceeded to test the effect of catalysts and conditions on loss of adducts and on recovery of intact RNA. Simple incubation in the presence of 8 mM catalysts at pH 7 (37° C.) showed relatively rapid apparent loss of adducts as judged by gel electrophoresis, with more efficient loss of crosslinked and shifted bands in the presence of catalysts as opposed to Tris buffer alone (FIG. 20). Analysis by mass spectrometry of adduct RNAs having mass near that of the 16 mer confirmed this; by analyzing counts of mass peaks relative to a DNA control spiked into the sample we were able to quantitatively measure amounts of hemiaminal adducts and intact RNA. Incubation at 37° C. with the catalyst over 18 h revealed loss of adducts and recovery of intact unmodified RNA as a major peak by mass spectrometry (FIG. 5A). The quantitative data showed that Compound 4 effectively enhanced the rate of adduct loss, increasing the ratio of intact RNA generated relative to Tris buffer alone at 37° C. Heating at a literature-standard 60° C. in buffer increased the rate of loss of formaldehyde adducts, but importantly, it also caused rapid degradation of the RNA, with >40% degraded after 12 h (FIG. 5B, FIG. 20). Thus the data suggest strongly that heating is to be avoided in RNA recovery²⁵, and that milder incubation at 37° C. in the presence of catalysts is effective at recovering a high yield of unmodified RNA from extensive hemiaminal adducts after 12-18 h incubation, even given an extensive degree of modification. Similar catalyst incubation with a formalin-treated self-complementary DNA duplex also revealed enhanced loss of formaldehyde adducts from this double-stranded biopolymer relative to buffer treatment alone (FIG. 22).

Next we explicitly tested catalyst effect on aminal-crosslinked RNAs, by quantifying dimer RNA bands and their conversion to monomer RNAs via denaturing gel electrophoresis analyzed by phosphorimaging. The data show that Compound 4 enhances the reversal of crosslinks between two 16 mer RNA strands, yielding a consistently greater degree of uncrosslinking relative to buffer alone at the same pH (FIG. 5C).

Catalyst-Enhanced Recovery of RNA from Paraffin-Embedded Formalin-Fixed Cells

Cellular RNAs fold into highly varied structures and thus are expected to react with formaldehyde to yield a greater diversity of hemiaminal and aminal adducts than tested in the above model systems. Moreover, recovery of RNA from formalin-fixed tissues requires additional steps including paraffin removal and proteolysis. Thus we sought to test whether our organocatalytic approach would retain benefits in RNA recovery in the more complex cellular milieu, and whether the presence of catalyst might interfere with the common steps of proteolysis, extraction and isolation, and PCR amplification that are carried out with clinical FFPE specimens. Because primary FFPE specimens from patients are highly variable from sample to sample²⁵, we prepared reproducible FFPE specimens via a literature approach²⁶, using a cultured cell line (Raji cells²⁷), treating the cell pellet with formalin and embedding it in a paraffin block following standard procedures²⁶.

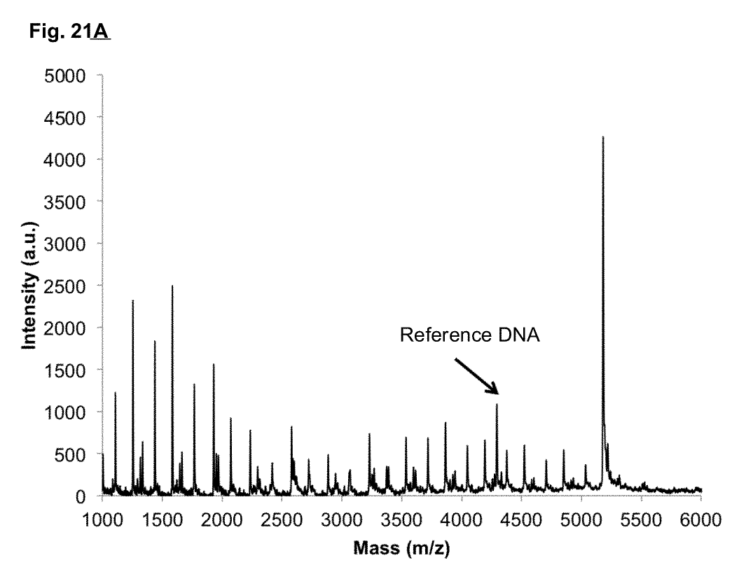

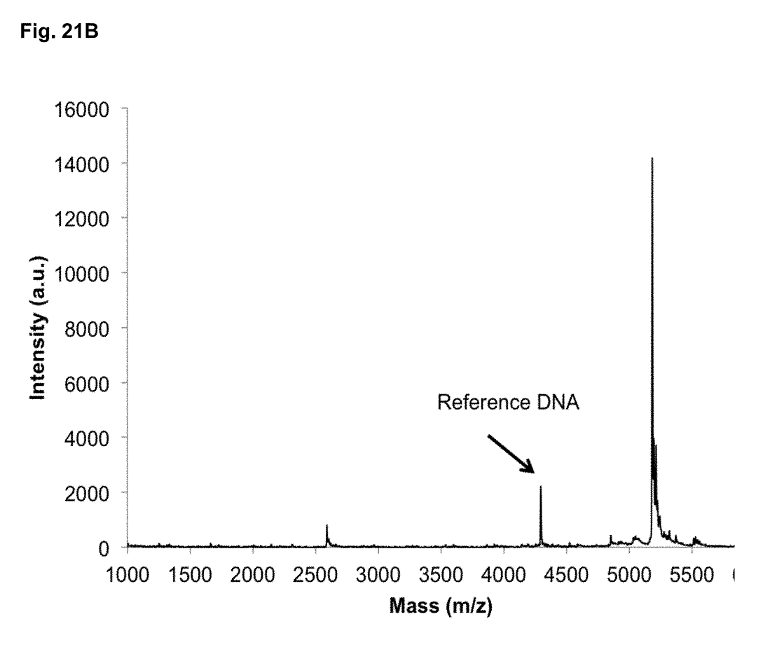

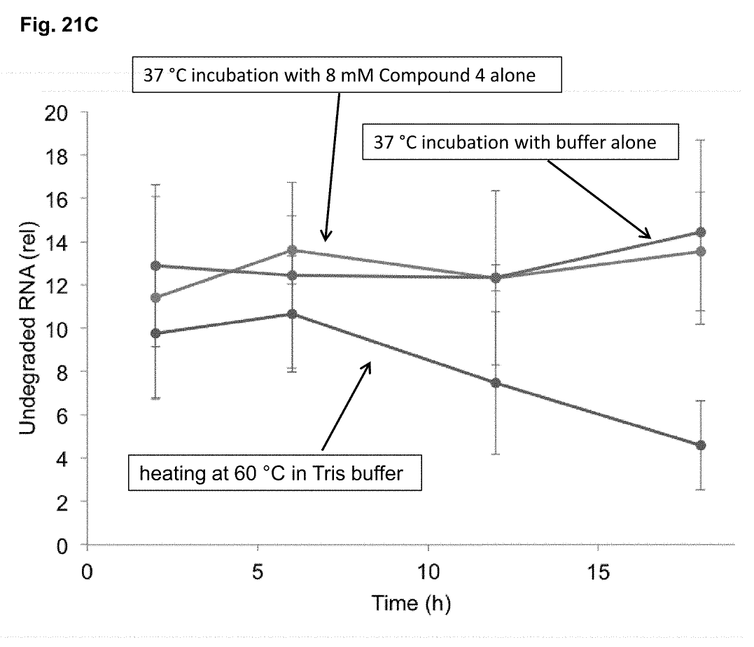

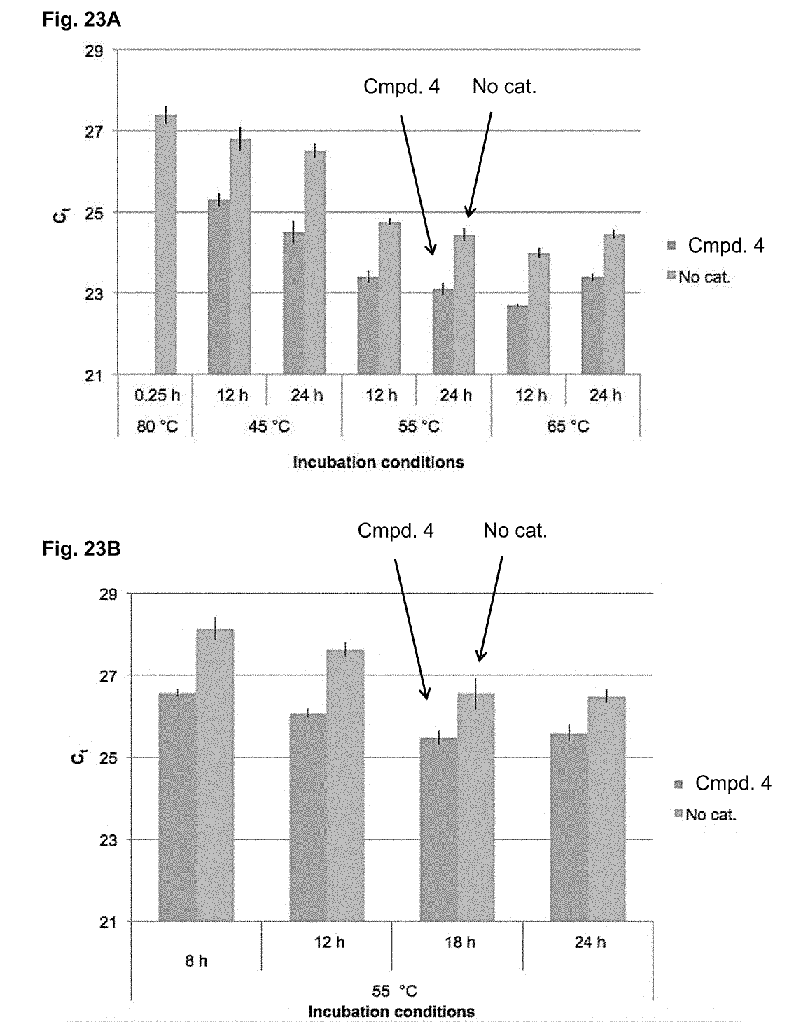

Formalin-fixed samples were then deparaffinized with xylenes and treated with proteinase K following standard procedures with a widely used commercial FFPE extraction kit. Compound 4 was then added (20 mM), and incubation times and temperatures varied to optimize RNA yield (FIG. 23A-23B), with an eye to avoiding high temperatures that rapidly degrade RNA (FIG. 21A-21C). RNA was then recovered by commercial silica-based spin columns from the kit; experiments showed that this method of RNA isolation (quantified by qRT-PCR) was not adversely affected by the presence of catalyst. Notably, the addition of catalyst increased yields at all temperatures and times tested, and the presence of catalyst increased amounts of detectable RNA when added to the commercial kit or to a highly cited incubation protocol²⁸. However, RNA recovery was yet higher when lower temperatures were employed, thus avoiding heat-induced RNA degradation by hydrolysis.

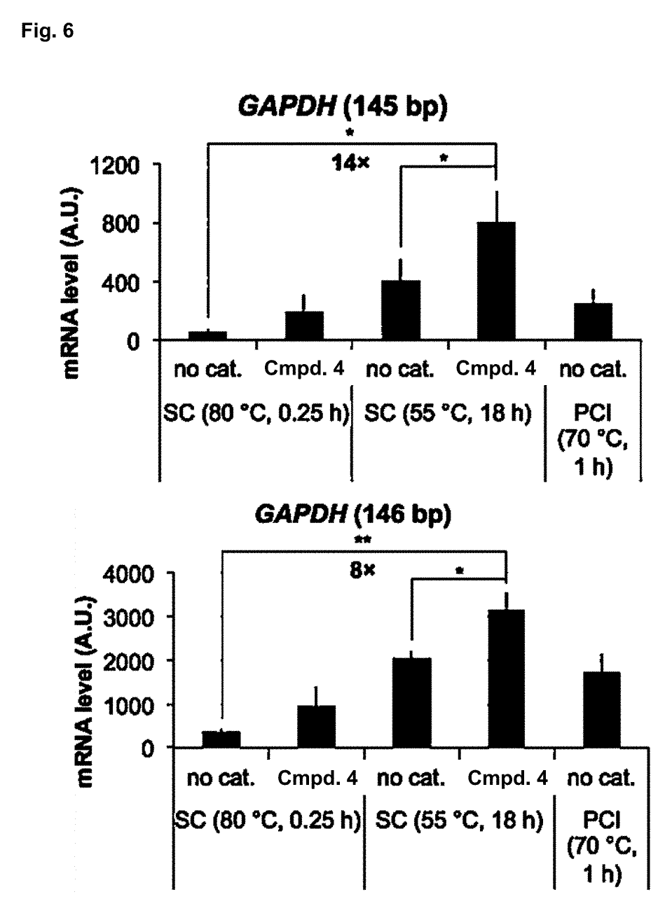

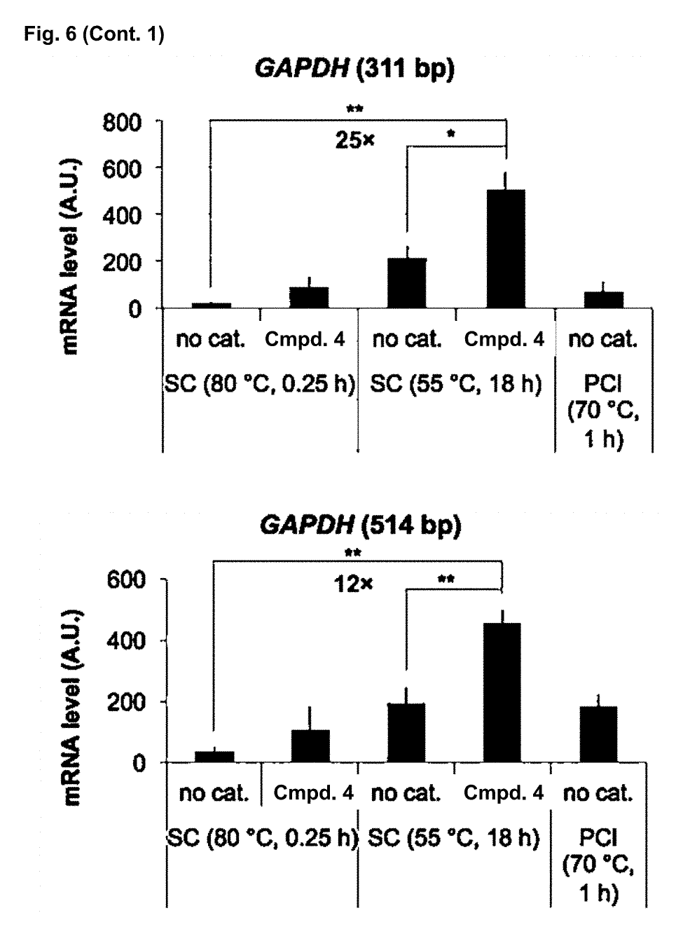

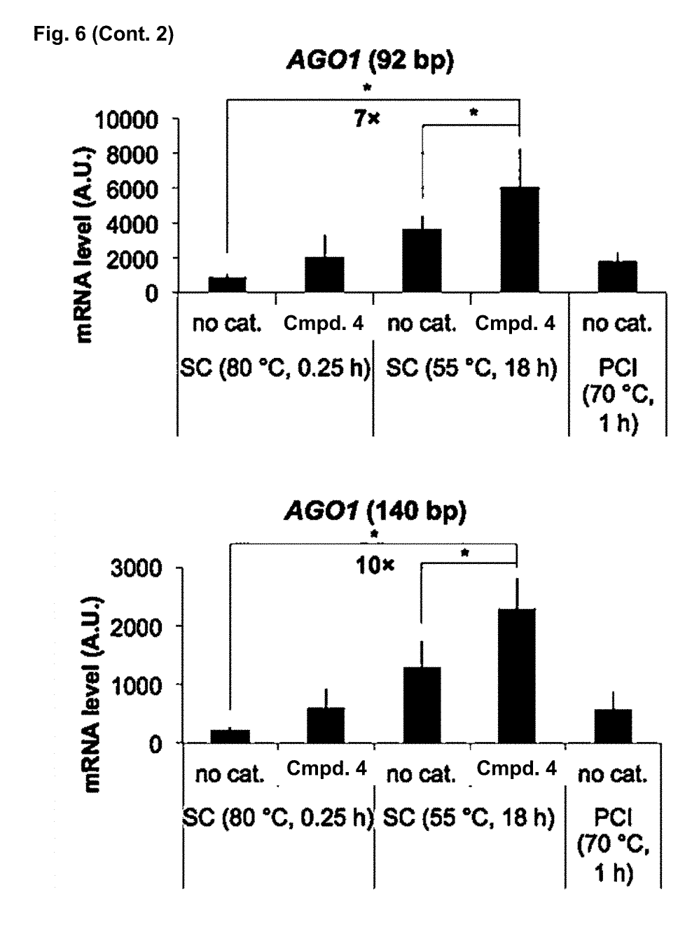

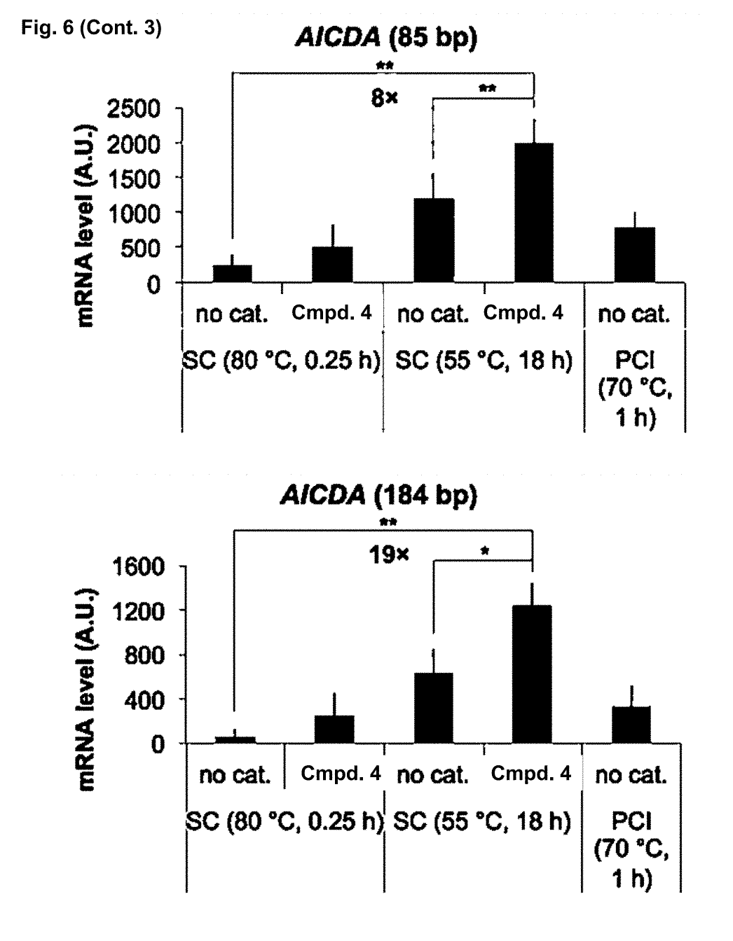

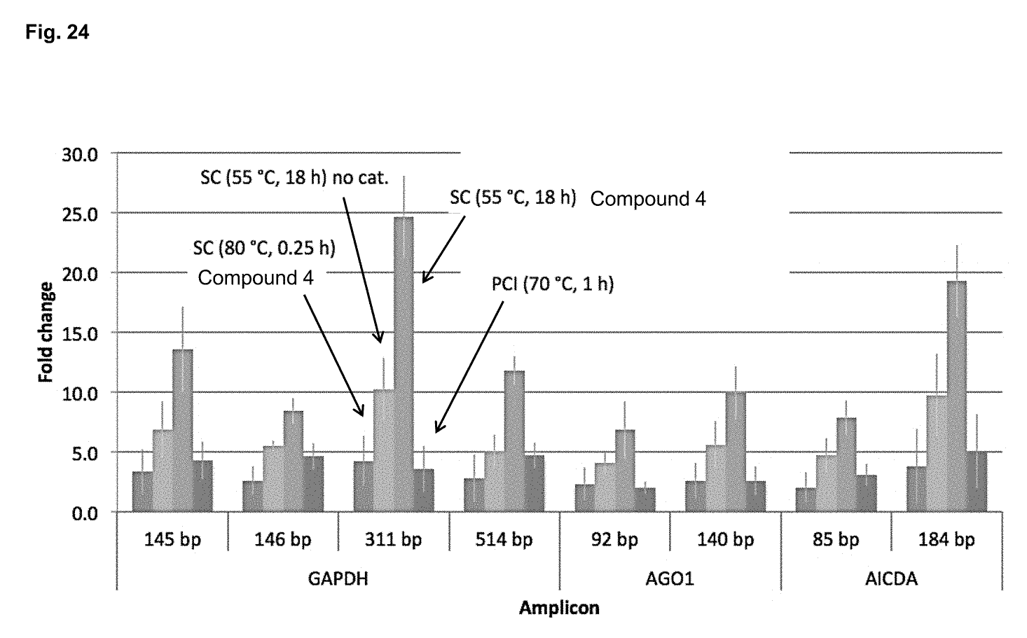

RNAs from three different genes (GAPDH, AGO1, AICDA) and with varied copy number and amplicon lengths (85-514 bp) were quantified, comparing the commercial kit protocol (which employs heating at 80° C.) to the widely used literature protocol²⁸ (70° C.), and to the optimized catalyst protocol, which uses longer incubation times at milder temperatures. Experiments revealed (FIG. 6) that for all eight amplicons tested, amplifiable RNA yields were enhanced by the catalyst protocol relative to the commercial kit and the literature procedure. The amount of amplifiable RNA was increased by a factor of 7 over the commercial method in the least efficient case (FIG. 24) and by 25-fold in the most efficient case, with an average enhancement of 13-fold. The enhancements in amplicon quantity relative to the commercial kit and the control without catalyst were statistically significant in all cases (P<0.05, 1-tailed paired samples t-test) (FIG. 6). While all amplicons benefitted from the catalytic method, longer RNA amplicons showed the greatest increase in quantities retrieved: the three longest (180-514 bp) showed a mean enhancement of 18-fold over the kit.

### Mechanistic Considerations

Taken together, our data show that organic water-soluble catalysts containing arylamines and proton donors are effective at speeding the reversal of formaldehyde-derived hemiaminal and aminal adducts from RNA and DNA bases, both for simple model systems and for cellular RNAs as well. Although many previous studies have attempted to enhance biomolecule recovery from formalin by varying buffers and heating protocols (reference 6), we are aware of no prior studies that make use of catalysts directed at the chemical mechanism of hemiaminal/aminal breakdown. The present studies suggest that Tris buffer—perhaps the most common buffer used in RNA/DNA recovery from FFPE specimens (references 11, 21)—is not an ideal choice, mechanistically speaking, because its pKₐ (8.1) is too high to be effective as a general acid proton donor at lower pH values. Indeed, while the hemiaminal adduct is eventually reversed in Tris buffer, we find that the aminal crosslink is much more stable, and we observed very little reversal in Tris buffer alone. We find that bifunctional catalysts containing both amines and proton-donating groups are considerably more active than simple buffers alone.

Our early mechanistic studies reveal that the hemiaminal reversal is not highly pH sensitive between pH 4.5-8, and suggest that the reaction is catalyzed both by general acid and nucleophilic catalysis. Bifunctional catalysts such as Compound 3 and Compound 4 show substantial ability to speed the reaction over phosphate or Tris buffers alone, and the greater activity of Compound 4 over Compound 3 may reflect a better matching of its pKa (7.3) to solution pH. On the other hand, our results show that the aminal crosslink is indeed pH sensitive, and is catalyzed by lowered pH, suggesting a specific acid catalysis mechanism. Most catalysts and buffers tested here appear to have little effect beyond a simple protonation effect; however, very low pH values are to be avoided with nucleic acids because of their likely effect in promoting depurination (reference 29). We chose pH 4.5 here as a compromise between keeping RNA stable and promoting reaction as much as possible. At this pH we find that only one catalyst, phosphanilate Compound 4, has an additional catalytic effect over buffer alone, and our studies suggest that general acid catalysis is operative. We hypothesize that this compound is unique among those tested in having a pKₐ near that of the solution pH and also being diprotic and containing an ortho-amine group, all of which may enhance complex formation with the aminal at the transition state (see hypothesized transition state in FIG. 18). Additional mechanistic studies and comparison to new catalyst analogues will be useful in testing this hypothesis in the future.

Our experiments with full RNA strands treated with formaldehyde are in accordance with our nucleotide model studies, and show that Compound 4 is the most active of all tested in reversing hemiaminal adducts and crosslinks. We find that Compound 4 allows essentially complete removal of adducts in a shorter time and at lower temperatures than Tris buffer alone, and can do so without extended heating, which we and others^{30,31} have shown causes substantial RNA degradation. When applied to formalin-fixed cellular RNAs, Compound 4 fosters the repair and recovery at mild temperatures, and enhances amplifiable RNA yields by more than an order of magnitude.

The current studies establish the utility of organocatalysts such as Compound 3 and Compound 4 in recovery of intact nucleic acids from formalin-fixed tumor samples. There are hundreds of millions of banked FFPE samples in the US alone³², with millions of new samples generated each year from tumor biopsies and cancer surgery. With an increasing clinical focus on molecular analysis of cancers in diagnosis and treatment, the development of strategies for enhanced recovery of biomolecular information from stored tissues is an important goal.

### REFERENCES

1. Sotiriou, C., Piccart, M. J. Taking gene-expression profiling to the clinic: when will molecular signatures become relevant to patient care? Nature Rev. Cancer 7, 545-553 (2007).
2. Chin, L., Gray, J. W. Translating insights from the cancer genome into clinical practice. Nature 452, 553-563 (2014).
3. Fairley, J. A., Gilmour, K., Walsh, K. Making the most of pathological specimens: molecular diagnosis in formalin-fixed, paraffin embedded tissue. Curr. Drug Targets 13, 1475-1487 (2012).
4. McGhee, J. D., von Hippel, P. H. Formaldehyde as a probe of DNA structure. I. Reaction with exocyclic amino groups of DNA bases. Biochemistry 14, 1281-1296 (1975).
5. Krafft, A. E., Duncan, B. W., Bijwaard, K. E., Taubenberger, J. K., Lichy, J. H. Optimization of the isolation and amplification of RNA from formalin-fixed, paraffin-embedded tissue: The armed forces institute of pathology experience and literature review. Mol. Diagn. 2, 217-230 (1997).
6. Okello, J. B., Zurek, J., Devault, A. M., Kuch, M., Okwi, A. L., Sewankambo, N. K., Bimenya, G. S., Poinar, D., Poinar, H. N. Comparison of methods in the recovery of nucleic acids from archival formalin-fixed paraffin-embedded autopsy tissues. Anal. Biochem. 400, 110-117 (2010).
7. Páska, C., Bögi, K., Szilák, L., Tökés, A., Szabó, E., Sziller, I., Rigó, J., Sobel, G., Szabó, I., Kaposi-Novák, P., Kiss, A., Schaff, Z. Effect of formalin, acetone, and RNAlater fixatives on tissue preservation and different size amplicons by real-time PCR from paraffin-embedded tissue. Appl. Immunohistochem. & Mol. Morphol. 13, 234-240 (2004).
8. Linton, K. M., Hey, Y., Saunders, E., Jeziorska, M., Denton, J., Wilson, C. L., Swindell, R., Dibben, S., Miller, C. J., Pepper, S. D., Radford, J. A., Freemont, A. J. Acquisition of biologically relevant gene expression data by Affymetrix microarray analysis of archival formalin-fixed paraffin-embedded tumours. Br. J. Cancer 98, 1403-1414 (2008).
9. Shi, S. R., Shi, Y., Taylor, C. R. Antigen retrieval immunohistochemistry review and future prospects in research and diagnosis over two decades. J. Histochem. Cytochem. 59, 13-32 (2011).
10. Chen, J., Byrne, G. E. Jr., Lossos, I. S. Optimization of RNA extraction from formalin-fixed, paraffin-embedded lymphoid tissues. Diagn. Mol. Pathol. 16, 61-72 (2007).
11. Gilbert, M. T., Haselkorn, T., Bunce, M., Sanchez, J. J., Lucas, S. B., Jewell, L. D., Van Marck, E., Worobey, M. The isolation of nucleic acids from fixed, paraffin-embedded tissues—which methods are useful when? PLoS One. 2, e537 (2007).
12. Lindahl, T. Instability and decay of the primary structure of DNA. Nature 362, 709-715 (1993).
13. Kubista, M., Björkman, J., Svec, D., Sjöback, R. RNA quality matters. Eur Pharm. Rev. 17, 63-67 (2012).
14. Jencks, W. P. Mechanism and catalysis of simple carbonyl group reactions. Prog. Phys. Org. Chem. 2, 63 (1964)
15. Jencks, W. P. Studies on the mechanism of oxime and semicarbazone formation. J. Am. Chem. Soc. 81, 475-481 (1959)
16. Cordes, E. H., Jencks, W. P. Nucleophilic catalysis of semicarbazone formation by anilines. J. Am. Chem. Soc. 84, 826-831 (1962).
17. Wendeler, M., Grinberg, L., Wang, X., Dawson, P. E., Baca, M. Enhanced catalysis of oxime-based bioconjugations by substituted anilines. Bioconjugate Chem. doi: 10.1021/bc400380f (2014).
18. Rashidian, M., Mahmoodi, M. M., Shah, R., Dozier, J. K., Wagner, C. R., Distefano, M. D. A highly efficient catalyst for oxime ligation and hydrazone-oxime exchange suitable for bioconjugation. Bioconjugate Chem. 24, 333 (2013)
19. Crisalli, P., Kool, E. T. Water-soluble organocatalysts for hydrazone and oxime formation. J. Org. Chem. 78, 1184-1189 (2013).
20. Crisalli, P., Kool, E. T. Importance of ortho proton donors in catalysis of hydrazone formation. Org. Lett. 15, 1646-1649 (2013).
21. Rait, V. K., Zhang, Q., Fabris, D., Mason, J. T., O′Leary, T. J. Conversions of formaldehyde-modified 2′-deoxyadenosine 5′-monophosphate in conditions modeling formalin-fixed tissue dehydration. J. Histochem. Cytochem. 54, 301-310 (2006)
22. Chaw, Y. F., Crane, L. E., Lange, P. Isolation and identification of cross-links from formaldehyde-treated nucleic acids. Biochemistry 19, 5525-5531 (1980).
23. Dirksen, A., Dirksen, S., Hackeng, T., Dawson, P. E. Nucleophilic catalysis of hydrazone formation and transimination: implications for dynamic covalent chemistry. J. Am. Chem. Soc. 128, 15602-15603 (2006).
24. Rowland, G. B., Zhang, H., Rowland, E. B., Chennamadhavuni, S., Wang, Y., Antilla, J. C. Brønsted acid-catalyzed imine amidation. J. Am. Chem. Soc. 127, 15696-15697 (2005).
25. Kerick, M., lsau, M., Timmermann, B., Sültmann, H., Herwig, R., Krobitsch, S., Schaefer, G., Verdorfer, I., Bartsch, G., Klocker, H., Lehrach, H., Schweiger, M. R. Targeted high-throughput sequencing in clinical cancer settings: formaldehyde fixed-paraffin embedded (FFPE) tumor tissues, input amount and tumor heterogeneity. BMC Med. Genomics 4, 68 (2011).
26. Montgomery, K., Zhao, S., van de Rijn, M. & Natkunam, Y. A novel method for making ‘tissue’ microarrays from small numbers of suspension cells. Appl. Immunohistochem. Mol. Morphol. 13, 80-84 (2005).
27. Karpova, M. B., Schoumans, J., Ernberg, I., Henter, J. I., Nordenskjöld, M., Fadeel, B. Raji revisited: cytogenetics of the original Burkitt's lymphoma cell line. Leukemia 19, 159-61 (2005).
28. Masuda, N., Ohnishi, T., Kawamoto, S., Monden, M. & Okubo, K. Analysis of chemical modification of RNA from formalin-fixed samples and optimization of molecular biology applications for such samples. Nucleic Acids Res. 27, 4436-4443 (1999).
29. Lindahl, T., Nyberg, B. Rate of depurination of native deoxyribonucleic acid. Biochemistry 11, 3610-3618 (1972).
30. Brisco, M. J., Morley, A. A. Quantification of RNA integrity and its use for measurement of transcript number. Nucleic Acids Res. 40, 1-9 (2012).
31. Gingrich, J., Rubio, T., Karlak, C. Effect of RNA degradation on data quality in quantitative PCR and microarray experiments. Bio-Rad. Bull. 5452A (2006).
32. Asslaber, M., Abuja, P. M., Stark, K., Eder, J., Gottweis, H., Trauner, M., Samonigg, H., Mischinger, H. J., Schippinger, W., Berghold, A., Zatloukal. K. The genome Austria tissue bank (GATiB). Pathobiology 74, 251-258 (2007).

Additional Experimental Procedures

Materials and reagents. Adenosine monophosphate (AMP), deoxyadenosine monophosphate (dAMP), lithium perchlorate, and all organic catalysts (except Compound 4) were purchased from Sigma-Aldrich Co. Methanol-free 10% formaldehyde, EM grade was purchased from Polysciences, Inc. Solvents and reagents were purchased from Fisher Scientific, Aldrich or ACROS unless otherwise noted. 1.0 M Tris-HCI (pH 7.0) buffer was purchased from Invitrogen.

Preparation of Formaldehyde Adducts of Mononucleotides.

Synthesis of N⁶-hydroxymethyl-dAMP. Synthesis was performed following a known literature procedure.¹ 2 mM solution of dAMP (in deionized water) and 10% aq. formaldehyde (methanol free) solution were mixed in equal volume to yield a mixture of 1 mM dAMP in 5% aq. formaldehyde. The reaction mixture was stirred at room temperature for three days and then quenched by freezing at −20° C., at which temperature the reaction mixture was stored. The crude mixture was precipitated from ice cold 2% LiCIO₄ in acetone and centrifuged for 15 min at 4° C. The supernatant liquid was decanted off and washed further (twice) with ice-cold acetone. The crude mixture was evaporated to dryness under vacuum to obtain a white solid. The crude mixture was purified by reverse phase HPLC (Prosphere C18, 300 Å, 10 u, 250 mm length).

HPLC purification of N⁶-hydroxymethyl-dAMP. A small portion (˜20 mg) of crude mixture (formalin-dAMP adducts) was dissolved in ˜300 μL of 0.1 M sodium phosphate buffer (pH 7.0) and purified via HPLC. A linear gradient of 0-7% acetonitrile in 0.1 M triethylammonium acetate (TEAA) buffer (pH 7.5) for 25 min with a flow rate of 3 mL/min was employed. The column was ProSphere C18-300A 10p, length 250 mm, ID 10.0 mm. HPLC data were analyzed by measuring the peak area percent covered under each peak to determine the yield of the desired product. HPLC purified fraction for N⁶-hydroxymethyl-dAMP was isolated (retention time ˜19 min) and evaporated to dryness under vacuum.

Characterization. Formation of N⁶-hydroxymethyl-dAMP was confirmed by ESI-MS, which was in agreement with the literature.¹ The high-resolution mass of N⁶-hydroxymethyl-dAMP was found to be 362.0852 ([M+H]⁺, C₁₁H₁₇N₅O₇P, Calc. 362.0860).

Synthesis of Methylene-bis-adenosine-5′-monophosphate (dimer). 0.06 M solution of AMP (in deionized water) and 0.3 M solution of 10% formaldehyde (methanol free) in 0.2 M sodium acetate buffer (pH 4.8) were mixed in equal volume. After stirring for few minutes at room temperature the turbid reaction mixture become very clear and continued stirring for 2-3 days at room temperature. The reaction mixture was quenched and stored briefly by freezing at −20° C. After thawing, the crude mixture was precipitated from ice cold 2% LiCIO₄ in acetone and centrifuged for 15 min at 4° C. The supernatant liquid was decanted off and washed further (twice) with ice-cold acetone. The crude mixture was evaporated to dryness under vacuum to obtain a white solid. The crude mixture was purified by reverse phase HPLC.

HPLC purification of Methylene-bis-adenosine-5′-monophosphate (dimer). A small portion (˜30 mg) of crude mixture was dissolved in ˜500 μL of 0.1 M sodium phosphate buffer (pH 7.0) and purified via HPLC. A discontinuous linear gradient of acetonitrile in 0.1 M TEAA buffer (pH 7.5) was used: 0-1.0% for first 3 min, 1.0-8.0% for next 22 min, 8.0-95.0% for last 3 min. An elution rate of 3 mL/min was employed. The HPLC-purified fraction for Methylene-bis-AMP (retention time ˜25-26 min) was isolated and evaporated to dryness under vacuum. The identity of the product was confirmed using MALDI-HRMS (see FIG. 7) and proton NMR (FIG. 8).

Characterization of Methylene-bis-AMP (dimer). MALDI-HRMS m/z 707.1339 ([M+H]⁺, C₂₁H₂₉N₁₀O₁₄P₂, Calc. 707.1340; ¹H NMR (D₂O): δ8.35 (s, 2H, H2), 8.20 (s, 2H, H8), 5.95-5.97 (d, 2H, J=5.6 Hz, H1′), 5.24 (s, 2H, N6-CH₂-N6), 4.61 (t, 2H, J=5.3 Hz, H2′), 4.39-4.41 (m, 2H, H3′), 4.28-4.29 (m, 2H, H4′), 4.04-4.07 (m, 4H, H5′). Isolated product (via HPLC) contains triethylammonium acetate buffer as a major impurity from HPLC buffer.

Catalysts. All the catalysts used in this study were purchased from Sigma-Aldrich except for phosphonate Compound 4, which was prepared as described below.

Preparation of catalyst stock solutions. The required quantity of each catalyst was dissolved in ˜4 mL of water or buffer (e.g. 30 mM Tris-HCI) to prepare the desired concentration of catalyst solutions (4 mM, 5 mM, 8 mM, 16 mM, and 24 mM). Catalyst solutions using catalyst alone as buffer were prepared by dissolving the required quantity of catalyst in water or buffer solution followed by carefully adjusting the pH by titrating slowly with dilute acid or base (0.1 N aq. NaOH or HCI solution), monitoring pH with a pH meter.

Representative Example:

Preparation of 4 mL (16 mM) solution of Compound 4 in water at pH 4.5. 11.9 mg of Compound 4 was dissolved in 3.96 mL of deionized water and sonicated for a few minutes. The pH of the solution mixture was then checked using a pH meter pre-calibrated (between pH 7.0 to 4.0) using known standards. Since the pH of the catalyst solution was below 3.5, the pH of the solution was adjusted to pH 4.5 by titrating ˜30 μL of aq. NaOH (0.1 N) solution (very slowly and drop-wise using micropipet). The remaining 10 μL water was added to the catalyst mixture and the pH re-checked to be exactly 4.5.

Reverse crosslinking of monoadduct and dimer monitored by HPLC. The crude mixture (equal quantity for each batch) was purified via HPLC (same method as described above). HPLC fraction for the monoadduct (N⁶-hydroxymethyl-dAMP) was collected and evaporated almost to dryness (˜10 μL) under high vacuum. Isolated product was then transferred into 1.5 mL Eppendorf tube in which 1 mL of catalyst solution (with an adjusted pH) was added. The reaction mixture was kept stirring at room temperature or at 37° C. for one hour. 100 μL of reaction mixture was collected at one-hour intervals and injected into the HPLC (same method as for the purification of monoadduct and the dimer). The progress of the reaction (reverse cross linking) was monitored at 260 nm on HPLC. The HPLC data were analyzed by measuring the peak area percent under each peak. The decrease in peak area percent of the monoadduct or the dimer was plotted against time (hour) to compare the catalytic activity for different catalysts.

Oligoribonucleotide synthesis. An RNA oligomer containing a self-complementary region (5′-AAAAACGCGCGAAAAA-3′, 5181.31 Da) was designed and synthesized using standard β-cyanoethyl phosphoramidite chemistry and 2′-O-TBDMS-protected ribonucleosides at 1 μM scale on an Applied Biosystems 394 synthesizer. Phosphoramidites were purchased from Glen Research. Deprotection and initial purification of the RNA were carried out using Glen-Pak RNA purification columns according to the manufacturer's instructions. RNA oligonucleotides were further purified using polyacrylamide gel electrophoresis. After gel extraction, dialysis, and lyophilization, the dried RNA was redissolved in 10 mM sodium phosphate buffer, pH 7.4, to a concentration of 650 μM. A self-complementary DNA test sequence (5′-GTTCTGCAGAAC-3′, 3645.44 Da) was purchased from Stanford Peptide and Nucleic Acid facility and used without further purification.

RNA/DNA formaldehyde treatment. To 1 equivalent of RNA stock solution (650 μM or 325 μM) in a 200 μL microcentrifuge tube was added 2 equivalents of methanol-free 10% formaldehyde solution with 1 M sodium chloride; the preparation scale ranged from 3 to 60 μL. The RNA-formaldehyde mixture was incubated at room temperature for 24 hours (72 hours for gel analysis of crosslinking), after which time the RNA was isolated by ethanol precipitation. The pellet was either redissolved immediately or stored at −80° C. until use (within 24 hours).

In order to test for the reversal of formaldehyde adduct formation (post-treatment), the pellet was redissolved in water (2.2 μL per 1 μL of original 650 μM RNA stock). 2 μL of the RNA were added to 8 μL of 20 mM or 10 mM buffer stock solution and incubated at 37° C. or 60° C. for 2-24h (final buffer concentrations 8 or 16 mM; final RNA concentration ˜65 μM). Reactions were terminated by freezing, and samples were stored frozen until analysis by gel electrophoresis or mass spectrometry was carried out (as soon as possible after termination of the reaction and within 20 h).

Treatment of the DNA test sequence was carried out exactly as described for RNA, except that a carrier RNA (60 mer, 30 μM) was added to aid in ethanol precipitation after formaldehyde treatment.

RNA analysis by gel electrophoresis. Prior to formaldehyde treatment, 10 pmol of RNA were 5′-labeled with [y-³²P] ATP (PerkinElmer) using 10 U of T4 polynucleotide kinase (Invitrogen) in a 20 μL reaction volume with 1X of the provided buffer. The labelling reaction was carried out for 1 hour at 37° C., then the enzyme was denatured for 10 min at 65° C. and the RNA was isolated by ethanol precipitation. The pellet was dissolved in 100 μL of water; to this solution was added 1:1 the RNA stock solution (650 μM) as needed to create a 325 μM radiolabeled RNA stock. After formaldehyde treatment and post-treatment, an equal volume of loading buffer was added to each sample, and samples were immediately loaded onto a 20% denaturing polyacrylamide gel with 7.5 M urea. The gel was run for 1.5 hours at 30 W, then exposed overnight on a storage phosphor screen and imaged using a GE Typhoon 9410 gel imager. ImageJ was used for image analysis. In order to calculate the fraction of crosslinked RNA remaining, the band corresponding to crosslinked RNA in each lane was quantified (the size of the box remained the same for each lane) and normalized relative to a formaldehyde-treated sample with no post-treatment.

RNA analysis by MALDI-MS. After formaldehyde treatment and post-treatment as described above, 1 μL of 50 μM DNA standard (5′-TCGGATCGTGATAT-3′, 4293.86 Da; prepared by Stanford Peptide and Nucleic Acid Facility) was added to each reaction. The samples were then desalted using C18 ZipTips (EMD Millipore) as previously described² and eluted directly onto a 100-well plate, on which they were cospotted with 3-HPA matrix containing ammonium citrate. MALDI-TOF mass spectrometry analysis was carried out on an ABI Voyager-DE RP mass spectrometer in linear negative ion mode with a laser intensity of 2025 a.u. and an accelerating voltage of 25000 V. The grid and guide wire were set at 92.5% and 0.15%, respectively, and the delay time was 250 nsec. Spectra were recorded from 500 to 12000 Da. All experiments were repeated five times, with 100-200 shots per spectrum and at least two spectra taken from each spot. In each case, the spectrum with the highest signal-to-background ratio was used for subsequent analysis.

The program Data Explorer was used to extract data. 5-point Gaussian smoothing was carried out, along with automatic baseline correction, calibration relative to the internal DNA standard and peak detection with a 1% intensity cutoff. Peak data was imported to Microsoft Excel for analysis. The amount of intact RNA was measured by taking the ratio of the RNA peak height at 5181.3 Da to the reference DNA peak height at 4293.86 Da. The recovery of RNA from adducts was calculated by comparing the peak height of the intact RNA (5181.3 Da) to the sum of the peak heights of intact RNA and adducts: peaks between 5181.3 Da and 5676 Da with at least 10% intensity. Finally, degradation was quantified by considering the total non-degraded RNA (intact RNA+adducts) relative to the reference DNA.

Preparation of FFPE samples. Raji lymphoma cells (from ATCC) were grown in RPMI with 10% FBS and 1% penicillin/streptomycin in T-175 flasks. RNA was isolated from approximately 20 million cells (for use as a positive control in qPCR, see below) using the Qiagen AllPrep® DNA/RNA kit, following the manufacturer's protocol. Three pellets of approximately 110 million cells were then embedded in paraffin, following a reported procedure.²

RNA Extraction Procedures from FFPE Samples.

Using Qiaqen AllPrep® DNA/RNA FFPE kit:

RNA was extracted according to the manufacturer's protocol with minor modifications. Briefly, 10 μm sections in 1.5 mL LoBind microcentrifuge tubes (Eppendorf) were individually deparaffinised with 1 mL xylenes and washed with 1 mL 100% ethanol, then air-dried for 15-30 minutes at room temperature. Each pellet was resuspended in 150 μL Buffer PKD (Qiagen), 10 μL proteinase K solution (Qiagen) was added, and the samples were incubated at 56° C. for 15 min, then incubated on ice for 3 min. Following centrifugation, the RNA-containing supernatants were aspirated and pooled. The combined supernatant was vortexed briefly and split into 150 μL aliquots, with the number of aliquots equal to the number of initial sections, in order to reduce inter-sample variability due to varying quantities of tissue in each section.

Where appropriate, 150 μL of a 40 mM solution of Compound 4 in molecular biology grade water (adjusted to pH 7.0 with 2 M NaOH) or 150 μL molecular biology grade water (negative control) was added. The solution was incubated under the specified conditions: 80° C. for 15 min (manufacturer's procedure) or 55° C. for 18 h (optimized procedure). Isolation of RNA then proceeded according to the manufacturer's instructions, with final elution of RNA in 20 μL RNase-free water.

Phenol-Chloroform-Isoamyl Alcohol Extraction:

RNA was isolated according to the procedure described in Masuda et al. with minor modifications.³ 10 μm sections in 1.5 mL LoBind microcentrifuge tubes (Eppendorf) were individually deparaffinised with 1 mL xylenes and washed with 1 mL 100% ethanol, then air-dried for 15-30 min at room temperature. Each pellet was resuspended in 150 μL Buffer PKD (Qiagen), 10 μL proteinase K solution (Qiagen) was added, and the samples were incubated at 45° C. for 1 h.

The solutions were transferred to 1.5 mL Manual Phase Lock Gel™ Heavy tube (pre-spun at 12 000×g for 30 s, 5 PRIME). 160 μL phenol-chloroform-isoamyl alcohol (25:24:1, non-buffered, Fisher Scientific) was added, and the tubes were shaken for 20 s then centrifuged at 12 000×g for 5 min at 4° C. 160 μL chloroform was added, and the tubes were shaken for 20 s then centrifuged at 12 000×g for 5 min at 4° C. The upper (aqueous) phases were transferred to 1.5 mL LoBind microcentrifuge tubes (Eppendorf), 5 μL glycogen (RNA grade, 20 mg/mL, Thermo Scientific) was added, and the tubes were mixed by repeated inversion. 415 μL (2.5 volumes) cold (−20° C.) 100% ethanol was added, and the tubes were mixed by repeated inversion, then incubated at −20° C. for a minimum of 1 h. The tubes were centrifuged at maximum speed (c. 20 000×g) for 10 min at 4° C. The supernatant was aspirated, and 500 μL 75% ethanol was added to the pellet. The tubes were vortexed, incubated at room temperature for 10 min, then centrifuged at maximum speed for 5 min at 4° C. The supernatant was aspirated, and the pellet was air-dried for 5 min at room temperature. The pellet was resuspended in 20 μL RNase-free water, pipetting up and down a few times to aid resuspension, then incubated at 55° C. for 10 min to completely redissolve the RNA. The RNA concentration was measured using the Qubit® RNA HS assay kit (Life Technologies).

DNase I (RNase-free, Life Technologies) treatment was carried out according to the manufacturer's protocol. The samples were diluted to 200 ng/μL if necessary, and 10×DNase I buffer was added to give 1×concentration in the sample. 1 μL DNase I (2 U, as defined by Ambion) was added, and the sample was incubated at 37° C. for 30 min. EDTA solution (50 mM in water) was added to give 5 mM concentration in the sample, and the sample was incubated at 75° C. for 10 min to inactivate the DNase.

To exchange buffer, the RNA was precipitated with 2.5 volumes cold (−20° C.) 100% ethanol, and incubated at −20° C. for a minimum of 1 h. After centrifugation at maximum speed (c. 20 000×g) for 10 min at 4° C., the supernatant was aspirated, and the pellet was air-dried for 5 min at room temperature. The pellet was resuspended in 20 μL 1×TBE buffer, pipetting up and down a few times to aid resuspension. The solutions were pooled and split into 20 μL aliquots, in order to reduce inter-sample variability due to varying quantities of tissue in each section. Where appropriate, the sample was heated at 70° C. for 1 h.

RNA Quantification

RNA concentration was determined using a Qubit® fluorimeter (Life Technologies) using the Qubit® RNA HS Assay Kit.

cDNA Synthesis

cDNA was synthesized using the Invitrogen SuperScript® Ill First-Strand Synthesis System for RT-PCR (Life Technologies), according to the manufacturer's instructions. 200 nM RNA was used to prepare cDNA.

qPCR

Quantitative real-time PCR was carried out on an 7900HT Fast Real-Time PCR System (Applied Biosystems) using Power SYBR° Green PCR Master Mix (Applied Biosystems) in 384-well plates. Each measurement was performed in triplicate. Total reaction volume was 10 μL. Final primer concentration was 0.2 μM. cDNA was diluted two-fold, and 1 μL diluted cDNA was used per well. A standard curve was generated by combining equal volumes of each diluted cDNA sample to be analyzed (including cDNA prepared from RNA isolated from fresh cells) and preparing a series of 5×dilutions. cDNA prepared from RNA isolated from fresh cells was used as a positive control on each plate. Analysis was carried out using SDS 2.3 (Applied Biosytems). Threshold cycle (Cₜ) values and quantities were determined automatically by the manufacturer's software. Fold change was determined according to the equation:

fold change=quantity (test condition)/quantity (Qiagen AllPrep® DNA/RNA FFPE kit)

Primers were designed using the NCBI Primer-BLAST tool, available on-line. They were checked for efficiency by analysis of Cₜ from a serial dilution of cDNA, and for specificity by analysis of the melting curve of the PCR product and subsequent 1% agarose gel electrophoresis.

TABLE 2
Primers were as follows:

| mRNA (NCBI | | | |
|---|---|---|---|
| Reference | Amplicon | Forward primer | Reverse primer |
| Sequence) | length | (5′-3′) | (5′-3′) |
| GAPDH | 145 | CAGCCGCATCTTCTTTTG | GCAACAATATCCACTTTACCAG |
| (NM_002046.5) | | C | AGTTAA |
| | | (SEQ ID NO: xx) | (SEQ ID NO: xx) |
| | 146 | TGCCCTCAACGACCACTT | TTCCTCTTGTGCTCTTGCTGG |
| | | T | (SEQ ID NO: xx) |
| | | (SEQ ID NO: xx) | |
| | 311 | GAAATCCCATCACCATCTT | GAGTCCTTCCACGATACCAAA |
| | | CCA | G |
| | | (SEQ ID NO: xx) | (SEQ ID NO: xx) |
| | 514 | CTGAACGGGAAGCTCACT | TGGTACATGACAAGGTGCGG |
| | | GG | (SEQ ID NO: xx) |
| | | (SEQ ID NO: xx) | |
| AGO1 | 92 | GATCCCGAGCAGCGAGA | TCCCATATACCCGTGCGGAG |
| (NM_012199.2) | | GTG | (SEQ ID NO: xx) |
| | | (SEQ ID NO: xx) | |
| | 140 | CCTCCGCACGGGTATATG | GGCCAGGAGCTTGATTGGTT |
| | | G | (SEQ ID NO: xx) |
| | | (SEQ ID NO: xx) | |
| AICDA | 85 | AGACACTCTGGACACCAC | CTTAGCCCAGCGGACATTT |
| (NM_020661.2) | | TAT | (SEQ ID NO: xx) |
| | | (SEQ ID NO: xx) | |
| | 184 | AGACACTCTGGACACCAC | GCAGCCGTTCTTATTGCGA |
| | | TA | (SEQ ID NO: xx) |
| | | (SEQ ID NO: xx) | |

Synthesis of Compound 4.

2-lodo-4-methylaniline:⁴

A solution of para-toluidine (11; 1.61 g, 15 mmol) and NaHCO3 (3.78 g, 45 mmol) in H2O/CH2Cl2 1:2 (90 mL) was treated with 12 (3.81 g, 15 mmol) at 0° C. under Ar and stirred at 25° C. for 22 h. The layers were separated, and the aqueous layer was extracted with CH2Cl2 (2×30 mL). The combined organic layers were washed with brine (1×40 mL) and evaporated. Flash column chromatography (SiO2; hexane/EtOAc 100:0 to 95:5) yielded 2-iodo-4-methylaniline (2.95 g, 84%) as a brown oil.

Rf=0.22 (SiO2; hexane/EtOAc 95:5, UV 254 nm); 1H NMR (400 MHz, CDCl3): d 7.47 (dd, J=1.4,

0.7 Hz, 1 H; H—C(3)), 6.95 (dd, J=8.1, 1.9 Hz, 1 H; H—C(5)), 6.68 (d, J=8.1 Hz, 1 H; H—C(6)), 4.05-2.57 (br. s, 2 H; NH2), 2.20 ppm (s, 3 H; Me).

Diethyl (2-Amino-5-methylphenyl)phosphonate:⁵

According to ref. 4: a solution of 2-iodo-4-methylaniline (3.86 g, 16.6 mmol), diethylphosphonate (2.8 mL, 21.5 mmol), K2CO3 (4.58 g, 33.1 mmol), and N,N′-dimethylethylenediamine (0.4 mL, 3.3 mmol) in anhydrous toluene (40 mL) was degassed with Ar for 20 min, treated with Cul (158 mg, 0.83 mmol), and stirred at 110° C. for 18 h under Ar. The mixture was diluted with H2O (150 mL), and the pH set to ca. 7 by addition of 1 M aq. HCI solution. The aq. layer was extracted with EtOAc (3×150 mL), and the combined organic layers were evaporated. Flash column chromatography (SiO2; hexane/EtOAc 100:0 to 0:100) yielded the expected phosphonate ester (2.30 g, 57%) as a yellow oil.

Rf=0.55 (SiO2; EtOAc, UV 254 nm); 1H NMR (400 MHz, CDCl3): d 7.28 (d, J=14.8 Hz, 1 H; H—C(6)), 7.13 (d, J=7.3 Hz, 1 H; H—C(4)), 6.63 (br. s, 1 H; H—C(3)), 4.17-4.09 (m, 2 H; CH2CH3),

4.08-3.99 (m, 2 H; CH2CH3), 2.27 (s, 3 H; Me-C(5)), 1.33 ppm (t, J=7.1 Hz, 6 H; 2×CH2CH3).

(2-Amino-5-methylphenyl)phosphonic Acid (Compound 4)

A solution of the above phosphonate ester (500 mg, 2.06 mmol) in anhydrous MeCN (15 mL) was treated with TMS-Br (2.0 mL, 14.4 mmol) at 25° C. under Ar, stirred for 16 h, evaporated, and dried at HV. The residue was suspended in H2O (15 mL), and the precipitate collected by centrifugation, washed with H2O (2×15 mL) and EtOAc (1×15 mL), and dried at HV to yield 7 (210 mg, 55%) as a beige solid. All spectral properties matched those of the same compound synthesized by our previous route:⁶

1H NMR (400 MHz, (CD3)2SO): d 7.12 (d, J=15.6 Hz, 1 H; H—C(6)), 6.95 (d, J=8.5 Hz, 1 H; H—C(4)), 6.54 (dd, J=8.0, 6.6 Hz, 1 H; H—C(3)), 2.11 ppm (s, 3 H; Me); 31P NMR (162 MHz, (CD3)2SO): d=16.39 ppm (s).

### REFERENCES

1. Rait, V. K.; Zhang, Q.; Fabris, D.; Mason, J. T.; O'Leary, T.J. J. Histochem. Cytochem. 2006, 54, 301-310.
2. Montgomery, K., Zhao, S., van de Rijn, M. & Natkunam, Y. Appl. Immunohistochem. Mol. Morphol. 2005, 13, 80-84.
3. Masuda, N., Ohnishi, T., Kawamoto, S., Monden, M. & Okubo, K. Nucleic Acids Res. 1999, 27, 4436-4443.
4. Roman, D. S.; Takahashi, Y.; Charette, A. B. Org. Lett. 2011, 13, 3242-3245.
5. Ghalib, M.; Niaz, B.; Jones, P. G.; Heinicke, J. W. Tetrahedron Lett. 2012, 53, 5012-5014.
6. Freedman, L. D.; Doak, G. O. J. Org. Chem. 1964, 29, 2450-2451.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for removing adducts and/or crosslinks from biomolecules, the method comprising:
contacting a sample comprising aldehyde fixed biomolecules with an adduct reversal agent in an amount and for a period of time sufficient to reduce the number of aldehyde fixation related adducts and/or crosslinks in the sample, wherein the adduct reversal agent is a compound comprising an aromatic ring and at least one of: an amine and a proton-donating group.

2. The method according to claim 1, wherein the adduct reversal agent is a compound comprising an amine.

3. The method according to claim 2, wherein the amine is a primary amine or a secondary amine.

4. The method according to claim 2 or claim 3, wherein the amine is an aromatic amine.

5. The method according to any of claims 1-4, wherein the adduct reversal agent is a compound comprising a proton-donating group.

6. The method according to claim 5, wherein the adduct reversal agent comprises a proton-donating group selected from: a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, and a boronic acid group.

7. The method according to claim 5 or claim 6, wherein the pKa of the proton-donating group is in a range of from 4-8.5.

8. The method according to claim 1, wherein the adduct reversal agent is an aminobenzene having an ortho phosphonate group.

9. The method according to claim 1, wherein the adduct reversal agent is a compound selected from Table 1.

10. The method according to claim 9, wherein the adduct reversal agent is compound 4.

11. The method according to any of claims 1-10, wherein said sample is contacted with two or more adduct reversal agents, each of which comprises an aromatic ring and at least one of: an amine and a proton-donating group.

12. The method according to any of claims 1-11, wherein said contacting occurs at a temperature in a range of from 15° C. to 80° C.,

13. The method according to claim 12, wherein said contacting occurs at a temperature in a range of from 15° C. to 60° C.

14. The method according to claim 13, wherein said contacting occurs at a temperature in a range of from 20° C. to 40° C.

15. The method according to claim 14, wherein said contacting occurs at room temperature.

16. The method according to any of claims 1-15, wherein said period of time is in a range of from 20 minutes to 48 hours.

17. The method according to claim 16, wherein said period of time is in a range of from 1 hour to 18 hours.

18. The method according to any of claims 1-17, wherein during said contacting, the adduct reversal agent is at concentration in a range of from 0.01 mM to 1000 mM.

19. The method according to claim 18, wherein during said contacting, the adduct reversal agent is at concentration in a range of from 0.5 mM to 100 mM.

20. The method according to claim 19, wherein during said contacting, the adduct reversal agent is at concentration in a range of from 0.5 mM to 50 mM.

21. The method according to any of claims 1-20, wherein said contacting occurs in a solution buffered to a pH that is at or near the pKa of the adduct reversal agent.

22. The method according to any of claims 1-21, wherein said contacting occurs in a solution buffered to a pH in a range of from 4 to 8.5.

23. The method according to any of claims 1-22, wherein the aldehyde fixed biomolecules are formaldehyde fixed biomolecules.

24. The method according to any of claims 1-23, wherein the biomolecules are present in an aldehyde fixed biological sample and said contacting comprises contacting the aldehyde fixed biological sample.

25. The method according to claim 24, wherein the aldehyde fixed biological sample is a formaldehyde fixed biological sample.

26. The method according to claim 25, wherein the formaldehyde fixed biological sample is a formalin fixed paraffin embedded (FFPE) biological sample.

27. The method according to claim 26, wherein the formaldehyde fixed biological sample is treated to remove paraffin prior to contact with the adduct reversal agent.

28. The method according to any of claims 24-27, wherein the biological sample is a tissue sample.

29. The method according to claim 28, wherein the tissue sample is a biopsy specimen.

30. The method according to any of claims 24-29, wherein the method comprises, after said contacting, a step of detecting a biomolecule in the contacted biological sample.

31. The method according to any of claims 1-30, wherein the method comprises, after said contacting, a step of detecting RNA, DNA, or protein in the contacted biological sample.

32. The method according to claim 31, wherein said detecting comprises: PCR, nucleic acid amplification, nucleic acid sequencing, in situ hybridization, an antibody-based protein detection method, or a combination thereof.

33. The method according to any of claims 1-32, wherein the method comprises a step of contacting the sample with a protease.

34. A composition comprising:
(a) an adduct reversal agent comprising an aromatic ring and at least one of: an amine and a proton-donating group; and
(b) a buffer.

35. The composition of claim 34, wherein the composition is lyophilized.

36. The composition of claim 35, wherein the composition is an aqueous solution.

37. The composition of any of claims 34-36, wherein the adduct reversal agent is a compound comprising an amine.

38. The composition of claim 37, wherein the amine is a primary amine or a secondary amine.

39. The composition of claim 37 or claim 38, wherein the amine is an aromatic amine.

40. The composition of any of claims 34-39, wherein the adduct reversal agent is a compound comprising a proton-donating group.

41. The composition of claim 40, wherein the adduct reversal agent comprises a proton-donating group selected from: a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, and a boronic acid group.

42. The composition of claim 40 or claim 41, wherein the pKa of the proton-donating group is in a range of from 4-8.5.

43. The composition of any of claims 34-42, wherein the adduct reversal agent is an aminobenzene having an ortho phosphonate group.

44. The composition of any of claims 34-42, wherein the adduct reversal agent is a compound selected from Table 1.

45. The composition of claim 44, wherein the adduct reversal agent is compound 4.

46. The composition of any of claims 34-45, comprising a second adduct reversal agent comprising an aromatic ring and at least one of: an amine and a proton-donating group.

47. The composition of claim 46, wherein each of said adduct reversal agents are selected from the compounds listed in Table 1.

48. The composition of any of claims 34-47, wherein the composition further comprises an aldehyde fixed biomolecule.

49. The composition of claim 48, wherein the aldehyde fixed biomolecule is one or more biomolecules selected from: a nucleic acid, an amino acid, and a protein.

50. The composition of any of claims 34-49, further comprising a protease.

51. The composition of any of claims 34-50, further comprising a chaotropic agent.

52. The composition of any of claims 34-51, wherein the composition is an aqueous solution buffered to a pH in a range of from 4 to 8.5.

53. A composition for reducing the number of adducts and/or crosslinks from biomolecules, the composition comprising:
(a) a first adduct reversal agent comprising an aromatic ring and at least one of: an amine and a proton-donating group; and
(b) a second adduct reversal agent comprising an aromatic ring and at least one of: an amine and a proton-donating group.

54. The composition of claim 53, wherein the first and second adduct reversal agents are each selected from the compounds listed in Table 1.

55. A composition comprising:
(a) an adduct reversal agent dissolved in a buffered aqueous solution, wherein the adduct reversal agent comprises an aromatic ring and at least one of: an amine and a proton-donating group; and
(b) at least one aldehyde fixed biomolecule selected from: a nucleic acid, an amino acid, and a protein.

56. A kit for reducing the number of adducts and/or crosslinks from biomolecules, the kit comprising:
(a) an adduct reversal agent comprising an aromatic ring and at least one of: an amine and a proton-donating group; and
(b) a buffer,
wherein (a) and (b) are present in the same or separate containers.

57. The kit of claim 56, wherein at least one of (a) and (b) is lyophilized.

58. The kit of claim 56 or claim 57, wherein the buffer is an aqueous solution.

59. The kit of claim 58, wherein said aqueous solution is buffered to a pH in a range of from 4 to 8.5

60. The kit of any of claims 56-59, wherein the adduct reversal agent is a compound comprising an amine.

61. The kit of claim 60, wherein the amine is a primary amine or a secondary amine.

62. The kit of claim 60 or claim 61, wherein the amine is an aromatic amine.

63. The kit of any of claims 56-62, wherein the adduct reversal agent is a compound comprising a proton-donating group.

64. The kit of claim 63, wherein the adduct reversal agent comprises a proton-donating group selected from: a carboxylic acid group, a phosphoric acid group, a phosphonic acid group, a sulfuric acid group, a nitric acid group, a phosphonamidic acid group, a phenol group, a tetrazole group, a benzimidazolidinium group, a hydroxy-benzotriazole group, a hydroxamic acid group, and a boronic acid group.

65. The kit of claim 63 or claim 64, wherein the pKa of the proton-donating group is in a range of from 4-8.5.

66. The kit of any of claims 56-65, wherein the adduct reversal agent is an aminobenzene having an ortho phosphonate group.

67. The kit of any of claims 56-65, wherein the adduct reversal agent is a compound selected from Table 1.

68. The kit of claim 67, wherein the adduct reversal agent is compound 4

69. The kit of any of claims 56-68, further comprising a second adduct reversal agent comprising an aromatic ring and at least one of: an amine and a proton-donating group.

70. The kit of any of claims 56-69, further comprising a protease.

71. The kit of any of claims 56-70, further comprising a chaotropic agent.

72. A kit for reducing the number of adducts and/or crosslinks from biomolecules, the kit comprising:
(a) a first adduct reversal agent comprising an aromatic ring and at least one of: an amine and a proton-donating group; and
(b) a second adduct reversal agent comprising an aromatic ring and at least one of: an amine and a proton-donating group,
wherein (a) and (b) are present in the same or separate containers.

73. The kit of claim 72, wherein the first and second adduct reversal agents are each selected from the compounds of Table 1.
